(19) United States
(12) Patent Application Publication
(10) Pub. No.: US 2026/0297579 A1
(43) Pub. Date: Oct. 1, 2026

(54) NOVEL INTERNAL RIBOSOME ENTRY SITE (IRES) AND INTRON SEQUENCES

(71) Applicant: Ginkgo Bioworks, Inc., Boston, MA (US)

(72) Inventors: Smita Amarnath, Wayland, MA (US); Catherine Cifuentes-Rojas, Boston, MA (US); Dermot Harnett, Boston, MA (US); Harini Krishnan, Tewksbury, MA (US); Narendra Maheshri, Nweton, MA (US); Ekaterina Nizovtseva, Cambridge, MA (US); Luis Ramirez-Tapia, Ashland, MA (US); Adam Scheidegger, Tybee Island, GA (US)

(21) Appl. No.: 19/478,103

(22) PCT Filed: Apr. 24, 2024
(86) PCT No.: PCT/US2024/026012

§ 371 Date: Oct. 23, 2025

Related U.S. Application Data
(60) Provisional application No. 63/461,785, filed on Apr. 25, 2023.

(51) Int. Cl. C12N 15/113; C12P 19/34

(52) U.S. Cl. CPC C12N 15/113; C12P 19/34; C12N 2310/532; C12N 2330/50; C12N 2830/42; C12N 2840/203

(57) ABSTRACT

In certain aspects, the present disclosure provides nucleic acid molecules containing an internal ribosomal entry site and/or two introns for production of linear and/or circularized nucleic acid molecules, as well as methods of preparation and methods of use.

### CROSS-REFERENCE TO RELATED APPLICATION

This application claims the benefit of the U.S. Provisional Application Ser. No. 63/461,785 filed Apr. 25, 2023, the entire contents of which is incorporated herein by reference.

### REFERENCE TO SEQUENCE LISTING

This application contains a Sequence Listing that has been submitted electronically as an XML file named “GBB00525.xml.” The XML file, created on Apr. 22, 2024, is 84,529 bytes in size. The material in the XML file is hereby incorporated by reference in its entirety.

### BACKGROUND

RNAs that contain an internal ribosome entry site (IRES) located 5′ to a protein coding sequence are able to initiate translation of that protein coding sequence by a cap-independent mechanism. IRES therefore can be used to initiate translation in circular RNA (circRNA) molecules, which, because of their circular nature, are not able to have a 5′ cap. However, currently available IRES sequences are typically large in size, which can negatively impact the cargo capacity of constructs in which they appear. CircRNA molecules can form through autocatalytic ribozyme action of complementary introns encoded by primary RNA transcripts. Complementary base pairing by the introns mediates circularization via a process called back-splicing.

Nucleic acid constructs can possess certain additional limitations as well, such as an inability to support high levels of protein expression over an extended period of time. Therefore, there remains a need for novel IRES sequences and construct designs for both circular and linear nucleic acid molecules to facilitate protein translation.

### SUMMARY

In some aspects, provided herein are nucleic acid molecules comprising an internal ribosomal entry site (IRES) sequence (e.g., an IRES sequence comprising any one of the nucleic acid sequences listed in Table 1). In some embodiments, the IRES sequence is operably linked to a protein coding sequence. In some embodiments, the protein coding sequence encodes a therapeutic protein or polypeptide.

In some embodiments, the nucleic acid molecule is an RNA molecule. In some embodiments, the nucleic acid molecule is a DNA molecule.

In some embodiments, the nucleic acid molecule comprises an intron sequence pair (i.e., an initial intron sequence and a corresponding intron sequence, for example, as set forth in Table 3). In some embodiments, the initial intron sequence is located within the 5′ portion of the nucleic acid molecule (a 5′ intron sequence) and the corresponding intron sequence is located within the 3′ portion of the nucleic acid molecule (a 3′ intron sequence). In some embodiments, the initial intron sequence is located within the 3′ portion of the nucleic acid molecule (a 3′ intron sequence) and the corresponding intron sequence is located within the 5′ portion of the nucleic acid molecule (a 5′ intron sequence). Therefore, as used herein, in certain embodiments, the initial intron sequence may be a 5′ intron sequence or a 3′ intron sequence. Likewise, in some embodiments, the corresponding intron sequence can be the other intron sequence in an intron sequence pair (i.e., a 5′ intron sequence when the initial intron sequence is a 3′ intron sequence or a 3′ intron sequence when the initial intron sequence is a 5′ intron sequence. In some embodiments, the initial intron sequence is a 5′ intron sequence and the corresponding intron sequence is a 3′ intron sequence. In other embodiments, the corresponding intron sequence is a 5′ intron sequence and the initial intron sequence is a 3′ intron sequence.

In some embodiments, the nucleic acid molecule comprises a 5′ intron sequence and/or a 3′ intron sequence. In some embodiments, the 5′ intron sequence is any one of the initial intron sequences listed in Table 3. In some embodiments, the 3′ intron sequence is any one of the corresponding intron sequences listed in Table 3. In other embodiments, the 5′ intron sequence is any one of the corresponding intron sequences listed in Table 3. In some embodiments, the 3′ intron sequence is any one of the initial intron sequences listed in Table 3.

In some embodiments, the 5′ intron and the 3′ intron are self-splicing elements that produce a circular nucleic acid molecule (e.g., the 5′ intron and the 3′ intron are self-splicing elements that together can mediate the splicing of the nucleic acid molecule to produce a circular nucleic acid molecule). In some embodiments, the nucleic acid molecule is a circular nucleic acid molecule.

In some embodiments, the nucleic acid molecule further comprises an optional spacer sequence situated 3′ to the IRES sequence or between the IRES sequence and the protein coding sequence. In some embodiments, the spacer is situated 5′ to or 3′ to a component of the nucleic acid molecule or between any two components (e.g., between the 5′ intron and the 3′ intron sequences).

In some embodiments, a nucleic acid molecule comprises, in order from 5′ to 3′, a 5′ intron sequence, an optional spacer, an IRES sequence, a second optional spacer, a protein coding sequence, a third optional spacer, and a 3′ intron. In some embodiments, the nucleic acid molecule comprises 0, 1, 2, 3, or more optional spacers.

In some embodiments, the nucleic acid molecule is a linear nucleic acid molecule.

In some embodiments, the nucleic acid molecule further comprises a translation regulation motif. In some embodiments, the nucleic acid molecule further comprises a reporter sequence.

In certain aspects, this disclosure provides a nucleic acid molecule comprising any one of the initial intron sequences listed in Table 3 and any one of the corresponding intron sequences listed in Table 3.

In some embodiments, the nucleic acid molecule further comprises an IRES sequence. In some embodiments, the IRES sequence comprises any one of the nucleic acid sequences listed in Table 1. In some embodiments, the IRES sequence is operably linked to a protein coding sequence. In some embodiments, the protein coding sequence encodes a therapeutic protein or polypeptide.

In some embodiments, the nucleic acid molecule further comprises a spacer sequence. In some embodiments, the nucleic acid molecule further comprises a translation regulation motif. In some embodiments, the nucleic acid molecule further comprises a reporter sequence.

In some embodiments, the nucleic acid molecule is an RNA molecule. In some embodiments, the nucleic acid molecule is a DNA molecule.

In some embodiments, the 5′ intron and the 3′ intron are self-splicing elements that produce a circular nucleic acid molecule. In some embodiments, the 5′ intron and the 3′ intron are self-splicing elements that together are capable of mediating splicing that produces a circular nucleic acid molecule from a linear nucleic acid molecule. In some embodiments, the nucleic acid molecule is a circular nucleic acid molecule.

In certain aspects, the disclosure provides a nucleic acid molecule comprising, in 5′ to 3′ order, a 5′ intron sequence, an IRES comprising any one of the nucleic acid sequences listed in Table 1, and a 3′ intron sequence. In some embodiments, the nucleic acid molecule further comprises a protein coding sequence between the IRES and the 3′ intron sequence.

In some embodiments, the 5′ intron sequence comprises any one of the initial intron sequences listed in Table 3. In some embodiments, the 3′ intron sequence comprises any one of the corresponding intron sequences listed in Table 3. In some embodiments, a nucleic acid molecule comprises, in order from 5′ to 3′, a 5′ intron, a first spacer, an IRES, a second spacer, a coding segment, a third spacer, and a 3′ intron, wherein the nucleic acid can comprise 0, 1, 2, 3, or more spacers.

In certain aspects, the disclosure provides a nucleic acid molecule comprising, from 5′ to 3′ order, a 5′ intron sequence comprising one of the initial intron sequences listed in Table 3, an IRES, and a 3′ intron sequence comprising any one of the corresponding intron sequences listed in Table 3.

In certain aspects, the disclosure provides a nucleic acid molecule comprising, from 5′ to 3′ order, a 5′ intron sequence comprising one of the corresponding intron sequences listed in Table 3, an IRES, and a 3′ intron sequence comprising any one of the initial intron sequences listed in Table 3. In some embodiments, the nucleic acid molecule further comprises a protein coding sequence between the IRES and the corresponding 3′ intron sequence.

In some embodiments, the IRES is operably linked to the protein coding sequence. In some embodiments, the protein coding sequence encodes a therapeutic protein or polypeptide. In some embodiments, the nucleic acid molecule further includes one or more spacer sequences between the 5′ intron sequence and the 3′ intron sequence, between the 5′ intron sequence and the IRES, between the IRES and the 3′ intron sequence, between the IRES and a protein coding sequence, and/or between the protein coding sequence and the 3′ intron sequence.

In some embodiments, the nucleic acid molecule further includes a reporter sequence between the 5′ intron sequence and the 3′ intron sequence. In some embodiments, the nucleic acid molecule is an RNA molecule. In some embodiments, the nucleic acid molecule is a DNA molecule. In some embodiments, the 5′ intron sequence and the 3′ intron sequence are self-splicing elements that produce a circular nucleic acid molecule (e.g., the 5′ intron and the 3′ intron are self-splicing elements that together can mediate the splicing of the nucleic acid molecule to produce a circular nucleic acid molecule). In some embodiments, the nucleic acid molecule is a circular nucleic acid molecule. In some embodiments, the nucleic acid molecule is a linear nucleic acid molecule.

In certain aspects, the disclosure provides a construct comprising any of the nucleic acid molecules disclosed herein. In certain aspects, the disclosure provides a circular nucleic acid molecule produced by any one of the nucleic acid molecules disclosed herein. In certain aspects, the disclosure provides methods of generating circular nucleic acid molecules, the method comprising expressing any of the nucleic acid molecules, constructs, or circular nucleic acid molecules disclosed herein in a cell.

In certain aspects, provided herein are methods of expressing a protein in a cell by contacting the cell with any of the nucleic acid molecules, constructs, or circular nucleic acid molecules disclosed herein.

### BRIEF DESCRIPTION OF THE DRAWINGS

FIG. 1 is a schematic showing a DNA library containing a diverse group of IRESs designed using the mining exercise described in the Exemplification.

FIG. 2 shows the size distribution of the IRES sequences in the library described in Example 1.

FIG. 3 is a schematic showing a split GFP reporter construct for circularization screening for IRES sequences in cells described in Example 1.

FIG. 4 shows GFP fluorescence measured 48 hours by flow cytometry after transfection of the GFP construct in two types of cells (HCT116 and HepG2).

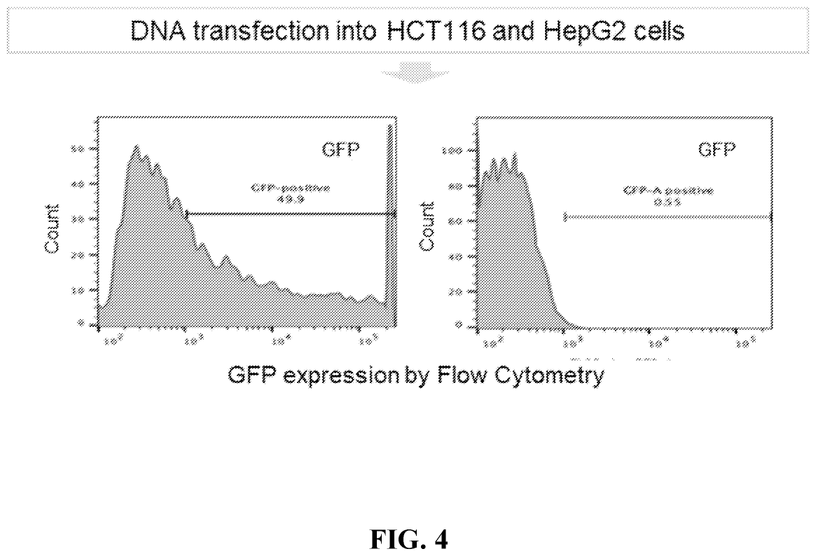

FIG. 5 is a schematic showing a construct and the strategy for the detection of circular and total RNA by qPCR.

FIG. 6 shows the level of circularization across the screened library.

FIG. 7A shows the distribution of Mean GFP Fluorescence Intensity vs IRES size in the DNA plasmid library in HCT116 and HepG2 cell lines. Samples are shown as light gray circles, the positive control is shown as a dark gray circle, and the negative control is a black circle.

FIG. 7B shows the distribution of Mean GFP Fluorescence Intensity vs IRES size in the in vitro generated RNA library in HCT116 and HepG2 cell lines. Samples are shown as light gray circles, the positive control is shown as a dark gray circle, and the negative control is a black circle.

FIG. 8 shows GFP expression from IRES sequences in the library. The top graphs correspond to Mean Fluorescence Intensity (MFI) vs IRES construct. The bottom graphs show Percent GFP positive cells vs IRES construct.

FIG. 9 is a graph showing the correlation of the GFP expression (translational output) between different cell lines (HCT116 and HepG2).

FIG. 10 shows the library composition of the IRES sequences used for assessing GFP translation of in vitro generated circular RNAs.

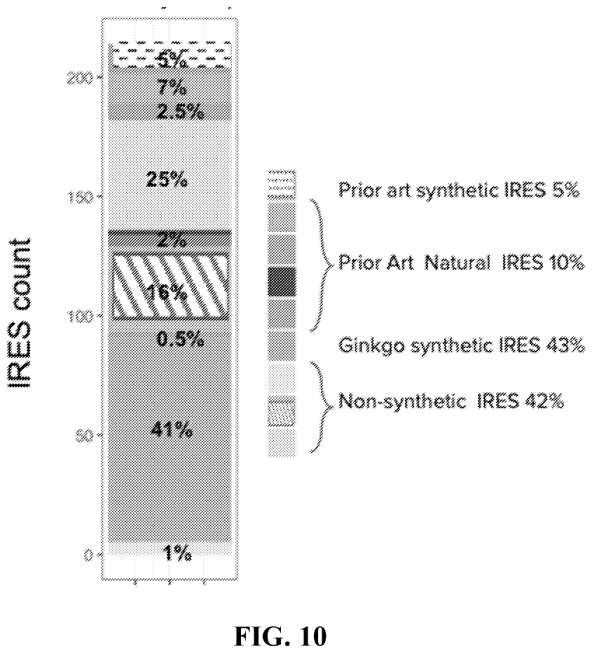

FIG. 11 is a schematic showing the GFP reporter construct for circular RNA generated in vitro for screening of IRES sequences described in Example 1.

FIG. 12 shows GFP expression from IRES sequences in the in vitro generated circular RNA library for various IRES expressed in two cell lines (HepG2 and HCT116) measured by flow cytometry at 48 h after transfection. The top graphs corresponds to Mean Fluorescence Intensity (MFI) vs IRES construct. The bottom graphs show Percent GFP positive cells vs IRES construct.

FIG. 13 is a plot showing a correlation of physical and sequence-specific characteristics used to filter intron candidates from an in silico search. The Anabaena intron is indicated as a control in the in silico strategy.

FIG. 14 is a polyacrylamide-urea electrophoresis gel showing selected intron sequences tested for in vitro splicing. Circular forms were detected (marked with an asterisk). Three introns are shown. Intron 1 was a negative hit, Intron 2 and 3 resulted in circularization in vitro. A T4 intron and a mutated T4 intron were used as positive and negative controls respectively.

FIG. 15 is a heat map showing sequence similarity of Intron hit 1, Intron hit 2, and Intron hit 3 to the T4 intron. Dark gray indicates lowest similarity, whereas light gray indicates highest sequence similarity.

FIG. 16 shows circular RNA levels measured by qPCR. Circular RNA levels were measured to be independent of IRES sequence or length.

FIG. 17 shows percent GFP expression across the modified and unmodified circular RNA library transfected in HepG2 and Hek293T cells.

### DETAILED DESCRIPTION

Internal ribosome entry site (IRES) sequences are stretches of nucleotides that recruit ribosomes independently of the canonical cap-dependent ribosomal recruitment pathway. As shown herein, Applicant developed IRES sequences that have been designed and identified as translator-drivers. Therefore, provided herein are nucleic acid molecules (e.g., synthetic or non-synthetic nucleic acid molecules) that include novel IRES sequences with translation driving capabilities and stability in production.

CircRNAs can be generated by back-splicing, wherein the 3′ terminus of a downstream exon is ligated to the 5′ terminus of an upstream exon. Circularization of RNA molecules is promoted by complementary introns that form an RNA structure to bring splice sites in proximity to facilitate ligation/circularization. Additionally, provided herein are novel self-splicing intron sequences capable of facilitating circularization of the nucleic acid molecules disclosed herein.

In some embodiments, provided herein are nucleic acid molecules comprising a novel IRES and/or a novel pair of 5′ and 3′ intron sequences. In some embodiments, the nucleic acid molecules comprising any of the novel IRES sequences disclosed herein also includes any one or more intron pairs known in the art.

As such, provided herein are nucleic acid molecules comprising an internal ribosomal entry site (IRES) sequence (e.g., an IRES sequence comprising any one of the nucleic acid sequences listed in Table 1). In some embodiments, the IRES sequence is operably linked to a protein coding sequence. Also provided herein are nucleic acid molecules including an initial intron sequence listed in Table 3 and a corresponding intron sequence listed in Table 3.

### Definitions

For convenience, certain terms employed in the specification, examples, and appended claims are collected here.

The term “amino acid” is intended to embrace molecules, whether natural or synthetic, which include both an amino functionality and an acid functionality and capable of being included in a polymer of amino acids. Example amino acids include naturally occurring amino acids; analogs, derivatives and congeners thereof; amino acid analogs having variant side chains; and stereoisomers of any of any of the foregoing. For example, “amino acid” includes proteinogenic amino acids and non-proteinogenic amino acids (NPAAs).

The term “nucleic acid molecule” refers to a polymeric form of nucleotides, either deoxyribonucleotides or ribonucleotides, or analogs thereof. The terms include single-stranded or double-stranded molecules comprising of nucleic acid bases. As such, the term includes “plasmids,” “constructs,” or “vectors.” Nucleic acid molecules may have various three-dimensional structures.

The terms “polynucleotide” and “nucleic acid” are used interchangeably. They refer to a polymeric form of nucleotides, either deoxyribonucleotides or ribonucleotides, or analogs thereof. The terms include single-stranded or double-stranded molecules comprised of nucleic acid bases. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

The terms “circular,” or “circularized,” refers to a nucleic acid molecule that forms a closed structure through covalent or non-covalent bonds. As used herein, a circular nucleic acid molecule includes a molecule that forms a covalently closed continuous loop where the 3′ and 5′ ends of a linear nucleic acid molecule have been joined. In circular nucleic acid molecules, an element or sequence that is 5′ or 3′ to another element or sequence is a determination of directionality of such elements in relation to each other. For example, an element that is 5′ to a particular element in a circular nucleic acid molecule could be any element that is closer to the particular element in the 5′ direction than that element in the 3′ direction. Conversely, an element that is 3′ to a particular element in a circular nucleic acid molecule could be any element that is closer to the particular element in the 3′ direction than that element in the 5′ direction. A person of ordinary skill in the art would understand that that “circular” may not only refer to any nucleic acid molecules with a structure like a circle, but any nucleic acid molecule with a closed structure.

The term “IRES” refers to an internal ribosomal entry site.

The term “linear,” or “linear nucleic acid molecule,” includes, but is not limited to, nucleic acid molecules having free (non-joined) 5′ and 3′ ends. In some embodiments, the linear nucleic acid has a free 5′ end and/or 3′ end. In some embodiments, the linear nucleic acid has a capped 5′ end. In some embodiments, the linear nucleic acid has a blunt end at the 5′ and/or 3′ end. In some embodiments, the linear nucleic acid has a 5′ overhang and/or a 3′ overhang. As used herein, a linear nucleic acid molecule disclosed herein is one that is not circularized or non-circular (i.e., not closed). A linear nucleic acid molecule disclosed herein may be capable of circularizing, but is not in circular form. In other embodiments, a linear nucleic acid molecule may not be capable of circularizing. For example, the linear nucleic acid molecule may lack self-splicing elements that facilitate circularization.

The term “self-splicing intron,” as used herein, refers to any intron sequence that is capable of catalyzing its own excision from parent RNA sequences without the aid of a protein molecule (e.g., a spliceosome).

Sequences are “substantially identical” or “variants thereof” if they have a specified percentage of nucleic acid residues or amino acid residues that are the same (i.e., at least 60% identity, e.g., at least 65%, 70%, 75%, 80%, 85%, 90%, 95%, 96%, 97%, 98%, or 99% identity to a reference sequence over a specified region (or the whole reference sequence when not specified)), when compared and aligned for maximum correspondence over a comparison window, or designated region as measured using any sequence comparison algorithm known in the art (GAP, BESTFIT, BLAST, Align, FASTA, and TFASTA in the Wisconsin Genetics Software Package, Genetics Computer Group (GCG), 575 Science Dr., Madison, Wis.), Karlin and Altschul Proc. Natl. Acad. Sci. (U.S.A.) 87:2264-2268 (1990) set to default settings, or by manual alignment and visual inspection (see, e.g., Ausubel et al., Current Protocols in Molecular Biology (1995-2014). Optionally, the identity exists over a region that is at least about 5, 10, 15, 20, 25, 30, 35, 40, 45, 50, 75, 100, 200, 300, 400, 500, 600, 800, 1000, or more, nucleic acids in length, or any value there between, or over the full-length of the sequence.

As used herein, the term “reporter sequence,” refers to a nucleic acid sequence that encodes for a detectable product. The expressed product itself can be detected or a metabolite or other characteristic secondarily affected by the reporter product can be detected.

The term “translational regulation motif,” as used herein, refers to a region in a sequence of a nucleic acid molecule capable of modulating the expression of a protein.

The term “therapeutic protein” or “therapeutic polypeptide” are interchangeable, and refer to a protein, a peptide, a polypeptide, or any fragment thereof that is expected to provide a positive or advantageous effect on a condition or disease state of a subject when provided to the subject. For example, a therapeutic protein or polypeptide may have curative or palliative properties and may be administered to ameliorate, relieve, alleviate, reverse, delay onset of or lessen the severity of one or more symptoms of a disease, disorder, or condition. In addition, a therapeutic protein or polypeptide will be considered therapeutic if administration of the protein is expected to delay or inhibit the progression of a disease state or condition. A therapeutic protein or polypeptide may have prophylactic properties and may be used to delay the onset of a disease. It can also include therapeutically active variants of a protein. Examples of therapeutically active proteins include, but are not limited to, cytokines, antigens for vaccination, growth factors, enzymes, hormones, inhibitors of cytokines, blood clotting factors, peptide growth, and differentiation factors. Additional, non-limiting examples of a therapeutic protein or polypeptide, as herein used, may include antigens, epitopes, or any variations thereof. A therapeutic protein or polypeptide as disclosed herein, includes and protein or polypeptide with immunogenic properties. A therapeutic protein or polypeptide as disclosed herein may be included in a vaccine. Additional non-limiting examples of therapeutic protein or polypeptides are disclosed herein.

As used herein, the term “operably linked” includes any nucleic acid sequence that is joined with a second nucleic acid sequence and is in a functional relationship with a second nucleic acid sequence. Elements need not be contiguous to be operably linked. For example, the term “operably linked”, when used in reference to a regulatory sequence and a coding sequence, means that the regulatory sequence can affect the expression of the linked coding sequence.

The term “ribozyme” refers to an RNA molecule having catalytic activity that cleaves or modifies themselves, targeted RNAs, or targeted DNAs.

### Nucleic Acid Molecules

In certain aspects, the nucleic acid molecules disclosed and described herein include an internal ribosome entry site (IRES) sequence (e.g., an IRES sequence disclosed herein). In some embodiments, the nucleic acid molecules disclosed and described herein are useful for protein synthesis using cap-independent mechanisms. In some embodiments, the nucleic acid molecules provided herein are DNA molecules, RNA molecules, or DNA-RNA hybrid molecules. In some embodiments, the nucleic acid molecules provided herein are used for circularization of RNA. In certain embodiments, provided herein are nucleic acid molecules are used for circularization of DNA. In some embodiments, the nucleic acid molecules are used for circularization of DNA-RNA hybrid molecules. In some embodiments, the nucleic acid molecules comprise an IRES sequence (e.g., an IRES sequence disclosed herein) linked (e.g., operably linked) to a protein coding sequence. In certain aspects, the disclosure provides nucleic acid molecules including a 5′ intron sequence and a 3′ intron sequence. In some embodiments, the nucleic acid molecules herein disclosed include a 5′ intron sequence listed in Table 3. In some embodiments, the nucleic acid molecules herein disclosed include a corresponding 3′ intron sequence listed in Table 3. In some embodiments, the nucleic acid molecules herein disclosed include a 5′ intron sequence listed in Table 3 and a corresponding 3′ intron sequence listed in Table 3. In certain embodiments, the disclosure provides nucleic acid molecules that include, in 5′ to 3′ order, a 5′ intron sequence, an IRES sequence listed in Table 1, and a 3′ intron sequence. In some embodiments, the 5′ intron sequence includes any one of the 5′ intron sequences listed in Table 3. In some embodiments, the 3′ intron sequence includes the corresponding 3′ intron sequence listed in Table 3. In certain aspects, the disclosure provides nucleic acid molecules including, in 5′ to 3′ order, a 5′ intron sequence having a 5′ intron sequence listed in Table 3, an IRES sequence, and a 3′ intron sequence having a corresponding 3′ intron sequence listed in Table 3. In some embodiments, the nucleic acid molecule includes a protein coding sequence between the IRES sequence and the 3′ intron sequence.

In some embodiments, the nucleic acid molecules described herein are synthetic and/or recombinant. Synthetic and/or recombinant nucleic acid molecules can be made by any known method in the art. Synthetic nucleic acid molecules can be generated as either RNA or DNA, and generated using standard techniques, such as “DNA printing” (see, for example Palluk (2018) Nature Biotechnology 36:645-650) or with dedicated devices from companies such as Kilobaser (Graz, Austria) or CureVac (Boston, MA). Synthetic nucleic acid molecules can also be ordered from companies such as Twist Biosciences (South San Francisco, CA), DNA Script (South San Francisco, CA), and Integrated DNA Technologies (Coralville, IA). While the sequences listed in the Sequence Listing are primarily listed as DNA, after converting thymine to uracil these same sequences can be used for RNA constructs.

Recombinant nucleic acid molecules, such as recombinant constructs, are generated using standard molecular biology techniques, such as those set forth in Green and Sambrook (Molecular Cloning: A Laboratory Manual, Fourth Edition, ISBN-13:978-1936113415).

In some embodiments the nucleic acid molecule is, without limitation, from 10 bp to 10 Kbp in size. In some embodiments, the nucleic acid molecule is at least 10 bp, at least 15 bp, at least 20 bp, at least 25 bp, at least 30 bp, at least 35 bp, at least 40 bp, at least 45 bp, at least 50 bp, at least 55 bp, at least 60 bp, at least 65 bp, at least 70 bp, at least 75 bp, at least 80 bp, at least 85 bp, at least 90 bp, at least 95 bp, at least 100 bp, at least 105 bp, at least 110 bp, at least 115 bp, at least 120 bp, at least 125 bp, at least 130 bp, at least 135 bp, at least 140 bp, at least 145 bp, at least 150 bp, at least 155 bp, at least 160 bp, at least 165 bp, at least 170 bp, at least 175 bp, at least 180 bp, at least 185 bp, at least 190 bp, at least 195 bp, at least 200 bp, at least 205 bp, at least 210 bp, at least 215 bp, at least 220 bp, at least 225 bp, at least 230 bp, at least 235 bp, at least 240 bp, at least 245 bp, at least 250 bp, at least 255 bp, at least 260 bp, at least 265 bp, at least 270 bp, at least 275 bp, at least 280 bp, at least 285 bp, at least 290 bp, at least 295 bp, at least 300 bp, at least 305 bp, at least 310 bp, at least 315 bp, at least 320 bp, at least 325 bp, at least 330 bp, at least 335 bp, at least 340 bp, at least 345 bp, at least 350 bp, at least 355 bp, at least 360 bp, at least 365 bp, at least 370 bp, at least 375 bp, at least 380 bp, at least 385 bp, at least 390 bp, at least 395 bp, at least 400 bp, at least 405 bp, at least 410 bp, at least 415 bp, at least 420 bp, at least 425 bp, at least 430 bp, at least 435 bp, at least 440 bp, at least 445 bp, at least 450 bp, at least 455 bp, at least 460 bp, at least 465 bp, at least 470 bp, at least 475 bp, at least 480 bp, at least 485 bp, at least 490 bp, at least 495 bp, at least 500 bp, at least 505 bp, at least 510 bp, at least 515 bp, at least 520 bp, at least 525 bp, at least 530 bp, at least 535 bp, at least 540 bp, at least 545 bp, at least 550 bp, at least 555 bp, at least 560 bp, at least 565 bp, at least 570 bp, at least 575 bp, at least 580 bp, at least 585 bp, at least 590 bp, at least 595 bp, at least 600 bp, at least 605 bp, at least 610 bp, at least 615 bp, at least 620 bp, at least 625 bp, at least 630 bp, at least 635 bp, at least 640 bp, at least 645 bp, at least 650 bp, at least 655 bp, at least 660 bp, at least 665 bp, at least 670 bp, at least 675 bp, at least 680 bp, at least 685 bp, at least 690 bp, at least 695 bp, at least 700 bp, at least 705 bp, at least 710 bp, at least 715 bp, at least 720 bp, at least 725 bp, at least 730 bp, at least 735 bp, at least 740 bp, at least 745 bp, at least 750 bp, at least 755 bp, at least 760 bp, at least 765 bp, at least 770 bp, at least 775 bp, at least 780 bp, at least 785 bp, at least 790 bp, at least 795 bp, at least 800 bp, at least 805 bp, at least 810 bp, at least 815 bp, at least 820 bp, at least 825 bp, at least 830 bp, at least 835 bp, at least 840 bp, at least 845 bp, at least 850 bp, at least 855 bp, at least 860 bp, at least 865 bp, at least 870 bp, at least 875 bp, at least 880 bp, at least 885 bp, at least 890 bp, at least 895 bp, at least 900 bp, at least 905 bp, at least 910 bp, at least 915 bp, at least 920 bp, at least 925 bp, at least 930 bp, at least 935 bp, at least 940 bp, at least 945 bp, at least 950 bp, at least 955 bp, at least 960 bp, at least 965 bp, at least 970 bp, at least 975 bp, at least 980 bp, at least 985 bp, at least 990 bp, at least 995 bp, at least 1000 bp, at least 1025 bp, at least 1050 bp, at least 1075 bp, at least 1100 bp, at least 1125 bp, at least 1150 bp, at least 1175 bp, at least 1200 bp, at least 1225 bp, at least 1250 bp, at least 1275 bp, at least 1300 bp, at least 1325 bp, at least 1350 bp, at least 1375 bp, at least 1400 bp, at least 1425 bp, at least 1450 bp, at least 1475 bp, at least 1500 bp, at least 1525 bp, at least 1550 bp, at least 1575 bp, at least 1600 bp, at least 1625 bp, at least 1650 bp, at least 1675 bp, at least 1700 bp, at least 1725 bp, at least 1750 bp, at least 1775 bp, at least 1800 bp, at least 1825 bp, at least 1850 bp, at least 1875 bp, at least 1900 bp, at least 1925 bp, at least 1950 bp, at least 1975 bp, at least 2000 bp, at least 2025 bp, at least 2050 bp, at least 2075 bp, at least 2100 bp, at least 2125 bp, at least 2150 bp, at least 2175 bp, at least 2200 bp, at least 2225 bp, at least 2250 bp, at least 2275 bp, at least 2300 bp, at least 2325 bp, at least 2350 bp, at least 2375 bp, at least 2400 bp, at least 2425 bp, at least 2450 bp, at least 2475 bp, at least 2500 bp, at least 2525 bp, at least 2550 bp, at least 2575 bp, at least 2600 bp, at least 2625 bp, at least 2650 bp, at least 2675 bp, at least 2700 bp, at least 2725 bp, at least 2750 bp, at least 2775 bp, at least 2800 bp, at least 2825 bp, at least 2850 bp, at least 2875 bp, at least 2900 bp, at least 2925 bp, at least 2950 bp, at least 2975 bp, at least 3000 bp, at least 3025 bp, at least 3050 bp, at least 3075 bp, at least 3100 bp, at least 3125 bp, at least 3150 bp, at least 3175 bp, at least 3200 bp, at least 3225 bp, at least 3250 bp, at least 3275 bp, at least 3300 bp, at least 3325 bp, at least 3350 bp, at least 3375 bp, at least 3400 bp, at least 3425 bp, at least 3450 bp, at least 3475 bp, at least 3500 bp, at least 3525 bp, at least 3550 bp, at least 3575 bp, at least 3600 bp, at least 3625 bp, at least 3650 bp, at least 3675 bp, at least 3700 bp, at least 3725 bp, at least 3750 bp, at least 3775 bp, at least 3800 bp, at least 3825 bp, at least 3850 bp, at least 3875 bp, at least 3900 bp, at least 3925 bp, at least 3950 bp, at least 3975 bp, at least 4000 bp, at least 4025 bp, at least 4050 bp, at least 4075 bp, at least 4100 bp, at least 4125 bp, at least 4150 bp, at least 4175 bp, at least 4200 bp, at least 4225 bp, at least 4250 bp, at least 4275 bp, at least 4300 bp, at least 4325 bp, at least 4350 bp, at least 4375 bp, at least 4400 bp, at least 4425 bp, at least 4450 bp, at least 4475 bp, at least 4500 bp, at least 4525 bp, at least 4550 bp, at least 4575 bp, at least 4600 bp, at least 4625 bp, at least 4650 bp, at least 4675 bp, at least 4700 bp, at least 4725 bp, at least 4750 bp, at least 4775 bp, at least 4800 bp, at least 4825 bp, at least 4850 bp, at least 4875 bp, at least 4900 bp, at least 4925 bp, at least 4950 bp, at least 4975 bp, at least 5000 bp, at least 5025 bp, at least 5050 bp, at least 5075 bp, at least 5100 bp, at least 5125 bp, at least 5150 bp, at least 5175 bp, at least 5200 bp, at least 5225 bp, at least 5250 bp, at least 5275 bp, at least 5300 bp, at least 5325 bp, at least 5350 bp, at least 5375 bp, at least 5400 bp, at least 5425 bp, at least 5450 bp, at least 5475 bp, at least 5500 bp, at least 5525 bp, at least 5550 bp, at least 5575 bp, at least 5600 bp, at least 5625 bp, at least 5650 bp, at least 5675 bp, at least 5700 bp, at least 5725 bp, at least 5750 bp, at least 5775 bp, at least 5800 bp, at least 5825 bp, at least 5850 bp, at least 5875 bp, at least 5900 bp, at least 5925 bp, at least 5950 bp, at least 5975 bp, at least 6000 bp, at least 6025 bp, at least 6050 bp, at least 6075 bp, at least 6100 bp, at least 6125 bp, at least 6150 bp, at least 6175 bp, at least 6200 bp, at least 6225 bp, at least 6250 bp, at least 6275 bp, at least 6300 bp, at least 6325 bp, at least 6350 bp, at least 6375 bp, at least 6400 bp, at least 6425 bp, at least 6450 bp, at least 6475 bp, at least 6500 bp, at least 6525 bp, at least 6550 bp, at least 6575 bp, at least 6600 bp, at least 6625 bp, at least 6650 bp, at least 6675 bp, at least 6700 bp, at least 6725 bp, at least 6750 bp, at least 6775 bp, at least 6800 bp, at least 6825 bp, at least 6850 bp, at least 6875 bp, at least 6900 bp, at least 6925 bp, at least 6950 bp, at least 6975 bp, at least 7000 bp, at least 7025 bp, at least 7050 bp, at least 7075 bp, at least 7100 bp, at least 7125 bp, at least 7150 bp, at least 7175 bp, at least 7200 bp, at least 7225 bp, at least 7250 bp, at least 7275 bp, at least 7300 bp, at least 7325 bp, at least 7350 bp, at least 7375 bp, at least 7400 bp, at least 7425 bp, at least 7450 bp, at least 7475 bp, at least 7500 bp, at least 7525 bp, at least 7550 bp, at least 7575 bp, at least 7600 bp, at least 7625 bp, at least 7650 bp, at least 7675 bp, at least 7700 bp, at least 7725 bp, at least 7750 bp, at least 7775 bp, at least 7800 bp, at least 7825 bp, at least 7850 bp, at least 7875 bp, at least 7900 bp, at least 7925 bp, at least 7950 bp, at least 7975 bp, at least 8000 bp, at least 8025 bp, at least 8050 bp, at least 8075 bp, at least 8100 bp, at least 8125 bp, at least 8150 bp, at least 8175 bp, at least 8200 bp, at least 8225 bp, at least 8250 bp, at least 8275 bp, at least 8300 bp, at least 8325 bp, at least 8350 bp, at least 8375 bp, at least 8400 bp, at least 8425 bp, at least 8450 bp, at least 8475 bp, at least 8500 bp, at least 8525 bp, at least 8550 bp, at least 8575 bp, at least 8600 bp, at least 8625 bp, at least 8650 bp, at least 8675 bp, at least 8700 bp, at least 8725 bp, at least 8750 bp, at least 8775 bp, at least 8800 bp, at least 8825 bp, at least 8850 bp, at least 8875 bp, at least 8900 bp, at least 8925 bp, at least 8950 bp, at least 8975 bp, at least 9000 bp, at least 9025 bp, at least 9050 bp, at least 9075 bp, at least 9100 bp, at least 9125 bp, at least 9150 bp, at least 9175 bp, at least 9200 bp, at least 9225 bp, at least 9250 bp, at least 9275 bp, at least 9300 bp, at least 9325 bp, at least 9350 bp, at least 9375 bp, at least 9400 bp, at least 9425 bp, at least 9450 bp, at least 9475 bp, at least 9500 bp, at least 9525 bp, at least 9550 bp, at least 9575 bp, at least 9600 bp, at least 9625 bp, at least 9650 bp, at least 9675 bp, at least 9700 bp, at least 9725 bp, at least 9750 bp, at least 9775 bp, at least 9800 bp, at least 9825 bp, at least 9850 bp, at least 9875 bp, at least 9900 bp, at least 9925 bp, at least 9950 bp, at least 9975 bp, at least 10000 bp. For example, in some embodiments, the nucleic acid molecule is between 200 bp and 10 kbp, between 300 bp and 10 kbp between 400 bp and 10 kbp, between 500 bp and 10 kbp, 600 bp and 10 kbp, between 700 bp and 10 kbp between 800 bp and 10 kbp, between 900 bp and 10 kbp, 1 kbp and 10 kbp, between 2 kbp and 10 kbp between 3 kbp and 10 kbp, 4 kbp and 10 kbp, between 5 kbp and 10 kbp between 6 kbp and 10 kbp, 7 kbp and 10 kbp, between 8 kbp and 10 kbp or between 9 kbp and 10 kbp.

In some embodiments, the nucleic acid molecule is no more than 300 bp, 305 bp, 310 bp, 315 bp, 320 bp, 325 bp, 330 bp, 335 bp, 340 bp, 345 bp, 350 bp, 355 bp, 360 bp, 365 bp, 370 bp, 375 bp, 380 bp, 385 bp, 390 bp, 395 bp, 400 bp, 405 bp, 410 bp, 415 bp, 420 bp, 425 bp, 430 bp, 435 bp, 440 bp, 445 bp, 450 bp, 455 bp, 460 bp, 465 bp, 470 bp, 475 bp, 480 bp, 485 bp, 490 bp, 495 bp, 500 bp, 505 bp, 510 bp, 515 bp, 520 bp, 525 bp, 530 bp, 535 bp, 540 bp, 545 bp, 550 bp, 555 bp, 560 bp, 565 bp, 570 bp, 575 bp, 580 bp, 585 bp, 590 bp, 595 bp, 600 bp, 605 bp, 610 bp, 615 bp, 620 bp, 625 bp, 630 bp, 635 bp, 640 bp, 645 bp, 650 bp, 655 bp, 660 bp, 665 bp, 670 bp, 675 bp, 680 bp, 685 bp, 690 bp, 695 bp, 700 bp, 705 bp, 710 bp, 715 bp, 720 bp, 725 bp, 730 bp, 735 bp, 740 bp, 745 bp, 750 bp, 755 bp, 760 bp, 765 bp, 770 bp, 775 bp, 780 bp, 785 bp, 790 bp, 795 bp, 800 bp, 805 bp, 810 bp, 815 bp, 820 bp, 825 bp, 830 bp, 835 bp, 840 bp, 845 bp, 850 bp, 855 bp, 860 bp, 865 bp, 870 bp, 875 bp, 880 bp, 885 bp, 890 bp, 895 bp, 900 bp, 905 bp, 910 bp, 915 bp, 920 bp, 925 bp, 930 bp, 935 bp, 940 bp, 945 bp, 950 bp, 955 bp, 960 bp, 965 bp, 970 bp, 975 bp, 980 bp, 985 bp, 990 bp, 995 bp, 1000 bp, 1025 bp, 1050 bp, 1075 bp, 1100 bp, 1125 bp, 1150 bp, 1175 bp, 1200 bp, 1225 bp, 1250 bp, 1275 bp, 1300 bp, 1325 bp, 1350 bp, 1375 bp, 1400 bp, 1425 bp, 1450 bp, 1475 bp, 1500 bp, 1525 bp, 1550 bp, 1575 bp, 1600 bp, 1625 bp, 1650 bp, 1675 bp, 1700 bp, 1725 bp, 1750 bp, 1775 bp, 1800 bp, 1825 bp, 1850 bp, 1875 bp, 1900 bp, 1925 bp, 1950 bp, 1975 bp, 2000 bp, 2025 bp, 2050 bp, 2075 bp, 2100 bp, 2125 bp, 2150 bp, 2175 bp, 2200 bp, 2225 bp, 2250 bp, 2275 bp, 2300 bp, 2325 bp, 2350 bp, 2375 bp, 2400 bp, 2425 bp, 2450 bp, 2475 bp, 2500 bp, 2525 bp, 2550 bp, 2575 bp, 2600 bp, 2625 bp, 2650 bp, 2675 bp, 2700 bp, 2725 bp, 2750 bp, 2775 bp, 2800 bp, 2825 bp, 2850 bp, 2875 bp, 2900 bp, 2925 bp, 2950 bp, 2975 bp, 3000 bp, 3025 bp, 3050 bp, 3075 bp, 3100 bp, 3125 bp, 3150 bp, 3175 bp, 3200 bp, 3225 bp, 3250 bp, 3275 bp, 3300 bp, 3325 bp, 3350 bp, 3375 bp, 3400 bp, 3425 bp, 3450 bp, 3475 bp, 3500 bp, 3525 bp, 3550 bp, 3575 bp, 3600 bp, 3625 bp, 3650 bp, 3675 bp, 3700 bp, 3725 bp, 3750 bp, 3775 bp, 3800 bp, 3825 bp, 3850 bp, 3875 bp, 3900 bp, 3925 bp, 3950 bp, 3975 bp, 4000 bp, 4025 bp, 4050 bp, 4075 bp, 4100 bp, 4125 bp, 4150 bp, 4175 bp, 4200 bp, 4225 bp, 4250 bp, 4275 bp, 4300 bp, 4325 bp, 4350 bp, 4375 bp, 4400 bp, 4425 bp, 4450 bp, 4475 bp, 4500 bp, 4525 bp, 4550 bp, 4575 bp, 4600 bp, 4625 bp, 4650 bp, 4675 bp, 4700 bp, 4725 bp, 4750 bp, 4775 bp, 4800 bp, 4825 bp, 4850 bp, 4875 bp, 4900 bp, 4925 bp, 4950 bp, 4975 bp, 5000 bp, 5025 bp, 5050 bp, 5075 bp, 5100 bp, 5125 bp, 5150 bp, 5175 bp, 5200 bp, 5225 bp, 5250 bp, 5275 bp, 5300 bp, 5325 bp, 5350 bp, 5375 bp, 5400 bp, 5425 bp, 5450 bp, 5475 bp, 5500 bp, 5525 bp, 5550 bp, 5575 bp, 5600 bp, 5625 bp, 5650 bp, 5675 bp, 5700 bp, 5725 bp, 5750 bp, 5775 bp, 5800 bp, 5825 bp, 5850 bp, 5875 bp, 5900 bp, 5925 bp, 5950 bp, 5975 bp, 6000 bp, 6025 bp, 6050 bp, 6075 bp, 6100 bp, 6125 bp, 6150 bp, 6175 bp, 6200 bp, 6225 bp, 6250 bp, 6275 bp, 6300 bp, 6325 bp, 6350 bp, 6375 bp, 6400 bp, 6425 bp, 6450 bp, 6475 bp, 6500 bp, 6525 bp, 6550 bp, 6575 bp, 6600 bp, 6625 bp, 6650 bp, 6675 bp, 6700 bp, 6725 bp, 6750 bp, 6775 bp, 6800 bp, 6825 bp, 6850 bp, 6875 bp, 6900 bp, 6925 bp, 6950 bp, 6975 bp, 7000 bp, 7025 bp, 7050 bp, 7075 bp, 7100 bp, 7125 bp, 7150 bp, 7175 bp, 7200 bp, 7225 bp, 7250 bp, 7275 bp, 7300 bp, 7325 bp, 7350 bp, 7375 bp, 7400 bp, 7425 bp, 7450 bp, 7475 bp, 7500 bp, 7525 bp, 7550 bp, 7575 bp, 7600 bp, 7625 bp, 7650 bp, 7675 bp, 7700 bp, 7725 bp, 7750 bp, 7775 bp, 7800 bp, 7825 bp, 7850 bp, 7875 bp, 7900 bp, 7925 bp, 7950 bp, 7975 bp, 8000 bp, 8025 bp, 8050 bp, 8075 bp, 8100 bp, 8125 bp, 8150 bp, 8175 bp, 8200 bp, 8225 bp, 8250 bp, 8275 bp, 8300 bp, 8325 bp, 8350 bp, 8375 bp, 8400 bp, 8425 bp, 8450 bp, 8475 bp, 8500 bp, 8525 bp, 8550 bp, 8575 bp, 8600 bp, 8625 bp, 8650 bp, 8675 bp, 8700 bp, 8725 bp, 8750 bp, 8775 bp, 8800 bp, 8825 bp, 8850 bp, 8875 bp, 8900 bp, 8925 bp, 8950 bp, 8975 bp, 9000 bp, 9025 bp, 9050 bp, 9075 bp, 9100 bp, 9125 bp, 9150 bp, 9175 bp, 9200 bp, 9225 bp, 9250 bp, 9275 bp, 9300 bp, 9325 bp, 9350 bp, 9375 bp, 9400 bp, 9425 bp, 9450 bp, 9475 bp, 9500 bp, 9525 bp, 9550 bp, 9575 bp, 9600 bp, 9625 bp, 9650 bp, 9675 bp, 9700 bp, 9725 bp, 9750 bp, 9775 bp, 9800 bp, 9825 bp, 9850 bp, 9875 bp, 9900 bp, 9925 bp, 9950 bp, 9975 bp, or 10000 bp in length.

In some embodiments, a binding site is present in the nucleic acid molecule; for example, the binding site can bind a primer for reverse transcription, an RNA polymerase, a transcription factor, and/or combinations thereof.

Nucleic acid molecules herein provided can be assessed using in vitro transcription (IVT) according to standard protocols. For example, once the constructs are assembled, PCR can be conducted with an upstream primer containing an RNA polymerase promoter to amplify the nucleic acid molecule and provide an IVT template. IVT is then performed using an appropriate RNA polymerase. Many suitable reverse transcriptases/RNA polymerases are available commercially, such as T7, T3, and SP6, to name but a few. Typically, the IVT reaction is conducted for at least 1 hour or can be allowed to reach equilibrium. The resulting RNA fragments can be assessed on denaturing agarose or acrylamide gels, as well as on non-denaturing gels, with aptamers that bind a fluorophore, or via qRTPCR.

### Circular Nucleic Acid Molecules

In some embodiments, the nucleic acid molecule is a circular nucleic acid molecule. In some embodiments, the circular nucleic acid molecule comprises an IRES sequence (e.g., an IRES sequence listed in Table 1). In some embodiments, the circular nucleic acid molecule is a circular RNA molecule. In some embodiments, the circular nucleic acid molecule is a circular DNA molecule. In some embodiments, the circular nucleic acid molecule is a circular hybrid molecule comprising DNA and RNA nucleotides.

In some embodiments, the nucleic acid molecules disclosed herein include introns that facilitate circularization of the nucleic acid molecules. In some embodiments, the 5′ intron and the 3′ intron are self-splicing elements that produce the circular nucleic acid molecule (e.g., the initial and corresponding intron sequences disclosed in Table 3). In some embodiments, the 5′ intron and the 3′ intron together produce the circular nucleic acid molecule. In some embodiments, the 5′ intron and the 3′ intron are capable of mediating splicing that produces a circular nucleic acid molecule from a linear or non-closed nucleic acid molecule. A circular nucleic acid molecule is less susceptible to degradation by exonucleases and hence has increased stability. The increased stability of the circular nucleic acid molecule makes it useful as a cell transforming reagent to produce polypeptides, and allows easy storage for extended periods of time. The stability of the circular nucleic acid molecule treated with exonuclease can be tested using methods standard in the art to determine whether nucleic acid degradation has occurred (e.g., by gel electrophoresis).

In some embodiments, the circular nucleic acid molecule lacks an enzymatic cleavage site. In some embodiments, the circular nucleic acid molecule has a half-life at least about 5%, at least about 10%, at least about 20%, at least about 30%, at least about 40%, at least about 50%, at least about 60%, at least about 70%, at least about 80%, at least about 90%, at least about 100%, at least about 120%, at least about 140%, at least about 150%, at least about 160%, at least about 180%, at least about 200%, at least about 300%, at least about 400%, at least about 500%, at least about 600%, at least about 700% at least about 800%, at least about 900%, at least about 1000% or at least about 10000%, longer than a reference, e.g., a linear counterpart having the same nucleotide sequence but that is not circularized. Moreover, the circular nucleic acid molecule is less susceptible to dephosphorylation when the circular nucleic acid molecule is incubated with a phosphatase, such as calf intestine phosphatase. Therefore, in some embodiments, the circular nucleic acid molecule is incubated with a phosphatase.

In some embodiments, the circular nucleic acid molecule is about 500, 1000, 2000, 3,000, 4000, 5000, or 6000 nucleotides in size. In some embodiments, the circular nucleic acid molecule is at least 25 nucleotides, at least 50 nucleotides, at least 75 nucleotides, at least 100 nucleotides, at least 125 nucleotides, at least 150 nucleotides, at least 175 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides, at least 400 nucleotides, at least 425 nucleotides, at least 450 nucleotides, at least 475 nucleotides, at least 500 nucleotides, at least 525 nucleotides, at least 550 nucleotides, at least 575 nucleotides, at least 600 nucleotides, at least 625 nucleotides, at least 650 nucleotides, at least 675 nucleotides, at least 700 nucleotides, at least 725 nucleotides, at least 750 nucleotides, at least 775 nucleotides, at least 800 nucleotides, at least 825 nucleotides, at least 850 nucleotides, at least 875 nucleotides, at least 900 nucleotides, at least 925 nucleotides, at least 950 nucleotides, at least 975 nucleotides, at least 1000 nucleotides, at least 1025 nucleotides, at least 1050 nucleotides, at least 1075 nucleotides, at least 1100 nucleotides, at least 1125 nucleotides, at least 1150 nucleotides, at least 1175 nucleotides, at least 1200 nucleotides, at least 1225 nucleotides, at least 1250 nucleotides, at least 1275 nucleotides, at least 1300 nucleotides, at least 1325 nucleotides, at least 1350 nucleotides, at least 1375 nucleotides, at least 1400 nucleotides, at least 1425 nucleotides, at least 1450 nucleotides, at least 1475 nucleotides, at least 1500 nucleotides, at least 1525 nucleotides, at least 1550 nucleotides, at least 1575 nucleotides, at least 1600 nucleotides, at least 1625 nucleotides, at least 1650 nucleotides, at least 1675 nucleotides, at least 1700 nucleotides, at least 1725 nucleotides, at least 1750 nucleotides, at least 1775 nucleotides, at least 1800 nucleotides, at least 1825 nucleotides, at least 1850 nucleotides, at least 1875 nucleotides, at least 1900 nucleotides, at least 1925 nucleotides, at least 1950 nucleotides, at least 1975 nucleotides, at least 2000 nucleotides, at least 2025 nucleotides, at least 2050 nucleotides, at least 2075 nucleotides, at least 2100 nucleotides, at least 2125 nucleotides, at least 2150 nucleotides, at least 2175 nucleotides, at least 2200 nucleotides, at least 2225 nucleotides, at least 2250 nucleotides, at least 2275 nucleotides, at least 2300 nucleotides, at least 2325 nucleotides, at least 2350 nucleotides, at least 2375 nucleotides, at least 2400 nucleotides, at least 2425 nucleotides, at least 2450 nucleotides, at least 2475 nucleotides, at least 2500 nucleotides, at least 2525 nucleotides, at least 2550 nucleotides, at least 2575 nucleotides, at least 2600 nucleotides, at least 2625 nucleotides, at least 2650 nucleotides, at least 2675 nucleotides, at least 2700 nucleotides, at least 2725 nucleotides, at least 2750 nucleotides, at least 2775 nucleotides, at least 2800 nucleotides, at least 2825 nucleotides, at least 2850 nucleotides, at least 2875 nucleotides, at least 2900 nucleotides, at least 2925 nucleotides, at least 2950 nucleotides, at least 2975 nucleotides, at least 3000 nucleotides, at least 3025 nucleotides, at least 3050 nucleotides, at least 3075 nucleotides, at least 3100 nucleotides, at least 3125 nucleotides, at least 3150 nucleotides, at least 3175 nucleotides, at least 3200 nucleotides, at least 3225 nucleotides, at least 3250 nucleotides, at least 3275 nucleotides, at least 3300 nucleotides, at least 3325 nucleotides, at least 3350 nucleotides, at least 3375 nucleotides, at least 3400 nucleotides, at least 3425 nucleotides, at least 3450 nucleotides, at least 3475 nucleotides, at least 3500 nucleotides, at least 3525 nucleotides, at least 3550 nucleotides, at least 3575 nucleotides, at least 3600 nucleotides, at least 3625 nucleotides, at least 3650 nucleotides, at least 3675 nucleotides, at least 3700 nucleotides, at least 3725 nucleotides, at least 3750 nucleotides, at least 3775 nucleotides, at least 3800 nucleotides, at least 3825 nucleotides, at least 3850 nucleotides, at least 3875 nucleotides, at least 3900 nucleotides, at least 3925 nucleotides, at least 3950 nucleotides, at least 3975 nucleotides, at least 4000 nucleotides, at least 4025 nucleotides, at least 4050 nucleotides, at least 4075 nucleotides, at least 4100 nucleotides, at least 4125 nucleotides, at least 4150 nucleotides, at least 4175 nucleotides, at least 4200 nucleotides, at least 4225 nucleotides, at least 4250 nucleotides, at least 4275 nucleotides, at least 4300 nucleotides, at least 4325 nucleotides, at least 4350 nucleotides, at least 4375 nucleotides, at least 4400 nucleotides, at least 4425 nucleotides, at least 4450 nucleotides, at least 4475 nucleotides, at least 4500 nucleotides, at least 4525 nucleotides, at least 4550 nucleotides, at least 4575 nucleotides, at least 4600 nucleotides, at least 4625 nucleotides, at least 4650 nucleotides, at least 4675 nucleotides, at least 4700 nucleotides, at least 4725 nucleotides, at least 4750 nucleotides, at least 4775 nucleotides, at least 4800 nucleotides, at least 4825 nucleotides, at least 4850 nucleotides, at least 4875 nucleotides, at least 4900 nucleotides, at least 4925 nucleotides, at least 4950 nucleotides, at least 4975 nucleotides, at least 5000 nucleotides, at least 5025 nucleotides, at least 5050 nucleotides, at least 5075 nucleotides, at least 5100 nucleotides, at least 5125 nucleotides, at least 5150 nucleotides, at least 5175 nucleotides, at least 5200 nucleotides, at least 5225 nucleotides, at least 5250 nucleotides, at least 5275 nucleotides, at least 5300 nucleotides, at least 5325 nucleotides, at least 5350 nucleotides, at least 5375 nucleotides, at least 5400 nucleotides, at least 5425 nucleotides, at least 5450 nucleotides, at least 5475 nucleotides, at least 5500 nucleotides, at least 5525 nucleotides, at least 5550 nucleotides, at least 5575 nucleotides, at least 5600 nucleotides, at least 5625 nucleotides, at least 5650 nucleotides, at least 5675 nucleotides, at least 5700 nucleotides, at least 5725 nucleotides, at least 5750 nucleotides, at least 5775 nucleotides, at least 5800 nucleotides, at least 5825 nucleotides, at least 5850 nucleotides, at least 5875 nucleotides, at least 5900 nucleotides, at least 5925 nucleotides, at least 5950 nucleotides, at least 5975 nucleotides, or at least 6000 nucleotides.

In some embodiments, the circular nucleic acid molecule is no more than 500 bp, 505 bp, 510 bp, 515 bp, 520 bp, 525 bp, 530 bp, 535 bp, 540 bp, 545 bp, 550 bp, 555 bp, 560 bp, 565 bp, 570 bp, 575 bp, 580 bp, 585 bp, 590 bp, 595 bp, 600 bp, 605 bp, 610 bp, 615 bp, 620 bp, 625 bp, 630 bp, 635 bp, 640 bp, 645 bp, 650 bp, 655 bp, 660 bp, 665 bp, 670 bp, 675 bp, 680 bp, 685 bp, 690 bp, 695 bp, 700 bp, 705 bp, 710 bp, 715 bp, 720 bp, 725 bp, 730 bp, 735 bp, 740 bp, 745 bp, 750 bp, 755 bp, 760 bp, 765 bp, 770 bp, 775 bp, 780 bp, 785 bp, 790 bp, 795 bp, 800 bp, 805 bp, 810 bp, 815 bp, 820 bp, 825 bp, 830 bp, 835 bp, 840 bp, 845 bp, 850 bp, 855 bp, 860 bp, 865 bp, 870 bp, 875 bp, 880 bp, 885 bp, 890 bp, 895 bp, 900 bp, 905 bp, 910 bp, 915 bp, 920 bp, 925 bp, 930 bp, 935 bp, 940 bp, 945 bp, 950 bp, 955 bp, 960 bp, 965 bp, 970 bp, 975 bp, 980 bp, 985 bp, 990 bp, 995 bp, 1000 bp, 1025 bp, 1050 bp, 1075 bp, 1100 bp, 1125 bp, 1150 bp, 1175 bp, 1200 bp, 1225 bp, 1250 bp, 1275 bp, 1300 bp, 1325 bp, 1350 bp, 1375 bp, 1400 bp, 1425 bp, 1450 bp, 1475 bp, 1500 bp, 1525 bp, 1550 bp, 1575 bp, 1600 bp, 1625 bp, 1650 bp, 1675 bp, 1700 bp, 1725 bp, 1750 bp, 1775 bp, 1800 bp, 1825 bp, 1850 bp, 1875 bp, 1900 bp, 1925 bp, 1950 bp, 1975 bp, 2000 bp, 2025 bp, 2050 bp, 2075 bp, 2100 bp, 2125 bp, 2150 bp, 2175 bp, 2200 bp, 2225 bp, 2250 bp, 2275 bp, 2300 bp, 2325 bp, 2350 bp, 2375 bp, 2400 bp, 2425 bp, 2450 bp, 2475 bp, 2500 bp, 2525 bp, 2550 bp, 2575 bp, 2600 bp, 2625 bp, 2650 bp, 2675 bp, 2700 bp, 2725 bp, 2750 bp, 2775 bp, 2800 bp, 2825 bp, 2850 bp, 2875 bp, 2900 bp, 2925 bp, 2950 bp, 2975 bp, 3000 bp, 3025 bp, 3050 bp, 3075 bp, 3100 bp, 3125 bp, 3150 bp, 3175 bp, 3200 bp, 3225 bp, 3250 bp, 3275 bp, 3300 bp, 3325 bp, 3350 bp, 3375 bp, 3400 bp, 3425 bp, 3450 bp, 3475 bp, 3500 bp, 3525 bp, 3550 bp, 3575 bp, 3600 bp, 3625 bp, 3650 bp, 3675 bp, 3700 bp, 3725 bp, 3750 bp, 3775 bp, 3800 bp, 3825 bp, 3850 bp, 3875 bp, 3900 bp, 3925 bp, 3950 bp, 3975 bp, 4000 bp, 4025 bp, 4050 bp, 4075 bp, 4100 bp, 4125 bp, 4150 bp, 4175 bp, 4200 bp, 4225 bp, 4250 bp, 4275 bp, 4300 bp, 4325 bp, 4350 bp, 4375 bp, 4400 bp, 4425 bp, 4450 bp, 4475 bp, 4500 bp, 4525 bp, 4550 bp, 4575 bp, 4600 bp, 4625 bp, 4650 bp, 4675 bp, 4700 bp, 4725 bp, 4750 bp, 4775 bp, 4800 bp, 4825 bp, 4850 bp, 4875 bp, 4900 bp, 4925 bp, 4950 bp, 4975 bp, 5000 bp, 5025 bp, 5050 bp, 5075 bp, 5100 bp, 5125 bp, 5150 bp, 5175 bp, 5200 bp, 5225 bp, 5250 bp, 5275 bp, 5300 bp, 5325 bp, 5350 bp, 5375 bp, 5400 bp, 5425 bp, 5450 bp, 5475 bp, 5500 bp, 5525 bp, 5550 bp, 5575 bp, 5600 bp, 5625 bp, 5650 bp, 5675 bp, 5700 bp, 5725 bp, 5750 bp, 5775 bp, 5800 bp, 5825 bp, 5850 bp, 5875 bp, 5900 bp, 5925 bp, 5950 bp, 5975 bp, 6000 bp, 6025 bp, 6050 bp, 6075 bp, 6100 bp, 6125 bp, 6150 bp, 6175 bp, 6200 bp, 6225 bp, 6250 bp, 6275 bp, 6300 bp, 6325 bp, 6350 bp, 6375 bp, 6400 bp, 6425 bp, 6450 bp, 6475 bp, 6500 bp, 6525 bp, 6550 bp, 6575 bp, 6600 bp, 6625 bp, 6650 bp, 6675 bp, 6700 bp, 6725 bp, 6750 bp, 6775 bp, 6800 bp, 6825 bp, 6850 bp, 6875 bp, 6900 bp, 6925 bp, 6950 bp, 6975 bp, 7000 bp, 7025 bp, 7050 bp, 7075 bp, 7100 bp, 7125 bp, 7150 bp, 7175 bp, 7200 bp, 7225 bp, 7250 bp, 7275 bp, 7300 bp, 7325 bp, 7350 bp, 7375 bp, 7400 bp, 7425 bp, 7450 bp, 7475 bp, 7500 bp, 7525 bp, 7550 bp, 7575 bp, 7600 bp, 7625 bp, 7650 bp, 7675 bp, 7700 bp, 7725 bp, 7750 bp, 7775 bp, 7800 bp, 7825 bp, 7850 bp, 7875 bp, 7900 bp, 7925 bp, 7950 bp, 7975 bp, 8000 bp, 8025 bp, 8050 bp, 8075 bp, 8100 bp, 8125 bp, 8150 bp, 8175 bp, 8200 bp, 8225 bp, 8250 bp, 8275 bp, 8300 bp, 8325 bp, 8350 bp, 8375 bp, 8400 bp, 8425 bp, 8450 bp, 8475 bp, 8500 bp, 8525 bp, 8550 bp, 8575 bp, 8600 bp, 8625 bp, 8650 bp, 8675 bp, 8700 bp, 8725 bp, 8750 bp, 8775 bp, 8800 bp, 8825 bp, 8850 bp, 8875 bp, 8900 bp, 8925 bp, 8950 bp, 8975 bp, 9000 bp, 9025 bp, 9050 bp, 9075 bp, 9100 bp, 9125 bp, 9150 bp, 9175 bp, 9200 bp, 9225 bp, 9250 bp, 9275 bp, 9300 bp, 9325 bp, 9350 bp, 9375 bp, 9400 bp, 9425 bp, 9450 bp, 9475 bp, 9500 bp, 9525 bp, 9550 bp, 9575 bp, 9600 bp, 9625 bp, 9650 bp, 9675 bp, 9700 bp, 9725 bp, 9750 bp, 9775 bp, 9800 bp, 9825 bp, 9850 bp, 9875 bp, 9900 bp, 9925 bp, 9950 bp, 9975 bp, or 10000 bp in length.

In some embodiments, the circular nucleic acid molecule persists in a cell during cell division. In some embodiments, the circular nucleic acid molecule persists in daughter cells after mitosis. In some embodiments, the circular nucleic acid molecule is replicated within a cell and is passed to daughter cells. In some embodiments, the circular nucleic acid molecule comprises a replication element that mediates self-replication of the circular nucleic acid molecule. In some embodiments, the replication element mediates transcription of the circular nucleic acid molecule into a linear nucleic acid molecule that is complementary to the circular nucleic acid molecule (linear complementary). In some embodiments, the linear nucleic acid molecule can be circularized in vivo in cells into a circular nucleic acid molecule. In some embodiments, the nucleic acid molecule can further self-replicate into another circular nucleic acid molecule, which has the same or similar nucleotide sequence as the starting circular nucleic acid molecule. One exemplary self-replication element includes the HDV replication domain (as described by Beeharry et al, Virol, 2014, 450-451:165-173). In some embodiments, a cell passes at least one circular nucleic acid molecule to daughter cells with an efficiency of at least 25%, 50%, 60%, 70%, 80%, 85%, 90%, 95%, or 99%. In some embodiments, a cell undergoing meiosis passes the circular nucleic acid molecule to daughter cells with an efficiency of at least 25%, 50%, 60%, 70%, 80%, 85%, 90%, 95%, or 99%. In some embodiments, a cell undergoing mitosis passes the circular nucleic acid molecule to daughter cells with an efficiency of at least 25%, 50%, 60%, 70%, 80%, 85%, 90%, 95%, or 99%.

Nucleic acid molecules described herein may be circularized in vivo or in vitro according to any known method in the art. For example, in vitro circularization can be done through chemical synthesis, ensuring homogenous 5′- and 3′-ends. As another example, modified group I introns with permuted introns and exons (PIE strategy) can be used. In general, chemical or enzymatic splint ligation strategies are easily adaptable to any sequence of interest. The nucleic acid molecules described herein may be circularized through chemical ligation. Chemical ligation of nucleic acid strands can be done, for example, through cyanogen bromide (BrCN) together with morpholino derivatives such as 2-(N-morpholino)-ethane sulfonic acid (MES) for linking two DNA strands carrying terminal 5′-hydroxyl and 3′-phosphate groups. Chemical ligation strategies are also useful for circularization experiments, provided that the two ends of a linear nucleic acid strand are brought in close proximity. This is typically achieved by an oligonucleotide splint that interacts with the two termini and thus allows circularization.

A number of protocols for enzymatic ligation of synthetic oligonucleotides have been developed. Typically, three different polynucleotide ligases, capable of ligating nicks in single- and/or double-stranded RNA constructs are used: T4 DNA ligase (T4 Dnl), T4 RNA ligase 1 (T4 Rnl 1) and T4 RNA ligase 2 (T4 Rnl 2), all encoded in the genome of bacteriophage T4. They catalyze the formation of a phosphodiester bond between 5′-phosphate (donor) and 3′-hydroxyl (acceptor) end groups in DNA or RNA in an ATP-dependent reaction.

Independent of the chemical or enzymatic preparation of the linear precursor, the 5′-terminus needs to be phosphorylated to provide the functionality required for ligation. Phosphorylation can be performed chemically or enzymatically followed directly by the circularization reaction. In the case of enzymatically prepared RNA substrates, the 5′-terminal triphosphate resulting from the in vitro transcription reaction must be removed prior to phosphorylation by a polynucleotide kinase. To circumvent the two enzymatic steps of de- and rephosphorylation, GMP-primed in vitro transcription by T7 RNA Polymerase is a way for obtaining 5′-monophosphorylated linear RNA substrates, which can be directly used for RNA circularization. Alternatively, an RNA 5′ Pyrophosphohydrolase (RppH) may be used to remove a pyrophosphate from the 5′ end of triphosphorylated RNA to leave a 5′ monophosphate RNA.

When using T4 DNA ligase for circularization, the ligation site may be located within a double-stranded region of the nucleic acid. An oligonucleotide splint can be used to create the required double-stranded region around the ligation site, and in addition to ensure juxtaposition of the 5′-phosphate and 3′-hydroxyl termini in duplex DNA or RNA. Temperature, stoichiometry and splint length can be adjusted for each substrate. T4 RNA ligase 1 can assist in the formation of 3′, 5′-phosphodiester bonds in single-stranded RNA molecules. T4 RNA ligase 1 has different preferences for donor and acceptor nucleotides at the ligation site: A>G≥C>U for the 3′-terminal nucleotide acceptor, and pC>pU>pA>pG for the 5′-terminal nucleotide donor. T4 RNA ligase 1 can be used for circularization in a number of applications with short and long RNAs as substrates. Ligation may be accomplished with the use of T4 RNA ligase 2.

There are a number of other ligases capable of supporting RNA circularization. For example, tRNA ligases from wheat germ and a similar ‘ligase’ from Saccharomyces cerevisiae may be applied for RNA ligation in vitro. Compared with RNA ligases 1 and 2, these ligases accept substrates with 5′-terminal phosphate donors and 2′-terminal phosphate acceptors. The formed internucleotide bond is a 2′-phosphomonoester, 3′, 5′-phosphodiester structure, as appearing in tRNA splicing intermediates in vivo. A recombinant yeast RNA ligase was shown to convert linear introns to their circular counterparts, although notably slower than wild-type Arabidopsis tRNA ligase.

Artificial rolling circle replication and hairpin ribozyme (HPR) methods may be used for circularization. Viroids and other small infectious RNAs replicate via a double rolling circle mechanism. Circular single-stranded DNA templates can be used for in vitro transcription by either T7 or E. coli RNA polymerase resulting in a multimeric repeating RNA sequence harboring the HPR element. Thus, upon transcription, the resulting RNA cleaves itself into monomer-length segments.

Catalytic ribozymes may also be used for circularization. In contrast to the methods of chemical and enzymatic ligation, which can be merely applied in vitro, spontaneous group I catalytic introns allow circular RNA production in vitro and in vivo using the so-called PIE method. Mechanistically, the PIE method includes two transesterification reactions at defined splice sites as occurs in the normal group I intron self-splicing reaction. However, after splicing the normal intron exons are ligated, whereas in the PIE method they are circularized. The first transesterification leads to release of the 3′-terminal sequence (5′-half intron) of the PIE construct. The newly generated free 3′—OH group of the 3′-half exon attacks the 3′-splice site in the second transesterification. This results in circRNA and release of the 3′-half intron. It is also possible to modify group II introns for inverse splicing in vitro generating RNA circles. In this method, a self-splicing group II intron catalyzes the formation of a circular human exon in vitro. RNA circles are formed by inverse splicing, which requires arranging the exons consecutively, positioning the branch point upstream and intronic sequences up- and downstream of the exons. This design allows exon circularization upon two transesterification reactions, excluding any intronic sequences in the circle. An advantage of this strategy, in comparison to group I intron-mediated exon circularization, is the somewhat higher efficiency and complete sequence flexibility (for group I intron-mediated exon circularization the 3′-terminal residue of the 5′-exon must be U). However, based on the group II intron self-splicing mechanism, circRNAs produced via this pathway carry a 2′,5′-phosphodiester at the ligation/circularization site.

### Linear Nucleic Acid Molecules

In some embodiments, the nucleic acid molecule is a linear nucleic acid molecule. In some embodiments, the linear nucleic acid molecule comprises an IRES sequence (e.g., an IRES sequence listed in Table 1). In some embodiments, the linear nucleic acid molecule is a linear RNA molecule. In some embodiments, the linear nucleic acid molecule is a linear DNA molecule. In some embodiments, the linear nucleic acid molecule is a linear hybrid molecule comprising DNA and RNA nucleotides. In some embodiments, a linear nucleic acid molecule includes a nucleic acid molecule capable of circularization but which has not yet circularized and is in a linear configuration. In some embodiments, a linear nucleic acid molecule includes a substantially identical nucleotide sequence as a circular counterpart, as herein disclosed, but has not been joined at the 3′ and 5′ ends.

In some embodiments, the linear nucleic acid molecule is about 500, 1,000, 2,000, 3,000, 4,000, 5,000, or 6,000 nucleotides in size. In some embodiments, the linear nucleic acid molecule is at least 25 nucleotides, at least 50 nucleotides, at least 75 nucleotides, at least 100 nucleotides, at least 125 nucleotides, at least 150 nucleotides, at least 175 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides, at least 400 nucleotides, at least 425 nucleotides, at least 450 nucleotides, at least 475 nucleotides, at least 500 nucleotides, at least 525 nucleotides, at least 550 nucleotides, at least 575 nucleotides, at least 600 nucleotides, at least 625 nucleotides, at least 650 nucleotides, at least 675 nucleotides, at least 700 nucleotides, at least 725 nucleotides, at least 750 nucleotides, at least 775 nucleotides, at least 800 nucleotides, at least 825 nucleotides, at least 850 nucleotides, at least 875 nucleotides, at least 900 nucleotides, at least 925 nucleotides, at least 950 nucleotides, at least 975 nucleotides, at least 1000 nucleotides, at least 1025 nucleotides, at least 1050 nucleotides, at least 1075 nucleotides, at least 1100 nucleotides, at least 1125 nucleotides, at least 1150 nucleotides, at least 1175 nucleotides, at least 1200 nucleotides, at least 1225 nucleotides, at least 1250 nucleotides, at least 1275 nucleotides, at least 1300 nucleotides, at least 1325 nucleotides, at least 1350 nucleotides, at least 1375 nucleotides, at least 1400 nucleotides, at least 1425 nucleotides, at least 1450 nucleotides, at least 1475 nucleotides, at least 1500 nucleotides, at least 1525 nucleotides, at least 1550 nucleotides, at least 1575 nucleotides, at least 1600 nucleotides, at least 1625 nucleotides, at least 1650 nucleotides, at least 1675 nucleotides, at least 1700 nucleotides, at least 1725 nucleotides, at least 1750 nucleotides, at least 1775 nucleotides, at least 1800 nucleotides, at least 1825 nucleotides, at least 1850 nucleotides, at least 1875 nucleotides, at least 1900 nucleotides, at least 1925 nucleotides, at least 1950 nucleotides, at least 1975 nucleotides, at least 2000 nucleotides, at least 2025 nucleotides, at least 2050 nucleotides, at least 2075 nucleotides, at least 2100 nucleotides, at least 2125 nucleotides, at least 2150 nucleotides, at least 2175 nucleotides, at least 2200 nucleotides, at least 2225 nucleotides, at least 2250 nucleotides, at least 2275 nucleotides, at least 2300 nucleotides, at least 2325 nucleotides, at least 2350 nucleotides, at least 2375 nucleotides, at least 2400 nucleotides, at least 2425 nucleotides, at least 2450 nucleotides, at least 2475 nucleotides, at least 2500 nucleotides, at least 2525 nucleotides, at least 2550 nucleotides, at least 2575 nucleotides, at least 2600 nucleotides, at least 2625 nucleotides, at least 2650 nucleotides, at least 2675 nucleotides, at least 2700 nucleotides, at least 2725 nucleotides, at least 2750 nucleotides, at least 2775 nucleotides, at least 2800 nucleotides, at least 2825 nucleotides, at least 2850 nucleotides, at least 2875 nucleotides, at least 2900 nucleotides, at least 2925 nucleotides, at least 2950 nucleotides, at least 2975 nucleotides, at least 3000 nucleotides, at least 3025 nucleotides, at least 3050 nucleotides, at least 3075 nucleotides, at least 3100 nucleotides, at least 3125 nucleotides, at least 3150 nucleotides, at least 3175 nucleotides, at least 3200 nucleotides, at least 3225 nucleotides, at least 3250 nucleotides, at least 3275 nucleotides, at least 3300 nucleotides, at least 3325 nucleotides, at least 3350 nucleotides, at least 3375 nucleotides, at least 3400 nucleotides, at least 3425 nucleotides, at least 3450 nucleotides, at least 3475 nucleotides, at least 3500 nucleotides, at least 3525 nucleotides, at least 3550 nucleotides, at least 3575 nucleotides, at least 3600 nucleotides, at least 3625 nucleotides, at least 3650 nucleotides, at least 3675 nucleotides, at least 3700 nucleotides, at least 3725 nucleotides, at least 3750 nucleotides, at least 3775 nucleotides, at least 3800 nucleotides, at least 3825 nucleotides, at least 3850 nucleotides, at least 3875 nucleotides, at least 3900 nucleotides, at least 3925 nucleotides, at least 3950 nucleotides, at least 3975 nucleotides, at least 4000 nucleotides, at least 4025 nucleotides, at least 4050 nucleotides, at least 4075 nucleotides, at least 4100 nucleotides, at least 4125 nucleotides, at least 4150 nucleotides, at least 4175 nucleotides, at least 4200 nucleotides, at least 4225 nucleotides, at least 4250 nucleotides, at least 4275 nucleotides, at least 4300 nucleotides, at least 4325 nucleotides, at least 4350 nucleotides, at least 4375 nucleotides, at least 4400 nucleotides, at least 4425 nucleotides, at least 4450 nucleotides, at least 4475 nucleotides, at least 4500 nucleotides, at least 4525 nucleotides, at least 4550 nucleotides, at least 4575 nucleotides, at least 4600 nucleotides, at least 4625 nucleotides, at least 4650 nucleotides, at least 4675 nucleotides, at least 4700 nucleotides, at least 4725 nucleotides, at least 4750 nucleotides, at least 4775 nucleotides, at least 4800 nucleotides, at least 4825 nucleotides, at least 4850 nucleotides, at least 4875 nucleotides, at least 4900 nucleotides, at least 4925 nucleotides, at least 4950 nucleotides, at least 4975 nucleotides, at least 5000 nucleotides, at least 5025 nucleotides, at least 5050 nucleotides, at least 5075 nucleotides, at least 5100 nucleotides, at least 5125 nucleotides, at least 5150 nucleotides, at least 5175 nucleotides, at least 5200 nucleotides, at least 5225 nucleotides, at least 5250 nucleotides, at least 5275 nucleotides, at least 5300 nucleotides, at least 5325 nucleotides, at least 5350 nucleotides, at least 5375 nucleotides, at least 5400 nucleotides, at least 5425 nucleotides, at least 5450 nucleotides, at least 5475 nucleotides, at least 5500 nucleotides, at least 5525 nucleotides, at least 5550 nucleotides, at least 5575 nucleotides, at least 5600 nucleotides, at least 5625 nucleotides, at least 5650 nucleotides, at least 5675 nucleotides, at least 5700 nucleotides, at least 5725 nucleotides, at least 5750 nucleotides, at least 5775 nucleotides, at least 5800 nucleotides, at least 5825 nucleotides, at least 5850 nucleotides, at least 5875 nucleotides, at least 5900 nucleotides, at least 5925 nucleotides, at least 5950 nucleotides, at least 5975 nucleotides, or at least 6000 nucleotides.

In some embodiments, the linear nucleic acid molecule is no more than 500 bp, 505 bp, 510 bp, 515 bp, 520 bp, 525 bp, 530 bp, 535 bp, 540 bp, 545 bp, 550 bp, 555 bp, 560 bp, 565 bp, 570 bp, 575 bp, 580 bp, 585 bp, 590 bp, 595 bp, 600 bp, 605 bp, 610 bp, 615 bp, 620 bp, 625 bp, 630 bp, 635 bp, 640 bp, 645 bp, 650 bp, 655 bp, 660 bp, 665 bp, 670 bp, 675 bp, 680 bp, 685 bp, 690 bp, 695 bp, 700 bp, 705 bp, 710 bp, 715 bp, 720 bp, 725 bp, 730 bp, 735 bp, 740 bp, 745 bp, 750 bp, 755 bp, 760 bp, 765 bp, 770 bp, 775 bp, 780 bp, 785 bp, 790 bp, 795 bp, 800 bp, 805 bp, 810 bp, 815 bp, 820 bp, 825 bp, 830 bp, 835 bp, 840 bp, 845 bp, 850 bp, 855 bp, 860 bp, 865 bp, 870 bp, 875 bp, 880 bp, 885 bp, 890 bp, 895 bp, 900 bp, 905 bp, 910 bp, 915 bp, 920 bp, 925 bp, 930 bp, 935 bp, 940 bp, 945 bp, 950 bp, 955 bp, 960 bp, 965 bp, 970 bp, 975 bp, 980 bp, 985 bp, 990 bp, 995 bp, 1000 bp, 1025 bp, 1050 bp, 1075 bp, 1100 bp, 1125 bp, 1150 bp, 1175 bp, 1200 bp, 1225 bp, 1250 bp, 1275 bp, 1300 bp, 1325 bp, 1350 bp, 1375 bp, 1400 bp, 1425 bp, 1450 bp, 1475 bp, 1500 bp, 1525 bp, 1550 bp, 1575 bp, 1600 bp, 1625 bp, 1650 bp, 1675 bp, 1700 bp, 1725 bp, 1750 bp, 1775 bp, 1800 bp, 1825 bp, 1850 bp, 1875 bp, 1900 bp, 1925 bp, 1950 bp, 1975 bp, 2000 bp, 2025 bp, 2050 bp, 2075 bp, 2100 bp, 2125 bp, 2150 bp, 2175 bp, 2200 bp, 2225 bp, 2250 bp, 2275 bp, 2300 bp, 2325 bp, 2350 bp, 2375 bp, 2400 bp, 2425 bp, 2450 bp, 2475 bp, 2500 bp, 2525 bp, 2550 bp, 2575 bp, 2600 bp, 2625 bp, 2650 bp, 2675 bp, 2700 bp, 2725 bp, 2750 bp, 2775 bp, 2800 bp, 2825 bp, 2850 bp, 2875 bp, 2900 bp, 2925 bp, 2950 bp, 2975 bp, 3000 bp, 3025 bp, 3050 bp, 3075 bp, 3100 bp, 3125 bp, 3150 bp, 3175 bp, 3200 bp, 3225 bp, 3250 bp, 3275 bp, 3300 bp, 3325 bp, 3350 bp, 3375 bp, 3400 bp, 3425 bp, 3450 bp, 3475 bp, 3500 bp, 3525 bp, 3550 bp, 3575 bp, 3600 bp, 3625 bp, 3650 bp, 3675 bp, 3700 bp, 3725 bp, 3750 bp, 3775 bp, 3800 bp, 3825 bp, 3850 bp, 3875 bp, 3900 bp, 3925 bp, 3950 bp, 3975 bp, 4000 bp, 4025 bp, 4050 bp, 4075 bp, 4100 bp, 4125 bp, 4150 bp, 4175 bp, 4200 bp, 4225 bp, 4250 bp, 4275 bp, 4300 bp, 4325 bp, 4350 bp, 4375 bp, 4400 bp, 4425 bp, 4450 bp, 4475 bp, 4500 bp, 4525 bp, 4550 bp, 4575 bp, 4600 bp, 4625 bp, 4650 bp, 4675 bp, 4700 bp, 4725 bp, 4750 bp, 4775 bp, 4800 bp, 4825 bp, 4850 bp, 4875 bp, 4900 bp, 4925 bp, 4950 bp, 4975 bp, 5000 bp, 5025 bp, 5050 bp, 5075 bp, 5100 bp, 5125 bp, 5150 bp, 5175 bp, 5200 bp, 5225 bp, 5250 bp, 5275 bp, 5300 bp, 5325 bp, 5350 bp, 5375 bp, 5400 bp, 5425 bp, 5450 bp, 5475 bp, 5500 bp, 5525 bp, 5550 bp, 5575 bp, 5600 bp, 5625 bp, 5650 bp, 5675 bp, 5700 bp, 5725 bp, 5750 bp, 5775 bp, 5800 bp, 5825 bp, 5850 bp, 5875 bp, 5900 bp, 5925 bp, 5950 bp, 5975 bp, 6000 bp, 6025 bp, 6050 bp, 6075 bp, 6100 bp, 6125 bp, 6150 bp, 6175 bp, 6200 bp, 6225 bp, 6250 bp, 6275 bp, 6300 bp, 6325 bp, 6350 bp, 6375 bp, 6400 bp, 6425 bp, 6450 bp, 6475 bp, 6500 bp, 6525 bp, 6550 bp, 6575 bp, 6600 bp, 6625 bp, 6650 bp, 6675 bp, 6700 bp, 6725 bp, 6750 bp, 6775 bp, 6800 bp, 6825 bp, 6850 bp, 6875 bp, 6900 bp, 6925 bp, 6950 bp, 6975 bp, 7000 bp, 7025 bp, 7050 bp, 7075 bp, 7100 bp, 7125 bp, 7150 bp, 7175 bp, 7200 bp, 7225 bp, 7250 bp, 7275 bp, 7300 bp, 7325 bp, 7350 bp, 7375 bp, 7400 bp, 7425 bp, 7450 bp, 7475 bp, 7500 bp, 7525 bp, 7550 bp, 7575 bp, 7600 bp, 7625 bp, 7650 bp, 7675 bp, 7700 bp, 7725 bp, 7750 bp, 7775 bp, 7800 bp, 7825 bp, 7850 bp, 7875 bp, 7900 bp, 7925 bp, 7950 bp, 7975 bp, 8000 bp, 8025 bp, 8050 bp, 8075 bp, 8100 bp, 8125 bp, 8150 bp, 8175 bp, 8200 bp, 8225 bp, 8250 bp, 8275 bp, 8300 bp, 8325 bp, 8350 bp, 8375 bp, 8400 bp, 8425 bp, 8450 bp, 8475 bp, 8500 bp, 8525 bp, 8550 bp, 8575 bp, 8600 bp, 8625 bp, 8650 bp, 8675 bp, 8700 bp, 8725 bp, 8750 bp, 8775 bp, 8800 bp, 8825 bp, 8850 bp, 8875 bp, 8900 bp, 8925 bp, 8950 bp, 8975 bp, 9000 bp, 9025 bp, 9050 bp, 9075 bp, 9100 bp, 9125 bp, 9150 bp, 9175 bp, 9200 bp, 9225 bp, 9250 bp, 9275 bp, 9300 bp, 9325 bp, 9350 bp, 9375 bp, 9400 bp, 9425 bp, 9450 bp, 9475 bp, 9500 bp, 9525 bp, 9550 bp, 9575 bp, 9600 bp, 9625 bp, 9650 bp, 9675 bp, 9700 bp, 9725 bp, 9750 bp, 9775 bp, 9800 bp, 9825 bp, 9850 bp, 9875 bp, 9900 bp, 9925 bp, 9950 bp, 9975 bp, or 10000 bp in length.

### Internal Ribosomal Entry Site (IRES) Sequences

In some embodiments, the nucleic acid molecule disclosed herein comprises an internal ribosomal entry site (IRES) sequence. In some embodiments, the IRES sequence comprises a sequence listed in Table 1. In some embodiments, the nucleic acid molecule includes at least one IRES flanking at least one protein coding sequence (e.g., 2, 3, 4, 5 or more protein coding sequences). In some embodiments, the IRES is located upstream (i.e., the 5′ end) of a protein coding sequence in a nucleic acid molecule.

In some embodiments, the IRES sequence is an IRES sequence of viral origin. In some embodiments, the IRES sequence comprises any one of the sequences set forth in Table 1. In some embodiments, the IRES sequence that is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to any one of IRES sequences described herein. As shown in Table 1, SEQ ID NO for IRES sequences correspond to IRES reference numbers cited in the specification, drawings, and exemplification. For example, SEQ ID NO: 1 corresponds to “IRES 1”, SEQ ID NO: 2 corresponds to “IRES 2”, and so forth.

TABLE 1
IRES sequences

| IRES | |
|---|---|
| No/SEQ | |
| ID NO: | Sequence |
| 1 | TAACACCCGTAGCTTTGAAATACGGCTCGCTCTTTGAATATCCTAGAAACC |
| | TTTGAAGCTCTATATCACCTTTGAACGAAACTCTCCTCTTTGAATCCCCGCC |
| | AGCTCTTTGAATAATCATGACTGCTTTGAAAGTCTGCTTCCTCTTTGAATTA |
| | AATATTTTTCTTTGAATTACCTATTCGGCTTTGAATACTTCTTTCTACTTTGA |
| | ACGTTATTTTGCTCTTTGAATCGGCCTCTTTTCTTTGAATGAAACCATACTC |
| | TTTGAATGACTTTTTAATCTTTGAAATCAAACCTTTACTTTGAATTGAATAT |
| | ATCTCTTTGAAATTTCCGACTCGGACCGATGGTGAG |
| 2 | CCTTTGAAACATGTCCTTTAACACGTTGAACTCGTATATAATTGTTTTCTTT |
| | GAAGTCAAAGCATAGTATGCCTTCTTTTTATATCTTTCAAGCGCTTTGAACT |
| | CAGTGCAGACTCGTTTCTTATCATATGCCGTCTATTAACTTTGAAATTTCCG |
| | ACTCGGACCGATGGTGAG |
| 3 | AGAGTCTCGCATCGTTAGCTAGAGTCTCGTTTTATACCTAAGAGTCTCGTT |
| | ACCTGACTTAGAGTCTCGTTCTATTTTCTAGAGTCTCGCCACTTATTTTAGA |
| | GTCTCGAAGCTTATGTTAGAGTCTCGATTTTGTGGTAAGAGTCTCGATTTCC |
| | GACTCGGACCGATGGTGAG |
| 4 | GACAAGGTGTTACAAACATACGCGGATTCCATAGCAGAATTTATGACACC |
| | CAGTGCCCAGAAGAAGGAGCTGTACCTCCAGAACCACTATGATGCTGTTG |
| | AGGCTGGACTTAATTCAATAGCTGCAGCATGGAAAGACTTGGCCAACAAA |
| | TGCTATGCTTACTCCCGACCCGAA |
| 5 | TGCTTTCGTTCGTTTTTTGTTATTTAATGATTCTTCGTCTTGTAAAACCTTTC |
| | GTTCTGTATATAGACTTCCCCCCTTTCTCTGCTTATTGTTCCTTTCGTTCCAC |
| | GTTATTTTTCTTCCTGTGTACTGCAGTTTACTTCCTTTCGTTCATTTCCGACT |
| | CGGACCGATGGTGAG |
| 6 | TTTATGATTTCAACATGGATACATATTTCAGTTCTTTCTTTTTCTCACTATCT |
| | GAAAATATTAGAGTCTCGACTAAATCTTGTGATTTAGAGTCTCGTGTCTGT |
| | GGGATGCGATAGAGTCTCGGAGCCCTTAATGTGCTTAGAGTCTCGATTTCC |
| | GACTCGGACCGATGGTGAG |
| 7 | TTTATGATTTCAACATGGATACATATTTCAGTTCTTTCTTTTTCTCACTATCT |
| | GAAAATATTAGAGTCTCGGTCTCTCGCCTTATATAAGAGTCTCGACAAGCC |
| | CCTTACTTTAAGAGTCTCGTTTCGTTGTAGGATTTTAGAGTCTCGATTTCCG |
| | ACTCGGACCGATGGTGAG |
| 8 | AGAGTCTCGAAATAAAAGTGAGAGTCTCGATATTATCTTTAGAGTCTCGCA |
| | ATTTTTTCAAGAGTCTCGTTGTTTTTTCAAGAGTCTCGCTCTTTACTTTAGA |
| | GTCTCGAAACTATGCTAAGAGTCTCGATCACATGCATAGAGTCTCGATTTC |
| | CGACTCGGACCGATGGTGAG |
| 9 | TTTATGATTTCAACATGGATACATATTTCAGTTCTTTCTTTTTCTCACTATCT |
| | GAAAATACCGGCCGGGTACACCATTATAATTACGCCTATACTCGAGACCG |
| | GCCGGGTTTGTTCTTCAGACTTTTTTATCTACCCTCACCGGCCGGGATTTCC |
| | GACTCGGACCGATGGTGAG |
| 10 | CTCTTTCGTTCTTCGTATAGATGGGGGTTTTATCTCTCTTATGTTCCCCTTTC |
| | GTTCCATTTCTTAATCGCTAGTCCACTAAATTATTGCTCTACTTTCGTTCTAT |
| | TTATGTAAATGATTCATTTCCTAGACTGATTACACTTTCGTTCATTTCCGAC |
| | TCGGACCGATGGTGAG |
| 11 | TGACACAATTTTGCAATGACATGAAGTTGTTGAATGGAATTAGGGCTGGCA |
| | ATTATACATTTATTCCTACTCCGAAAATTATATGTGCATATGGTACTCCTGA |
| | AATATGTTTGAGAGCCCCTGAGATACGATCATTTGGTAGATATTTACCAAT |
| | TGACAAGGATGAATACTCTG |
| 12 | AGAGTCTCGCATCGTTAGCTAGAGTCTCGTTTTATACCTAAGAGTCTCGTT |
| | ACCTGACTTAGAGTCTCGTTCTATTTTCTAGAGTCTCGCCACTTATTTTAGA |
| | GTCTCGAAGCTTATGTTAGAGTCTCGATTTTGTGGTAAGAGTCTCGATTTCC |
| | GACTCGGACCGATGGTGAG |
| 13 | ATGCTGCTACTATCTAGTTCTATAAGCTTATATAAAGACAGAAATGAAGCA |
| | AGACTGATTTCTTCACAGAATAATGCCGGACTTGAAGATTTTGAAAGGAAA |
| | AAAACCCTTGGAACAGGTTCATTTGGAAGAGTCATGTTGGTAAAACACAA |
| | AGCCACTGAACAGTATTATGCC |
| 14 | GCGGCGCAATACGAATGCCCCATACATCTGATGCGGCGCAATACGAATGC |
| | CCCTCTTACAAAACGCGGCGCAATACGAATGCCCCTGTTCCTTCAAGCGGC |
| | GCAATACGAATGCCCCCTTGCCTAGGTGCGGCGCAATACGAATGCCCCATT |
| | TCCGACTCGGACCGATGGTGAG |
| 15 | TTTATGATTTCAACATGGATACATATTTCAGTTCTTTCTTTTTCTCACTATCT |
| | GAAAATAAGAGTCTCGGGGTATCAATTATCACTGTTCACGATTTATAAGAG |
| | TCTCGGTTTTTTTTATGATTTCCACCTGCATGCTTAAGAGTCTCGATTTCCG |
| | ACTCGGACCGATGGTGAG |
| 16 | CAATAAAGTATTTAAAGCTGGGTATAAAGGAGGGATTTGGGCTTTTGTTGT |
| | GAGCATCACCAACACGTACATCTACGCGTACCTGACATGGCCGCGCCCAG |
| | GGGCAGACCCAAAAAGGACCTTACGATGGAAGACCTAACCGCAAAGATAA |
| | GCCAATTGACTGTGGAGAATCGG |
| 17 | TGCTTTCGTTCGTTTTTTGTTATTTAATGATTCTTCGTCTTGTAAAACCTTTC |
| | GTTCTGTATATAGACTTCCCCCCTTTCTCTGCTTATTGTTCCTTTCGTTCCAC |
| | GTTATTTTTCTTCCTGTGTACTGCAGTTTACTTCCTTTCGTTCATTTCCGACT |
| | CGGACCGATGGTGAG |
| 18 | AGAGTCTCGAAATAAAAGTGAGAGTCTCGATATTATCTTTAGAGTCTCGCA |
| | ATTTTTTCAAGAGTCTCGTTGTTTTTTCAAGAGTCTCGCTCTTTACTTTAGA |
| | GTCTCGAAACTATGCTAAGAGTCTCGATCACATGCATAGAGTCTCGATTTC |
| | CGACTCGGACCGATGGTGAG |
| 19 | GCGGCGCAATACGAATGCCCCAGTCCTTCTCTATCTCGCCTACTAAATGTT |
| | TTATTTATTTTTTTAAATTCCACCTGTCCTATACGATTCTTGCATCCTAGTTT |
| | ACCTCCGTAATTGTTGAAATGTACGCGGCGCAATACGAATGCCCCATTTCC |
| | GACTCGGACCGATGGTGAG |
| 20 | GTACTCCCTTTCTGATTCTTTTAGCTAACTATGCGTAAGCTATTCGATTACT |
| | CCCATCTTTACAATTACTAGTTTCATCTGGCCCCTGCATAATATACTCCCAG |
| | TAACCAATCTGCAAACACACCAAATAAGTTAAAATCCCTACTCCCATTTCC |
| | GACTCGGACCGATGGTGAG |
| 21 | TGAGCTGATCGTTGTTGTTATTCTTCAATTTCTGCAGGTAACTTAGGAAGCG |
| | TTTCTTGAGCTTCCCTGGGATGAGCGTTTGGGAGAGCTGATTCTGCAGCCC |
| | AGAGAGTAGTCTCAGGGCATCCTCTGGAGCCTGACCTGTGACCGTCGCATC |
| | ATAGACCGCCAGTAGACCTGGGAGCAGATTCACCGCCGCGGCCGTCTCCTT |
| | TAAGGTGCTGTG |
| 22 | TTTATGATTTCAACATGGATACATATTTCAGTTCTTTCTTTTTCTCACTATCT |
| | GAAAATAGTCCGGCCGGGTTCTCTCAAACTCGTTACCGGCCGGGGAGTTTT |
| | CTACCTATATCCGGCCGGGATATTCTTGGAAGCTTTCCGGCCGGGATTTCC |
| | GACTCGGACCGATGGTGAG |
| 23 | GTTTCCACTGCTGCTCTGAGGGCACAAGAGGCAGGATTTGGAATTCCTGGA |
| | GAACTGTCTTCGTGAGAAGCTGGAAATATTTCTTTCAATTCCATCTCTTAGT |
| | TTTCCATTTTGTTGTGTTTCAGAGGAACATCAAGAAACC |
| 24 | CCGGCGGGTATTCTTATATTCCGGCGGGTTTATACATCGCCCGGCGGGTAA |
| | GTGTTTCTTCCGGCGGGTTTGCTTATGTTCCGGCGGGTCATGACTTTCCCCG |
| | GCGGGTGACATCCTGTTCCGGCGGGTTTTTCATATATCCGGCGGGTATTTC |
| | CGACTCGGACCGATGGTGAG |
| 25 | TTTATGATTTCAACATGGATACATATTTCAGTTCTTTCTTTTTCTCACTATCT |
| | GAAAATAGAAGCTGCCCGGCGGGTCAT |
| | GGCGCCATTCAGTTAATTCCCTCCTGTTCTCGCGTTTTATTACTCTTTGAAG |
| | CTGCCCGGCGGGTCATGATTTCCGACTCGGACCGATGGTGAG |
| 26 | TTTTCTTTGAATCTTTTGGTTTCTCTTTGAACCCTCTTGTCTTTCTTTGAATT |
| | CTTTATAATGTCTTTGAAAAAGCTTCTTTGTCTTTGAATTCATTTCGCTGTCT |
| | TTGAAGTCATTAAAGCCGCTTTGAATTTTAATCCGAACCTTTGAATTTCTAC |
| | CAATCGCTTTGAAATCTTATTTACTGCTTTGAATTTTCTTTTTCCTCTTTGAA |
| | TTCTGTGTTTTATCTTTGAAGTGGGCATTATGCCTTTGAACAGACTCTAGCA |
| | ACTTTGAATTCTGCCAATGGTCTTTGAATGCGCATATTTTCCTTTGAATTCT |
| | ATTTATTTACTTTGAATCCCTTAACAGAGCTTTGAAAGCATACCCCAATCTT |
| | TGAATTTATATTTTTTTCTTTGAACACAGTATCTTTTCTTTGAAATATCAAA |
| | GGCCCCTTTGAATTCCCCCTTTATACTTTGAACGTCTTTTACAACCTTTGAA |
| | ACTCACATCTTATCTTTGAACCAACGCTCTTAACTTTGAATCTTCTATCAGA |
| | TCTTTGAATTTGTCCTTACCCCTTTGAAACGATCCACCAATCTTTGAATTTG |
| | TCTGCCGATCTTTGAATTTCACGATCGGTCTTTGAAGAGTAGTCACTTGCTT |
| | TGAATATATGAGCTCCTCTTTGAACATAGATCATTTTCTTTGAAATTTCCGA |
| | CTCGGACCGATGGTGAG |
| 27 | GGGACTATGTTTTGAAACTGACTTCCAGAGCTCCACTGCCTCCCTGCACCA |
| | TGCCCTACAACTTCTGCCTGCCCAGTAATGTGCCTGCACCCAGCTGCTCCTC |
| | CCGGCCCTGTGTGCCCCCCAGCTGCCACGGCTACACCCTGCCCGGGGCCTG |
| | CAACATCCCTGCCAATGTGA |
| 28 | ATACGTATTGGGCAATTAAAAATAATAATTAGGCCTAGGGAACAAATCCC |
| | TCTCAGCGAAGGCCGAAAAGAGGCTAGCCATGCCCTTAGTAGGACTAGCA |
| | TAATGAGGGGGGTAGCAACAGTGGTGAGTTCGTTGGATGGCTTAAGCCCT |
| | GAGTACAGGGTAGTCGTCAGTGGTTCGACGCCTTGGAATAAAGGTCTCGA |
| | GATGCCACGTGGACGAGGGCATGCCCAAAGCACATCTTAACCTGAGCGGG |
| | GGTCGCCCAGGTAAAAGCAGTTTTAACCGACTGTTACGAATACAGCCTGAT |
| | AGGGTGCTGCAGAGGCCCACTGTATTGCTACTAAAAATCTCTGCTGTACAT |
| | GGCACATGGAG |
| 29 | GTATACGAGAATTAGAAAAGGCACTCGTATACGTATTGGGCAATTAAAAA |
| | TAATAATTAGGCCTAGGGAACAAATCCCTCTCAGCGAAGGCCGAAAAGAG |
| | GCTAGCCATGCCCTTAGTAGGACTAGCATAATGAGGGGGGTAGCAACAGT |
| | GGTGAGTTCGTTGGATGGCTTAAGCCCTGAGTACAGGGTAGTCGTCAGTGG |
| | TTCGACGCCTTGGAATAAAGGTCTCGAGATGCCACGTGGACGAGGGCATG |
| | CCCAAAGCACATCTTAACCTGAGCGGGGGTCGCCCAGGTAAAAGCAGTTT |
| | TAACCGACTGTTACGAATACAGCCTGATAGGGTGCTGCAGAGGCCCACTGT |
| | ATTGCTACTAAAAATCTCTGCTGTACATGGCAC |
| 30 | GTATACGAGGTTAGTTCATTCTCGTATACACGATTGGACAAATCAAAATTA |
| | TAATTTGGTTCAGGGCCTCCCTCCAGCGACGGCCGAACTGGGCTAGCCATG |
| | CCCATAGTAGGACTAGCAAAACGGAGGGACTAGCCATAGTGGCGAGCTCC |
| | CTGGGTGGTCTAAGTCCTGAGTACAGGACAGTCGTCAGTAGTTCGACGTGA |
| | GCAGAAGCCCACCTCGAGATGCTACGTGGACGAGGGCATGCCAAGACACA |
| | CCTTAACCCTAGCGGGGGTCGCTAGGGTGAAATCACACCACGTGATGGGA |
| | GTACGACCTGATAGGGCGCTGCAGAGGCCCACTATTAGGCTAGTATAAAA |
| | ATCTCTGCTGTACATGGCAC |
| 31 | ATTGCTTTTCTTCCAACTAAACCCCTATTGATGTGTTGGTCGTTTGCCAATC |
| | GGAGGGCGACAGGCACAGGTCGCTCCGAGTTCTAGTAGTGTGGGAACTTG |
| | TTACTACTGATGAAACGAGGTAGTGACACTCAGTACCTGCGAACGAGGTC |
| | GGGGCCCTCCCTTCTTCCTTCACCCAACTTTCACTTTTCGTTCCACTTTAGC |
| | AGGGGTCTTCTTTCTATCCCCCTGGCGGCATTGGAACTAGCCGTCGCGTCTT |
| | AACGCGCAGCCCTGAAGGCCCCACACCTTGTGGATCTTGCCGTGGGTATGT |
| | TTCTGGCATGTGTTTCTCAAGCCTGCAACCGAAGCCGAACAGCCACATGAA |
| | CAGTTTGAGCGTGGTAGCGCTGTGTGAGTTGGCGGTGGATCCCCCTCGTGG |
| | TAACACGAGCCCCCGTGGCCAAAAGCCCAGTGTTTACAGCACCTCTCACAT |
| | CCAGGACGACCCCATCCTGGCGCTCACTCTTAGTAGTATGGCTTAGTACGC |
| | ATTAGGTGGTAAGCCGAGCTCTCCCTCGGCCTTGTTCTGAATGCACACATG |
| | TCTAGGGGCTAAGGATGTCCTACAGGTACCCGCACGTAACCTTCAGAGAGT |
| | GCGGATCTGAGTAGGAGACCGTGGTGCACTGCTTTACAGATGCAGCCCGG |
| | TTTAAAAAGCGTCTATGCCCCTACAGGGTAGCGGTGGGCCGCGCCCTTTCC |
| | TTTTAAAACTACTTGTTCTATGGTGACAATGGCAGGAAACATGAT |
| 32 | TTAAAACAGCCTGTGGGTTGCACCCACCCACAGGGCCCACTGGGCGCTAG |
| | CACTCTGGTACTGAGGTACCTTTGTGCGCCTGTTTTTACTCCCCTTCCCCCG |
| | AAGTAACTTAGAAGCTGTAAATCAACGATCAATAGCAGGTGTGGCACACC |
| | AGTCATACCTTGATCAAGCACTTCTGTTTCCCCGGACTGAGTATCAATAGG |
| | CTGCTCGCGCGGCTGAAGGAGAAAACGTTCGTTACCCGACCAACTACTTCG |
| | AGAAGCTTAGTACCACCATGAACGAGGCAGGGTGTTTCGCTCAGCACAAC |
| | CCCAGTGTAGATCAGGCTGATGAGTCACTGCAACCCCCATGGGCGACCAT |
| | GGCAGTGGCTGCGTTGGCGGCCTGCCCATGGAGAAATCCATGGGACGCTC |
| | TAATTCTGACATGGTGTGAAGAGCCTATTGAGCTAGCTGGTAGTCCTCCGG |
| | CCCCTGAATGCGGCTAATCCTAACTGCGGAGCACATGCTCACAAACCAGTG |
| | GGTGGTGTGTCGTAACGGGCAACTCTGCAGCGGAACCGACTACTTTGGGTG |
| | TCCGTGTTTCCTTTTATTCCTATATTGGCTGCTTATGGTGACAATCAAAAAG |
| | TTGTTACCATATAGCTATTGGATTGGCCATCCGGTGTGCAACAGGGCAATT |
| | GTTTACCTATTTATTGGTTTTGTACCATTATCACTGAAGTCTGTGATCACTC |
| | TCAAATTCATTTTGACCCTCAACACAATCAAACATG |
| 33 | TTAAAACAGCTCTGGGGTTGTTCCCACCCCAGAGGCCCACGTGGCGGCTAG |
| | TACACTGGTATCACGGTACCTTTGTACGCCTGTTTTATACTCCCTCCCCCGC |
| | AACTTAGAAGCATACAATTCAAGCTCAATAGGAGGGGGTGCAAGCCAGCG |
| | CCTCCGTGGGCAAGCACTACTGTTTCCCCGGTGAGGCCGCATAGACTGTTC |
| | CCACGGTTGAAAGTGGCCGATCCGTTATCCGCTCATGTACTTCGAGAAGCC |
| | TAGTATCGCTCTGGAATCTTCGACGCGTTGCGCTCAGCACTCAACCCCGGA |
| | GTGTAGCTTGGGCCGATGAGTCTGGACAGTCCCCACTGGCGACAGTGGTCC |
| | AGGCTGCGCTGGCGGCCCACCTGTGGCCCAAAGCCACGGGACGCTAGTTG |
| | TGAACAGGGTGTGAAGAGCCTATTGAGCTACATGAGAGTCCTCCGGCCCCT |
| | GAATGCGGCTAATCCTAACCATGGAGCAGGCAGCTGCAACCCAGCAGCCA |
| | GCCTGTCGTAACGCGCAAGTCCGTGGCGGAACCGACTACTTTGGGTGTCCG |
| | TGTTTCCTTTTATTCTTGAATGGCTGCTTATGGTGACAATCATAGATTGTTA |
| | TCATAAAGCGAGTTGGATTGGCCATCCAGTGTGAATCAGATTAATTACTCC |
| | CTTGTTTGTTGGATCCACTCCCGAAACGTTTTACTCCTTAACTTATTGAAAT |
| | TGTTTGAAGACAGGATTTCAGTGTCACA |
| 34 | GCTAGGAATGCTCCTGGAGCACCCCCCCTAGCAGGGCGTGGGGGATTTCCC |
| | CTGCCCGTCTGCAGAAGGGTGGAGCCAACCACCTTAGTATGTAGGCGGCG |
| | GGACTCATGACGCTCGCGTGATGACAAGCGCCAAGCTTGACTTGGATGGC |
| | CCTGATGGGCGTTCATGGGTTCGGTGGTGGTGGCGCTTTAGGCAGCCTCCA |
| | CGCCCACCACCTCCCAGATAGAGCGGCGGCACTGTAGGGAAGACCGGGGA |
| | CCGGTCACTACCAAGGACGCAGACCTCTTTTTGAGTATCACGCCTCCGGAA |
| | GTAGTTGGGCAAGCCCACCTATATGTGTTGGGATGGTTGGGGTTAGCCATC |
| | CATACCGTACTGCCTGATAGGGTCCTTGCGAGGGGATCTGGGAGTCTCGTA |
| | GACCGTAGCAC |
| 35 | GACTAAGTGTGAACTTGCCTCTCTCAACAAAAAGCCACCGACATTAAGAG |
| | AGAGAGTATTGCTGCTTAGTTAACTGCAGGCCCTATTTAGGGTTACCGCCC |
| | AGGATCTGCAACAGCATTCCTGTATCATCCAGGGCACCGGTGGACTTGGTG |
| | AGGATTGAGTTGACCTCATCATTAGAGACCAGACTC |
| 36 | ATCCAGGTTGTTCCCACCTGGATTTCCCACAGGGAGTGGTACTCTGTTATT |
| | ACGGTAACTTTGTACGCCAGTTTTATCTCCCTTCCCCCATGTAACTTAGAAG |
| | TTTTTCACAAAGACCAATAGCCGGTAATCAGCCAGATTACTGAAGGTCAAG |
| | CACTTCTGTTTCCCCGGTCAATGTTGATATGCTCCAACAGGGCAAAAACAA |
| | CTGCGATCGTTAACCGCAAAGCGCCTACGCAAAGCTTAGTAGCATCTTTGA |
| | AATCGTTTGGCTGGTCGATCCGCCATTTCCCCTGGTAGACCTGGCAGATGA |
| | GGCTAGAAATACCCCACTGGCGACAGTGTTCTAGCCTGCGTGGCTGCCTGC |
| | ACACCCTATGGGTGTGAAGCCAAACAATGGACAAGGTGTGAAGAGCCCCG |
| | TGTGCTCGCTTTGAGTCCTCCGGCCCCTGAATGTGGCTAACCTTAACCCTGC |
| | AGCTAGAGCACGTAACCCAATGTGTATCTAGTCGTAATGAGCAATTGCGG |
| | GATGGGACCAACTACTTTGGGTGTCCGTGTTTCACTTTTTCCTTTATATTTG |
| | CTTATGGTGACAATATATACAATATATATATTGGCACCATGG |
| 37 | CAAAATTGCGTGCGAGAAAGCACGCAAATCAAAGTCTAGTGCGTAATTCA |
| | CTCACTACCGGCGTAATTTGTGGTTATGCTATTGCGTTGAGAGTGTTGTGG |
| | GCGTAGTACGGGGGCTTTTGGTTTGTGTGTGATTAATATGCATTCCCAGTTT |
| | TGTTTAGTTTTAGATTACTGATTTATTTTTCGAACTACCCGAATTTATTTAG |
| | GCTCTTCGAGAAATAATGATGAACTGTCCTCAACAAGTATGAAAAGCAATT |
| | ATTTGTGAAGTTACTTGTTGTTGTAAGATTATTGACCTCTTAGATTTTTCTA |
| | AGTTGTAATGCTTTGTTTTCTGATTGACTAGATTATGAATCCAATTAAAAG |
| | GAGTAGTGGTCTAATATAGTCTGTGTGACCTGCAGGCATTTTGTGAAAAGG |
| | GTAAAGTATGAAAGCTACTCTCAGAAAAGTACTTATGTATTGATGAGAGCC |
| | TTAAAATGACTTTATATTCACAAAACTGCTGGAAGACAATGATCTGGGGTA |
| | TCACATTCCCTCTTAGGTTAAGTTTCGCACTACTAGGAATTTTTGCGAAAAT |
| | TCTTAATTCCATTCTTATGGTGTAGTGTTTGTTCTTTTCAATTGGGGCGTAT |
| | CTCTCTTTCGCAGAGACCCTGGCCCAACCCTGAAATGAATAAAATTTTACA |
| | AAAATTCATCGAAAATCGACCTTCTAG |
| 38 | AGCAGGTTTCCCCAACTGACACAAAACGTGCAACTTGAAACTCCGCCTGGT |
| | CTTTCCAGGTCTAGAGGGGTAACACTTTGTACTGTGTTTGGCTCCACGCTC |
| | GATCCACTGGCGAGTGTTAGTAACAGCACTGTTGCTTCGTAGCGGAGCATG |
| | ACGGCCGTGGGAACTCCTCCTTGGTAACAAGGACCCACGGGGCCAAAAGC |
| | CACGCCCACACGGGCCCGTCATGTGTGCAACCCCAGCACGGCGACTTTACT |
| | GCGAAACCCACTTTAAAGTGACATTGAAACTGGTACCCACACACTGGTGA |
| | CAGGCTAAGGATGCCCTTCAGGTACCCCGAGGTAACACGCGACACTCGGG |
| | ATCTGAGAAGGGGACTGGGGCTTCTATAAAAGCGCTCGGTTTAAAAAGCT |
| | TCTATGCCTGAATAGGTGACCGGAGGTCGGCACCTTTCCTTTACAATTAAT |
| | GACCCT |
| 39 | GCCAGCCCCCTGATGGGGGCGACACTCCACCATGAATCACTCCCCTGTGAG |
| | GAACTACTGTCTTCACGCAGAAAGCGTCTAGCCATGGCGTTAGTATGAGTG |
| | TCGTGCAGCCTCCAGGACCCCCCCTCCCGGGAGAGCCATAGTGGTCTGCGG |
| | AACCGGTGAGTACACCGGAATTGCCAGGACGACCGGGTCCTTTCTTGGATA |
| | AACCCGCTCAATGCCTGGAGATTTGGGCGTGCCCCCGCAAGACTGCTAGCC |
| | GAGTAGTGTTGGGTCGCGAAAGGCCTTGTGGTACTGCCTGATAGGGTGCTT |
| | GCGAGTGCCCCGGGAGGTCTCGTAGACCGTGCACCATGAGCACGAATCCT |
| | AAACCTCAAAGAAAAACCAAACGTAAC |
| 40 | TTTGAAAGAGGCCTCCGGAGTGTCCGGAGGCTCTCTTTCGACCCAACCCAT |
| | ACTGGGGGGTGTGTGGGACCGTACCTGGAGTGCACGGTATATATGCATTCC |
| | CGCATGGCAAGGGCGTGCTACCTTGCCCCTTGACGCATGGTATGCGTCATC |
| | ATTTGCCTTGGTTAAGCCCCATAGAAACGAGGCGTCACGTGCCGAAAATCC |
| | CTTTGCGTTTCACAGAACCATCCTAACCATGGGTGTAGTATGGGAATCGTG |
| | TATGGGGATGATTAGGATCTCTCGTAGAGGGATAGGTGTGCCATTCAAATC |
| | CAGGGAGTACTCTGGCTCTGACATTGGGACATTTGATGTAACCGGACCTGG |
| | TTCAGTATCCGGGTTGTCCTGTATTGTTACGGTGTATCCGTCTTGGCACACT |
| | GAAAGGGTATTTTTGGGTAATCCTTTCCTACTGCCTGATAGGGTGGCGTGC |
| | CCGGCCACGAGAGATTAAGGGTAGCAATTTAAAC |
| 41 | GATAAAAGAACCTATAATCCCTTCGCACACCGCGTCACACCGCGCTATATG |
| | CTGCTCATTAGGAATTACGGCTCCTTTTTTGTGGATACAATCTCTTGTATAC |
| | GATATACTTATTGTTAATTTCATTGACCTTTACGCAATCCTGCGTAAATGCT |
| | GGTATAGGGTGTACTTCGGATTTCCGAGCCTATATTGGTTTTGAAAGGACC |
| | TTTAAGTCCCTACTATACTACATTGTACTAGCGTAGGCCACGTAGGCCCGT |
| | AAGATATTATAACTATTTTATTATATTTTATTCACCCCCCACATTAATCCCA |
| | GTTAAAGCTTTATAACTATAAGTAAGCCGTGCCGAAACGTTAATCGGTCGC |
| | TAGTTGCGTAACAACTGTTAGTTTAATTTTCCAAAATTTATTTTTCACAATT |
| | TTTAGTTAAGATTTTAGCTTGCCTTAAGCAGTCTTTATATCTTCTGTATATT |
| | ATTTTAAAGTTTATAGGAGCAAAGTTCGCTTTACTCGCAATAGCTATTTTAT |
| | TTATTTTAGGAATATTATCACCTCGTAATTATTTAATTATAACATTAGCTTT |
| | ATCTATTTATA |
| 42 | TCGTTATATTTAGAATTAGAATGTTTCTTAGCGGTCGTGTAGTTATTTTTAT |
| | GTCATAAGTGGATAATTTGTTAGCTCCTATAACAAAAGTCTGTTGCTTGTG |
| | TTTCACATTTTGGATTTCCTAATATAATGTTCTCTTTTTAGAAAAGGTGGAC |
| | AAGTCCTATTTTCAAGAGA |
| 43 | GCAAAAATGTGATCTTGCTTGTAAATACAATTTTGAGAGGTTAATAAATTA |
| | CAAGTAGTGCTATTTTTGTATTTAGGTTAGCTATTTAGCTTTACGTTCCAGG |
| | ATGCCTAGTGGCAGCCCCACAATATCCAGGAAGCCCTCTCTGCGGTTTTTC |
| | AGATTAGGTAGTCGAAAAACCTAAGAAATTTACCTGCTACATTTCAA |
| 44 | TTCGTTTGCTTTTTGTAGTATAATTAAATATTTGTCAGATAAGAGATTGTTT |
| | AGAGATTTGTTCTTTGTTTGATA |
| 51 | TTAAAACAGCCTGTGGGTTGATCCCACCCACAGGCCCATTGGGCGCTAGCA |
| | CTCTGGTATCACGGTACCTTTGTGCGCCTGTTTTATACCCCCTCCCCCAACT |
| | GTAACTTAGAAGTAACACACACCGATCAACAGTCAGCGTGGCACACCAGC |
| | CACGTTTTGATCAAGCACTTCTGTTACCCCGGACTGAGTATCAATAGACTG |
| | CTCACGCGGTTGAAGGAGAAAGCGTTCGTTATCCGGCCAACTACTTCGAAA |
| | AACCTAGTAACACCGTGGAAGTTGCAGAGTGTTTCGCTCAGCACTACCCCA |
| | GTGTAGATCAGGTCGATGAGTCACCGCATTCCCCACGGGCGACCGTGGCG |
| | GTGGCTGCGTTGGCGGCCTGCCCATGGGGAAACCCATGGGACGCTCTAAT |
| | ACAGACATGGTGCGAAGAGTCTATTGAGCTAGTTGGTAGTCCTCCGGCCCC |
| | TGAATGCGGCTAATCCTAACTGCGGAGCACACACCCTCAAGCCAGAGGGC |
| | AGTGTGTCGTAACGGGCAACTCTGCAGCGGAACCGACTACTTTGGGTGTCC |
| | GTGTTTCATTTTATTCCTATACTGGCTGCTTATGGTGACAATTGAGAGATCG |
| | TTACCATATAGCTATTGGATTGGCCATCCGGTGACTAATAGAGCTATTATA |
| | TATCCCTTTGTTGGGTTTATACCACTTAGCTTGAAAGAGGTTAAAACATTA |
| | CAATTCATTGTTAAGTTGAATACAGCAAA |
| 52 | AAGCAGGTTTCCCCAACTGACACAAACCGTGCAACTTGAAACTCCGCCTGG |
| | TCTTTCCAGGTCTAGAGGGGTGACACTTTGTACTGTGCTTGACTCCACGCTC |
| | GGTCCACTAGCGGGTGTTAGTAACAGCACTGTTGTTTCGTAGCGGAGCATG |
| | ATGGCCGTGGGAACTCCGCCTTGGTAACAAGGACCCACGGGGCCGAAAGC |
| | CACGTCCTAACGGACCCATCATGTGTGCAACCCCAGCACAACAACTTTTCT |
| | GTGAAACCTACCTTAAGGTGACGCTGATACTGGTACTCAGACACTGGTGAC |
| | AGGCTAAGGATGCCCTTCAGGTACCCCGAGGTAACACGCGACACTCGGGA |
| | TCTGAGAAGGGGACTAGGGCTTCTGTAAAAGTGCCTAGTTTAAAAAGCTTC |
| | TATGCCTGAATAGGTGACCGGAGGCCGGCACCTTTCCCTTTACAACTACTG |
| | ACTA |
| 53 | TTAAAACAGCCTGTGGGTTGATCCCACCCACAGGGCCCATTGGGCGCTAGC |
| | ACTCTGGTATCACGGTACCTTTGTGCGCCTGTTTTATACCCCCTCCCCCAAC |
| | TGTAACTTAGAAGTAACACACACCGATCAACAGTCAGCGTGGCACACCAG |
| | CCACGTTTTGATCAAGCACTTCTGTTACCCCGGACTGAGTATCAATAGACT |
| | GCTCACGCGGTTGAAGGAGAAAGCGTTCGTTATCCGGCCAACTACTTCGAA |
| | AAACCTAGTAACACCGTGGAAGTTGCAGAGTGTTTCGCTCAGCACTACCCC |
| | AGTGTAGATCAGGTCGATGAGTCACCGCATTCCCCACGGGCGACCGTGGC |
| | GGTGGCTGCGTTGGCGGCCTGCCCATGGGGAAACCCATGGGACGCTCTAA |
| | TACAGACATGGTGCGAAGAGTCTATTGAGCTAGTTGGTAGTCCTCCGGCCC |
| | CTGAATGCGGCTAATCCTAACTGCGGAGCACACACCCTCAAGCCAGAGGG |
| | CAGTGTGTCGTAACGGGCAACTCTGCAGCGGAACCGACTACTTTGGGTGTC |
| | CGTGTTTCATTTTATTCCTATACTGGCTGCTTATGGTGACAATTGAGAGATT |
| | GTTACCATATAGCTATTGGATTGGCCATCCGGTGACCAATAGAGCTATTAT |
| | ATATCTCTTTGTTGGGTTTATACCACTTAGCTTGAAAGAGGTTAAAACATT |
| | ACAATTCATTGTTAAGTTGAATACAGCAAAATGGGAGC |
| 54 | TTGCCAGTCTGCTCGATATCGCAGGCTGGGTCCGTGACTACCCACTCCCCC |
| | TTTCAACGTGAAGGCTACGATAGTGCCAGGGCGGGTACTGCCGTAAGTGC |
| | CACCCCAAACAACAACAACAAAACAAACTCCCCCTCCCCCCCCTTACTATA |
| | CTGGCCGAAGCCACTTGGAATAAGGCCGGTGTGCGTTTGTCTACATGCTAT |
| | TTTCTACCGCATTACCGTCTTATGGTAATGTGAGGGTCCAGAACCTGACCC |
| | TGTCTTCTTGACGAACACTCCTAGGGGTCTTTCCCCTCTCGACAAAGGAGT |
| | GTAAGGTCTGTTGAATGTCGTGAAGGAAGCAGTTCCTCTGGAAGCTTCTTA |
| | AAGACAAACAACGTCTGTAGCGACCCTTTGCAGGCAGCGGAACCCCCCAC |
| | CTGGTGACAGGTGCCTCTGCGGCCAAAAGCCACGTGTATAAGATACACCT |
| | GCAAAGGCGGCACAACCCCAGTGCCACGTTGTGAGTTGGATAGTTGTGGA |
| | AAGAGTCAAATGGCTCTCCTCAAGCGTATTCAACAAGGGGCTGAAGGATG |
| | CCCAGAAGGTACCCCATTGTATGGGATCTGATCTGGGGCCTCGGTGCACGT |
| | GCTTTACACGTGTTGAGTCGAGGTGAAAAAACGTCTAGGCCCCCCGAACC |
| | ACGGGGACGTGGTTTTCCTTTGAAAACCACGATTACAAT |

### Intron Sequences

In certain aspects, the nucleic acid molecules disclosed herein include one or more self-splicing intron sequence(s) (“intron sequence”) (e.g., one or more intron sequence(s) disclosed herein). In some embodiments, the nucleic acid molecule includes a 5′ intron sequence. In some embodiments, the nucleic acid molecule includes an initial intron sequence. In some embodiments, the nucleic acid molecule includes an initial intron sequence listed in Table 3. In some embodiments, the nucleic acid molecule includes a corresponding intron sequence. In some embodiments, the nucleic acid molecule includes a corresponding′ intron sequence listed in Table 3. In some embodiments, the nucleic acid molecule includes an initial intron sequence and a corresponding intron sequence (e.g., any initial intron sequence and a corresponding intron sequence listed in Table 2). In some embodiments, the initial intron sequence is located within the 5′ end of the nucleic acid molecule and the corresponding intron sequence is located within the 3′ end of the nucleic acid molecule. In some embodiments, the initial intron sequence is located within the 3′ end of the nucleic acid molecule and the corresponding intron sequence is located within the 5′ end of the nucleic acid molecule. In some embodiments, the initial intron sequence is a 5′ intron sequence or a 3′ intron sequence. In some embodiments, the corresponding intron sequence is a 5′ intron sequence or a 3′ intron sequence. In some embodiments, the initial intron sequence is a 5′ intron sequence and the corresponding intron sequence is a 3′ intron sequence. In other embodiments, the corresponding intron sequence is a 5′ intron sequence and the initial intron sequence is a 3′ intron sequence. In some embodiments, the nucleic acid molecules comprise any one of the novel 5′ intron and/or 3′ intron sequences herein disclosed and any one or more of the novel IRES sequences disclosed herein. In some embodiments, the nucleic acid molecules comprise any one of the novel 5′ intron and/or 3′ intron sequences herein disclosed and any one or more IRES sequences known in the art.

These introns possess ribozymatic activity which allows them to self-splice from their host transcripts. In some embodiments, nucleic acid molecules provided herein, which contain split group I introns, permuted so that the donor site is 3′ of the acceptor site, are self-circularizing, and such constructs can be used to produce a protein or polypeptide. In some embodiments, the nucleic acid molecule includes a self-circularizing intron, e.g., a 5′ and 3′ splice junction, or a self-circularizing catalytic intron such as a Group I, Group II, or Group III introns. In some embodiments, the nucleic acid molecules are not self-circularizing.

In one embodiment, either the 5′- or 3′-end of the nucleic acid molecule can encode a ligase ribozyme sequence such that during in vitro transcription, the resultant linear or circular polyribonucleotide includes an active ribozyme sequence capable of ligating the 5′-end to the 3′-end. In some embodiments, the introns are derived from organisms across the animal kingdom (e.g., mammals, reptiles, birds, fish, birds). In some embodiments, the introns are derived from viruses. In certain embodiments, the introns are derived from non-animal organisms (e.g., fungi, plants). In certain embodiments, the introns are of synthetic origin.

In some embodiments, the nucleic acid molecule includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in cis-sequence elements proximal to back splice events such as sequences in the 200 bp preceding (upstream of) or following (downstream from) a back splice site with flanking exons. In some embodiments, the nucleic acid molecule includes at least one repetitive nucleotide sequence described elsewhere herein as an internal splicing element. In such embodiments, the repetitive nucleotide sequence may include repeated sequences from the Alu family of introns.

In some embodiments, the nucleic acid molecule further comprises an initial intron sequence and a corresponding intron sequence. In some embodiments, the initial intron sequence is located within the 5′ end on the nucleic acid molecule and the corresponding intron sequence is located within the 3′ end of the nucleic acid molecule. In some embodiments, the initial intron sequence is located within the 3′ end on the nucleic acid molecule and the corresponding intron sequence is located within the 5′ end of the nucleic acid molecule. Therefore, in some embodiments, the initial intron sequence is a 5′ intron sequence or a 3′ intron sequence. In some embodiments, the corresponding intron sequence is a 5′ intron sequence or a 3′ intron sequence. In some embodiments, the initial intron sequence is a 5′ intron sequence and the corresponding intron sequence is a 3′ intron sequence. In other embodiments, the corresponding intron sequence is a 5′ intron sequence and the initial intron sequence is a 3′ intron sequence.

In some embodiments, the initial intron sequence is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of the sequences set forth in Table 3. In some embodiments, the corresponding intron sequence is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of the sequences set forth in Table 3. Table 3 lists both initial intron sequences and corresponding intron sequences. In some embodiments, initial intron sequences and corresponding intron sequences are paired to facilitate circularization of a nucleic acid molecule. Therefore, in some embodiments, an initial intron sequence may be a paired with any corresponding intron sequence in the right hand column of Table 3, and vice versa.

TABLE 2
Complete Introns

Intron hit 2
AACGTTCAACGACTAATGAGTGAGTTATACCTCAACACGATACCCCCAA
AATCCACTCTATGAGTGGGTTTAAGATATAGTCTAAATATACCCTTAAG
GGTGTAAAGTATAAGTTAAATATTATACGTAATATATTGTGCATTTAGG
AGGTTTTAGTGTAAAATTTGAATTGGACAAGATATTGTTGAGTGCTCCT
TAAAATAGGTAGGGTTATTATTATATTTTAGAGGCAATTGGGGAAAATT
ACAAGGACATTTAATAAATTACCTCCGTTTTATTAGATTATTTGTAGCG
AAACTTAATTCG (SEQ ID NO: 49)
Intron hit 3
CGACATGCATTCCAAGCTTATGAGTTAACGTAAGTCAAGCTCATGTAAA
ATCTGCCTAAAACGGGAAACTCTCACTGAGACAATCCGTTGCTAAATCA
GCTAAATGCCCAACGACTATCCCTGATGAATGTAAGGGAGTAGGGTCAA
GCGACCCGAAACGGCAGACAACTCTAAGAGTTGAAGATATAGTCTGAAC
TGCATGGTGACATGCAGCTGTTTATATAAACGGGTAGAGAAGTAGCGAA
CTCTACTGAACACATTGATGAAGTGAACACGTTATTCAGTTC
(SEQ ID NO: 50)

TABLE 3
Initial and Corresponding Intron Sequences

| Initial Intron | Corresponding Intron |
|---|---|
| Sequence | Sequence |
| Intron hit 2 (A Split) | Intron hit 2 (B Split) |
| Seq | Seq |
| AACGTTCAACGACTAATGAGTGA | AATTTGAATTGGACAAGATATTGTT |
| GTTATACCTCAACACGATACCCC | GAGTGCTCCTTAAAATAGGTAGGGT |
| CAAAATCCACTCTATGAGTGGGT | TATTATTATATTTTAGAGGCAATTG |
| TTAAGATATAGTCTAAATATACC | GGGAAAATTACAAGGACATTTAATA |
| CTTAAGGGTGTAAAGTATAAGTT | AATTACCTCCGTTTTATTAGATTAT |
| AAATATTATACGTAATATATTGT | TTGTAGCGAAACTTAATTCG |
| GCATTTAGGAGGTTTTAGTGTAA | (SEQ ID NO: 46) |
| (SEQ ID NO: 45) | |
| Intron hit 3 (A Split) | Intron hit 3 (B Split) |
| Seq | Seq |
| CGACATGCATTCCAAGCTTATGA | TAAATGCCCAACGACTATCCCTGAT |
| GTTAACGTAAGTCAAGCTCATGT | GAATGTAAGGGAGTAGGGTCAAGCG |
| AAAATCTGCCTAAAACGGGAAAC | ACCCGAAACGGCAGACAACTCTAAG |
| TCTCACTGAGACAATCCGTTGCT | AGTTGAAGATATAGTCTGAACTGCA |
| AAATCAGC | TGGTGACATGCAGCTGTTTATATAA |
| (SEQ ID NO: 47) | ACGGGTAGAGAAGTAGCGAACTCTA |
| | CTGAACACATTGATGAAGTGAACA |
| | CGTTATTCAGTTC (SEQ ID |
| | NO: 48) |

### Protein Coding Sequences

In some embodiments, the nucleic acid molecule comprises a protein or polypeptide coding sequence. In some embodiments, the nucleic acid molecule herein provided includes an IRES that is operably linked to a protein coding sequence. In some embodiments, the nucleic acid molecule includes a protein coding sequence between the IRES sequence and the 3′ intron sequence. In some embodiments, the protein coding sequence is a therapeutic protein or polypeptide. In some embodiments, the protein coding sequence encodes a protein or polypeptide of eukaryotic or prokaryotic origin. In some embodiments, the protein coding sequence encodes a human protein. In some embodiments, the protein coding sequence encodes a non-human protein.

In some embodiments, therapeutic proteins or polypeptides encoded by the nucleic acid molecule disclosed herein have antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity. Some examples of therapeutic proteins or polypeptides may include, but are not limited to, an enzyme replacement protein, a protein for supplementation, a protein vaccination, antigens (e.g. tumor antigens, viral, bacterial), hormones, cytokines, antibodies, immunotherapy (e.g. cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, or a ligand, or a receptor.

In some embodiments, exemplary proteins that can be expressed from the protein coding sequence of the nucleic acid molecules disclosed herein include a receptor binding protein, hormone, growth factor, growth factor receptor modulator, and regenerative protein (e.g., proteins implicated in proliferation and differentiation, e.g., therapeutic protein). In some embodiments, exemplary proteins that can be expressed from the protein coding sequence include enzymes, for instance, oxidoreductase enzymes, metabolic enzymes, mitochondrial enzymes, oxygenases, dehydrogenases, ATP-independent enzyme, and desaturases. In some embodiments, exemplary proteins that can be expressed from the protein coding sequence include an intracellular protein or cytosolic protein. In some embodiments, exemplary polypeptides may be fragments, motifs, active sites, and/or binding sites.

In some embodiments, the protein coding sequence encodes one or more antibodies or fragments thereof. For example, in some embodiments, the protein coding sequence encodes human antibodies of fragments thereof.

The term “antibody” as used herein also includes an “antigen-binding portion” of an antibody (or simply “antibody portion”). The term “antigen-binding portion,” as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16:778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

In some embodiments, the protein coding sequence encodes an intrabody, or an antigen binding fragment thereof. In another embodiment, the intrabody, or antigen binding fragment thereof, is a murine, chimeric, humanized, composite, or human intrabody, or antigen binding fragment thereof. In another embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabody fragments.

In some embodiments, the protein coding sequence encodes a protein for diagnostic use. In some embodiments, the protein coding sequence encodes Gaussia luciferase (Gluc), Firefly luciferase (Fluc), enhanced green fluorescent protein (eGFP), human erythropoietin (hEPO), mScarlet fluorescent protein (e.g., a reporter sequence).

In some embodiments, the protein coding sequence encodes a protein of eukaryotic or prokaryotic origin. In another embodiment, the protein coding sequence encodes human protein or non-human protein. In some embodiments, the protein coding sequence encodes a protein selected from hFIX, SP-B, VEGF-A, human methylmalonyl-CoA mutase (hMUT), CFTR, cancer self-antigens, and additional gene editing enzymes like Cpf1, zinc finger nucleases (ZFNs) or transcription activator-like effector nucleases (TALENs).

In some embodiments, the protein coding sequence encodes a nuclease (e.g., a CRISPR-associated endonuclease and/or a programmable nuclease, such as any nuclease that is capable of gene editing/modification). In some embodiments, the Cas endonuclease is Cas9. The Cas endonuclease may be any Cas endonuclease known in the art. In some embodiments, the Cas protein is Cas9, Cas12 (e.g., Cas12a, Cas12b, Cas12c, Cas12d, or Cas12e), Cas13 (e.g., Cas13a, Cas13b, Cas13c, Cas13d, or Cas13e), Cas 14 (e.g., Cas14a, Cas14b, Cas14c, Cas14d, Cas14e, Cas14f, Cas14g, or Cas14h), CasX, or CasY. In some embodiments, the CRISPR-Cas protein is Cas13b. In some embodiments, the CRISPR-Cas protein is Cas13b-t1, Cas13b-t2, Cas13b-t3. In some embodiments, the CRISPR-Cas is an engineered CRISPR-Cas protein.

In some embodiments, the Cas nuclease is the Cas9 protein from the Type-II CRISPR/Cas system. Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild type Cas9. In some embodiments, the nucleic acid molecule encodes a chimeric Cas nuclease (e.g., a nuclease where one domain or region of the protein is replaced by a portion of a different protein). In some embodiments, a Cas nuclease domain may be replaced with a domain from a different nuclease such as Fok1. In some embodiments, a Cas nuclease may be a modified nuclease.

In some embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the endonuclease is from a type V CRISPR-Cas system. In some embodiments, the endonuclease is from a type VI CRISPR-Cas system. In some embodiments, the programmable nuclease is from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt).

Spacer Sequences In some embodiments, the nucleic acid molecule includes a region of non-coding nucleic acids, such as a spacer sequence. In some embodiments, the spacer sequence is disposed between a 5′ intron sequence and a 3′ sequence, in 5′ to 3′ order. In some embodiments, the nucleic acid molecule includes one or more spacer sequences between the 5′ intron sequence and an IRES sequence, between the IRES sequence and the 3′ intron sequence, between the IRES sequence and a protein coding sequence, and/or between the protein coding sequence and the 3′ intron sequence. In some embodiments, the nucleic acid molecule comprises at least one spacer sequence. In some embodiments, the nucleic acid molecule comprises 1, 2, 3, 4, 5, 6, 7 or more spacer sequences.

In some embodiments, the spacer sequence comprises a sequence of at least 3 nucleotides, at least 4 nucleotides, at least 5 nucleotides, at least about 8 nucleotides, at least about 10 nucleotides, at least about 12 nucleotides, at least about 15 nucleotides, at least about 20 nucleotides, at least about 25 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, at least about 100 nucleotides, at least about 120 nucleotides, at least about 150 nucleotides, at least about 200 nucleotides, at least about 250 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides.

In some embodiments, the spacer sequence may be a nucleic acid sequence or molecule having low GC content, for example less than 65%, 60%, 55%, 50%, 55%, 50%, 45%, 40%, 39%, 38%, 37%, 36%, 35%, 34%, 33%, 32%, 31%, 30%, 29%, 28%, 27%, 26%, 25%, 24%, 23%, 22%, 20%, 19%, 18%, 17%, 16%, 15%, 14%, 13%, 12%, 11%, 10%, 9%, 8%, 7%, 6%, 5%, 4%, 3%, 2% or 1%, across the full length of the spacer, or across at least 50%, 60%, 70%, 80%, 90%, 91%, 92%, 93%, 94%, 95%, 96%, 97%, 98% or 99% contiguous nucleic acid residues of the spacer. In some embodiments, the spacer sequence may comprise at least 95%, 90%, 85%, 80%, 75%, 70%, 65%, 60%, 55%, 50%, 55%, 50%, 45%, 40%, 35%, 30%, 20% or any percentage there between of adenine ribonucleotides. In some embodiments, the spacer sequence comprises at least 5 or more adenine ribonucleotides in a row. In some embodiments, the spacer sequence comprises at least 6 adenine ribonucleotides in a row, at least 7 adenine ribonucleotides in a row, at least 8 ribonucleotides, at least about 10 adenine ribonucleotides in a row, at least about 12 adenine ribonucleotides in a row, at least about 15 adenine ribonucleotides in a row, at least about 20 adenine ribonucleotides in a row, at least about 25 adenine ribonucleotides in a row, at least about 30 adenine ribonucleotides in a row, at least about 40 adenine ribonucleotides in a row, at least about 50 adenine ribonucleotides in a row, at least about 60 adenine ribonucleotides in a row, at least about 70 adenine ribonucleotides in a row, at least about 80 adenine ribonucleotides in a row, at least about 90 adenine ribonucleotides in a row, at least about 95 adenine ribonucleotides in a row, at least about 100 adenine ribonucleotides in a row, at least about 150 adenine ribonucleotides in a row, at least about 200 adenine ribonucleotides in a row, at least about 250 adenine ribonucleotides in a row, at least about 300 adenine ribonucleotides in a row, at least about 350 adenine ribonucleotides in a row, at least about 400 adenine ribonucleotides in a row, at least about 450 adenine ribonucleotides in a row, at least about 500 adenine ribonucleotides in a row, at least about 550 adenine ribonucleotides in a row, or at least about 600 adenine ribonucleotides in a row.

In some embodiments, the spacer sequence is situated between one or more elements (e.g., between a 5′ intron sequence and a 3′ intron sequence). In some embodiments, the spacer sequence provides conformational flexibility between the elements. In some embodiments, the conformational flexibility is due to the spacer sequence being substantially free of a secondary structure. In some embodiments, the spacer sequence is substantially free of a secondary structure, such as less than 40 kcal/mol, less than −39, −38, −37, −36, −35, −34, −33, −32, −31, −30, −29, −28, −27, −26, −25, −24, −23, −22, −20, −19, −18, −17, −16, −15, −14, −13, −12, −11, −10, −9, −8, −7, −6, −5, −4, −3, −2 or −1 kcal/mol. The spacer may include a nucleic acid, such as DNA, RNA, or hybrid DNA-RNA nucleic acids.

As used herein, a “spacer” includes to a region of a polynucleotide sequence ranging from 1 nucleotide to hundreds of nucleotides separating two other elements along a polynucleotide sequence. In some embodiments, the spacer sequence may be non-coding. Where the spacer is a non-coding sequence, a translation initiation sequence may be provided in the coding sequence of an adjacent sequence. In some embodiments, it is envisaged that the first nucleic acid residue of the coding sequence may be the A residue of a translation initiation sequence, such as AUG. A translation initiation sequence may be provided in the spacer sequence. In some embodiments, the spacer is operably linked to another sequence described herein.

### Reporter

In some embodiments, the nucleic acid molecule comprises a reporter sequence. In some embodiments, the reporter sequence is disposed between the 5′ intron sequence and the 3′ intron sequence of the nucleic acid molecule. The particular reporter encoded by the nucleic acid molecule as described herein will vary and depend, in part, upon the preferred method of detection of the produced signal. For example, where the signal is optically detected, e.g., through use of fluorescent microscopy or flow cytometry (including fluorescently activated cell sorting (FACS), a fluorescent reporter may be used.

Suitable detectable signal-producing proteins include, e.g., fluorescent proteins; enzymes that catalyze a reaction that generates a detectable signal as a product; epitope tags, surface markers, and the like. Detectable signal-producing proteins may be directly detected or indirectly detected. For example, where a fluorescent reporter is used, the fluorescence of the reporter may be directly detected. In some instances, where an epitope tag or a surface marker is used, the epitope tag or surface marker may be indirectly detected, e.g., through the use of a detectable binding agent that specifically binds the epitope tag or surface marker, e.g., a fluorescently labeled antibody that specifically binds the epitope tag or surface marker. In some instances, a reporter that is commonly indirectly detected, e.g., an epitope tag or surface marker, may be directly detected or a reporter that is commonly directly detected may be indirectly detected, e.g., through the use of a detectable antibody that specifically binds a fluorescent reporter.

In some embodiments, the reporter sequence encodes a fluorescent protein. In some embodiments, the fluorescent protein is, but is not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

In some embodiments, the reporter encodes an enzyme. In some embodiments, the enzyme is, but is not limited to, horse radish peroxidase (HRP), alkaline phosphatase (AP), β-galactosidase (GAL), glucose-6-phosphate dehydrogenase, β-N-acetylglucosaminidase, β-glucuronidase, invertase, Xanthine Oxidase, firefly luciferase, glucose oxidase (GO), and the like.

In some embodiments, the reporter sequence is a fluorogenic aptamer. Fluorogenic aptamers are well known in the art and include, without limitation, Spinach, Spinach 2, Broccoli, Red-Broccoli, Orange Broccoli, Corn, Mango, Malachite Green, cobalamine-binding aptamer, and derivatives thereof. See, e.g., Autour et al., “Fluorogenic RNA Mango Aptamers for Imaging Small Non-Coding RNAs in Mammalian Cells,” Nature Comm. 9: Article 656 (2018); Jaffrey, S., “RNA-Based Fluorescent Biosensors for Detecting Metabolites In Vitro and in Living Cells,” Adv Pharmacol. 82:187-203 (2018); and Litke et al., “Developing Fluorogenic Riboswitches for Imaging Metabolite Concentration Dynamics in Bacterial Cells,” Methods Enzymol. 572:315-33 (2016), each of which are hereby incorporated by reference in their entirety). In accordance with this embodiment, the fluorogenic aptamer binds to a fluorophore whose fluorescence, absorbance, spectral properties, or quenching properties are increased, decreased, or altered by interaction with the fluorogenic aptamer. Any aptamer-dye complex, some of which are fluorogenic aptamers, may be used. In addition, some aptamers can bind quenchers and some do other things to change the photophysical properties of dyes. In another embodiment, the aptamer binds a target molecule of interest. The target molecule of interest may be any biomaterial or small molecule including, without limitation, proteins, nucleic acids (RNA or DNA), lipids, oligosaccharides, carbohydrates, small molecules, hormones, cytokines, chemokines, cell signaling molecules, metabolites, organic molecules, and metal ions. The target molecule of interest may be one that is associated with a disease state or pathogen infection.

In some embodiments, the reporter sequence includes a fluorogenic aptamer coupled to an aptamer that binds a target molecule. In some embodiments, the reporter sequence of interest is a sensor. In some embodiments, the fluorogenic aptamer is coupled to an aptamer that binds a target molecule using a transducer stem. Suitable target molecules of interest include, but are not limited to, ADP, adenosine, guanine, GTP, SAM, and streptavidin.

### Translation Regulation Motif

In some aspects, provided herein are nucleic acid molecules that comprise a translation regulation motif. Translation regulation motifs include, but are not limited to, RNA sequences and/or structures that are commonly located in the untranslated regions of RNA transcripts (e.g., 5′ and/or 3′ to a protein coding sequence) and can also include the initiation codon. Translation regulation motifs may be recognized by regulatory proteins or micro RNAs (miRNAs).

A translation regulation motif may include a sequence that is located adjacent to an expression sequence that encodes an expression product. A translation regulation motif may be linked operatively to the adjacent sequence. A translation regulation motif may increase an amount of product expressed as compared to an amount of the expressed product when no translation regulation motif exists. In addition, one translation regulation motif can increase a number of products expressed for multiple expression sequences attached in tandem. Hence, one translation regulation motif can enhance the expression of one or more expression sequences. Multiple translation regulation motifs are well-known to persons of ordinary skill in the art.

A translation regulation motif as provided herein can be a nucleic acid sequence that selectively initiates or activates translation of an expression sequence in the nucleic acid molecule, for instance, certain riboswitch aptazymes. A translation regulation motif can also include a selective degradation sequence. As used herein, the term “selective degradation sequence” can refer to a nucleic acid sequence that initiates degradation of the nucleic acid molecule, or an expression product of the nucleic acid molecule. Exemplary selective degradation sequence can include riboswitch aptazymes and miRNA binding sites.

In some embodiments, the translation regulation motif is a translation modulator. A translation modulator can modulate translation of the expression sequence in the nucleic acid molecule. A translation modulator can be a translation enhancer or suppressor. In some embodiments, the nucleic acid molecule includes at least one translation modulator adjacent to at least one expression sequence. In some embodiments, the nucleic acid molecule includes a translation modulator adjacent each expression sequence. In some embodiments, the translation modulator is present on one or both sides of each expression sequence, leading to separation of the expression products, e.g., peptide(s) and or polypeptide(s).

In some embodiments, a translation initiation sequence can function as a translation regulation motif. In some embodiments, a translation initiation sequence comprises an AUG codon. In some embodiments, a translation initiation sequence comprises any eukaryotic start codon such as AUG, CUG, GUG, UUG, ACG, AUC, AUU, AAG, AUA, or AGG. In some embodiments, a translation initiation sequence comprises a Kozak sequence. In some embodiments, translation begins at an alternative translation initiation sequence, e.g., translation initiation sequence other than AUG codon, under selective conditions, e.g., stress induced conditions. As a non-limiting example, the translation of the nucleic acid molecule may begin at an alternative translation initiation sequence, such as ACG. As another non-limiting example, the nucleic acid molecule translation may begin at an alternative translation initiation sequence, CTG/CUG. As yet another non-limiting example, the nucleic acid molecule translation may begin at an alternative translation initiation sequence, GTG/GUG. As yet another non-limiting example, the nucleic acid molecule may begin translation at a repeat-associated non-AUG (RAN) sequence, such as an alternative translation initiation sequence that includes short stretches of repetitive RNA e.g., CGG, GGGGCC, CAG, CTG.

Nucleotides flanking a codon that initiates translation, such as, but not limited to, a start codon or an alternative start codon, are known to affect the translation efficiency, the length and/or the structure of the nucleic acid molecule. (See e.g., Matsuda and Mauro PLOS ONE, 2010 5:11; the contents of which are herein incorporated by reference in its entirety). Masking any of the nucleotides flanking a codon that initiates translation may be used to alter the position of translation initiation, translation efficiency, length and/or structure of the nucleic acid molecule.

In one embodiment, a masking agent may be used near the start codon or alternative start codon in order to mask or hide the codon to reduce the probability of translation initiation at the masked start codon or alternative start codon. Non-limiting examples of masking agents include antisense locked nucleic acids (LNA) oligonucleotides and exon junction complexes (EJCs). (See e.g., Matsuda and Mauro describing masking agents LNA oligonucleotides and EJCs (PLOS ONE, 2010 5:11); the contents of which are herein incorporated by reference in its entirety). In another embodiment, a masking agent may be used to mask a start codon of the nucleic acid molecule in order to increase the likelihood that translation will initiate at an alternative start codon.

In some embodiments, the nucleic acid molecule encodes a protein and may comprise a translation initiation sequence, e.g., a start codon. In some embodiments, the translation initiation sequence includes a Kozak or Shine-Dalgarno sequence. In some embodiments, the nucleic acid molecule includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the nucleic acid molecule includes at least one translation initiation sequence adjacent to an expression sequence. In some embodiments, the translation initiation sequence provides conformational flexibility to the nucleic acid molecule. In some embodiments, the translation initiation sequence is within a substantially single stranded region of the nucleic acid molecule.

In some embodiments, the nucleic acid molecule may include more than 1 start codon such as, but not limited to, at least 2, at least 3, at least 4, at least 5, at least 6, at least 7, at least 8, at least 9, at least 10 start codons. Translation may initiate on the first start codon or may initiate downstream of the first start codon.

In some embodiments, the nucleic acid molecule may initiate at a codon which is not the first start codon, e.g., AUG. Translation of the nucleic acid molecule may initiate at an alternative translation initiation sequence, such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLOS ONE, 2010 5:11; the contents of each of which are herein incorporated by reference in their entireties). In some embodiments, translation begins at an alternative translation initiation sequence under selective conditions, e.g., stress induced conditions. As a non-limiting example, the translation of the nucleic acid molecule may begin at alternative translation initiation sequence, such as ACG. As another non-limiting example, the nucleic acid molecule translation may begin at alternative translation initiation sequence, CTG/CUG. As yet another non-limiting example, the nucleic acid molecule translation may begin at alternative translation initiation sequence, GTG/GUG. As yet another non-limiting example, the nucleic acid molecule may begin translation at a repeat-associated non-AUG (RAN) sequence, such as an alternative translation initiation sequence that includes short stretches of repetitive RNA e.g., CGG, GGGGCC, CAG, CTG.

In some embodiments, the nucleic acid molecule includes a termination element (e.g., a translational terminator). In some embodiments, a termination element is capable of terminating translation of a protein coding sequence. In some embodiments, a termination element is situated 3′ to a protein coding sequence or between the protein coding sequence and the 3′ intron sequence. In some embodiments, a nucleic acid can comprise multiple optional translation regulation motifs (e.g., a translation enhancer, a translational initiation sequence, and/or a termination element, etc, etc, etc.).

In some embodiments, a nucleic acid comprises, in order from 5′ to 3′, a 5′ intron sequence, an IRES sequence, a protein coding sequence, a 3′ intron sequence, wherein any number of optional translation regulation motifs and/or spacers can be situated between the 5′ intron sequence and the IRES sequence, between the IRES sequence and the protein coding sequence, and/or between the protein coding sequence and the 3′ intron sequence. In some embodiments, the nucleic acid molecule includes one or more protein coding sequences and the protein coding sequences lack a termination element, such that the nucleic acid molecule is continuously translated. Exclusion of a termination element may result in rolling circle translation or continuous expression of expression product, e.g., proteins, peptides, or polypeptides, due to lack of ribosome stalling or fall-off. In such an embodiment, rolling circle translation expresses a continuous expression product through each protein coding sequence. In some other embodiments, a termination element of a protein coding sequence can be part of a stagger element. In some embodiments, one or more protein coding sequences in the nucleic acid molecule comprises a termination element. However, rolling circle translation or expression of a succeeding (e.g., second, third, fourth, fifth, etc.) protein coding sequence in the nucleic acid molecule is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the nucleic acid molecule.

In some embodiments, the nucleic acid molecule includes a termination element at the end of one or more protein coding sequences. In some embodiments, one or more protein coding sequences comprises two or more termination elements in succession. In such embodiments, translation is terminated and rolling circle translation is terminated. In some embodiments, the ribosome completely disengages from the nucleic acid molecule. In some such embodiments, expression of a succeeding (e.g., second, third, fourth, fifth, etc.) protein coding sequence in the nucleic acid molecule may require the ribosome to reengage with the nucleic acid molecule prior to initiation of translation. Generally, termination elements include an in-frame nucleotide triplet that signals termination of translation, e.g., UAA, UGA, UAG. In some embodiments, one or more termination elements in the nucleic acid molecule are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of a protein coding sequence. Frame-shifted termination elements may be important in preventing misreads of mRNA, which is often detrimental to the cell.

In some embodiments, the nucleic acid molecule produces stoichiometric ratios of expression products. Rolling circle translation continuously produces expression products at substantially equivalent ratios. In some embodiments, the nucleic acid molecule has a stoichiometric translation efficiency, such that expression products are produced at substantially equivalent ratios. In some embodiments, the nucleic acid molecule has a stoichiometric translation efficiency of multiple expression products, e.g., products from 2, 3, 4, 5, 6, 7, 8, 9, 10, 11, 12, or more expression sequences.

In some embodiments, once translation of the nucleic acid molecule is initiated, the ribosome bound to the nucleic acid molecule does not disengage from the nucleic acid molecule before finishing at least one round of translation of the nucleic acid molecule. In some embodiments, the nucleic acid molecule as described herein is competent for rolling circle translation. In some embodiments, during rolling circle translation, once translation of the nucleic acid molecule is initiated, the ribosome bound to the nucleic acid molecule does not disengage from the nucleic acid molecule before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 105 rounds, or at least 106 rounds of translation of the nucleic acid molecule.

In some embodiments, the rolling circle translation of the nucleic acid molecule leads to generation of polypeptide product that is translated from more than one round of translation of the nucleic acid molecule (“continuous” expression product). In some embodiments, the nucleic acid molecule comprises a stagger element, and rolling circle translation of the nucleic acid molecule leads to generation of polypeptide product that is generated from a single round of translation or less than a single round of translation of the nucleic acid molecule (“discrete” expression product). In some embodiments, the nucleic acid molecule is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the nucleic acid molecule are discrete polypeptides.

### Modified Bases

The nucleic acid molecules disclosed herein may have one or more modified bases. In some embodiments, a “modified base” is a ribonucleotide base of uracil, cytosine, adenine, or guanine that possesses a chemical modification from its normal structure. For example, one type of modified base is a methylated base, such as N6-methyladenosine (m6A). A modified base may also be a substituted base, meaning the base possesses a structural modification that renders it a chemical entity other than uracil, cytosine, adenine, or guanine. For example, pseudouridine is one type of substituted RNA base. Table 4 below provides a list of exemplary modified bases that may be present in a nucleic acid molecule described herein.

TABLE 4
List of Exemplary Base Modifications

| Abbreviation | Chemical name |
|---|---|
| m¹acp³Y | 1-methyl-3-(3-amino-3-carboxypropyl) pseudouridine |
| m¹A | 1-methyladenosine |
| m¹G | 1-methylguanosine |
| m¹I | 1-methylinosine |
| m¹Y | 1-methylpseudouridine |
| m¹Am | 1,2′-O-dimethyladenosine |
| m¹Gm | 1,2′-O-dimethylguanosine |
| m¹Im | 1,2′-O-dimethylinosine |
| m²A | 2-methyladenosine |
| ms²io⁶A | 2-methylthio-N⁶-(cis-hydroxyisopentenyl) adenosine |
| ms²hn⁶A | 2-methylthio-N⁶-hydroxynorvalyl carbamoyladenosine |
| ms²i⁶A | 2-methylthio-N⁶-isopentenyladenosine |
| ms²m⁶A | 2-methylthio-N⁶-methyladenosine |
| ms²t⁶A | 2-methylthio-N⁶-threonyl carbamoyladenosine |
| s²Um | 2-thio-2′-O-methyluridine |
| s²C | 2-thiocytidine |
| s²U | 2-thiouridine |
| Am | 2′-O-methyladenosine |
| Cm | 2′-O-methylcytidine |
| Gm | 2′-O-methylguanosine |
| Im | 2′-O-methylinosine |
| Ym | 2′-O-methylpseudouridine |
| Um | 2′-O-methyluridine |
| Ar(P) | 2′-O-ribosyladenosine (phosphate) |
| Gr(p) | 2′-O-ribosylguanosine (phosphate) |
| acp³U | 3-(3-amino-3-carboxypropyl)uridine |
| m³C | 3-methylcytidine |
| m³Y | 3-methylpseudouridine |
| m³U | 3-methyluridine |
| m³Um | 3,2′-O-dimethyluridine |
| imG-14 | 4-demethylwyosine |
| s⁴U | 4-thiouridine |
| chm⁵U | 5-(carboxyhydroxymethyl)uridine |
| mchm⁵U | 5-(carboxyhydroxymethyl)uridine methyl ester |
| inm⁵s²U | 5-(isopentenylaminomethyl)-2-thiouridine |
| inm⁵Um | 5-(isopentenylaminomethyl)-2′-O-methyluridine |
| inm⁵U | 5-(isopentenylaminomethyl)uridine |
| nm⁵s²U | 5-aminomethyl-2-thiouridine |
| ncm⁵Um | 5-carbamoylmethyl-2′-O-methyluridine |
| ncm⁵U | 5-carbamoylmethyluridine |
| cmnm⁵Um | 5-carboxymethylaminomethyl-2′-O-methyluridine |
| cmnm⁵s²U | 5-carboxymethylaminomethyl-2-thiouridine |
| cmnm⁵U | 5-carboxymethylaminomethyluridine |
| cm⁵U | 5-carboxymethyluridine |
| f⁵Cm | 5-formyl-2′-O-methylcytidine |
| f⁵C | 5-formylcytidine |
| hm⁵C | 5-hydroxymethylcytidine |
| ho⁵U | 5-hydroxyuridine |
| mcm⁵s²U | 5-methoxycarbonylmethyl-2-thiouridine |
| mcm⁵Um | 5-methoxycarbonylmethyl-2′-O-methyluridine |
| mcm⁵U | 5-methoxycarbonylmethyluridine |
| mo⁵U | 5-methoxyuridine |
| m⁵s²U | 5-methyl-2-thiouridine |
| mnm⁵se²U | 5-methylaminomethyl-2-selenouridine |
| mnm⁵s²U | 5-methylaminomethyl-2-thiouridine |
| mnm⁵U | 5-methylaminomethyluridine |
| m⁵C | 5-methylcytidine |
| m⁵D | 5-methyldihydrouridine |
| m⁵U | 5-methyluridine |
| tm⁵s²U | 5-taurinomethyl-2-thiouridine |
| tm⁵U | 5-taurinomethyluridine |
| m⁵Cm | 5,2′-O-dimethylcytidine |
| m⁵Um | 5,2′-O-dimethyluridine |
| preQ₁ | 7-aminomethyl-7-deazaguanosine |
| preQ₀ | 7-cyano-7-deazaguanosine |
| m⁷G | 7-methylguanosine |
| G⁺ | archaeosine |
| D | dihydrouridine |
| oQ | epoxyqueuosine |
| galQ | galactosyl-queuosine |
| OHyW | hydroxywybutosine |
| I | inosine |
| imG2 | isowyosine |
| k²C | lysidine |
| manQ | mannosyl-queuosine |
| mimG | methylwyosine |
| m²G | N²-methylguanosine |
| m²Gm | N²,2′-O-dimethylguanosine |
| m^{2,7}G | N²,7-dimethylguanosine |
| m^{2,7}Gm | N²,7,2′-O-trimethylguanosine |
| m² ₂G | N²,N²-dimethylguanosine |
| m² ₂Gm | N²,N²,2′-O-trimethylguanosine |
| m^{2,2,7}G | N²,N²,7-trimethylguanosine |
| ac⁴Cm | N⁴-acetyl-2′-O-methylcytidine |
| ac⁴C | N⁴-acetylcytidine |
| m⁴C | N⁴-methylcytidine |
| m⁴Cm | N⁴,2′-O-dimethylcytidine |
| m⁴ ₂Cm | N⁴,N⁴,2′-O-trimethylcytidine |
| io⁶A | N⁶-(cis-hydroxyisopentenyl)adenosine |
| ac⁶A | N⁶-acetyladenosine |
| g⁶A | N⁶-glycinylcarbamoyladenosine |
| hn⁶A | N⁶-hydroxynorvalylcarbamoyladenosine |
| i⁶A | N⁶-isopentenyladenosine |
| m⁶t⁶A | N⁶-methyl-N⁶-threonylcarbamoyladenosine |
| m⁶A | N⁶-methyladenosine |
| t⁶A | N⁶-threonylcarbamoyladenosine |
| m⁶Am | N⁶,2′-O-dimethyladenosine |
| m⁶ ₂A | N⁶,N⁶,-dimethyladenosine |
| m⁶ ₂Am | N⁶,N⁶,2′-O-trimethyladenosine |
| o₂yW | peroxywybutosine |
| Y | pseudouridine |
| Q | queuosine |
| OHyW | undermodified hydroxywy butosine |
| cmo⁵U | uridine 5-oxyacetic acid |
| mcmo⁵U | uridine 5-oxyacetic acid methyl ester |
| yW | wybutosine |
| imG | wyosine |

The nucleic acid molecule may include one or more substitutions, insertions and/or additions, deletions, and covalent modifications. In some embodiments, the nucleic acid molecule includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27:196-197). In some embodiments, the nucleic acid molecule comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleic acid molecule includes any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof).

The nucleic acid molecules can be comprised wholly of naturally occurring nucleic acids, or in certain aspects can contain one or more nucleic acid analogues or derivatives. The nucleic acid analogues can include backbone analogues and/or nucleic acid base analogues and/or utilize non-naturally occurring base pairs. Illustrative artificial nucleic acids that can be used in the present constructs include, without limitation, nucleic backbone analogs peptide nucleic acids (PNA), morpholino and locked nucleic acids (LNA), bridged nucleic acids (BNA), glycol nucleic acids (GNA) and threose nucleic acids (TNA). Nucleic acid base analogues that can be used in the present constructs include, without limitation, fluorescent analogs (e.g., 2-aminopurine (2-AP), 3-Methylindole (3-MI), 6-methyl isoxanthoptherin (6-MI), 6-MAP, pyrrolo-dC and derivatives thereof, furan-modified bases, 1,3-Diaza-2-oxophenothiazine (tC), 1,3-diaza-2-oxophenoxazine); non-canonical bases (e.g., inosine, thiouridine, pseudouridine, dihydrouridine, queuosine and wyosine), 2-aminoadenine, thymine analogue 2,4-difluorotoluene (F), adenine analogue 4-methylbenzimidazole (Z), isoguanine, isocytosine; diaminopyrimidine, xanthine, isoquinoline, pyrrolo[2,3-b]pyridine; 2-amino-6-(2-thienyl) purine, pyrrole-2-carbaldehyde, and universal bases (e.g., 2′ deoxyinosine (hypoxanthine deoxynucleotide) derivatives, nitroazole analogues). Non-naturally occurring base pairs that can be used in the present nucleic acid molecules include, without limitation, isoguanine and isocytosine; diaminopyrimidine and xanthine; 2-aminoadenine and thymine; isoquinoline and pyrrolo[2,3-b]pyridine; 2-amino-6-(2-thienyl) purine and pyrrole-2-carbaldehyde; two 2,6-bis(ethylthiomethyl)pyridine (SPy) with a silver ion; pyridine-2,6-dicarboxamide (Dipam) and a mondentate pyridine (Py) with a copper ion.

In some embodiments, the nucleic acid molecule includes at least one N(6)methyladenosine (m6A) modification to increase translation efficiency. In some embodiments, the N(6)methyladenosine (m6A) modification can reduce immunogeneicity of the nucleic acid molecule. In some embodiments, the modification may include a chemical or cellular induced modification. For example, some non-limiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the ribonucleotides of the nucleic acid molecule may enhance immune evasion. The nucleic acid molecule may be synthesized and/or modified by methods well established in the art, such as those described in “Current protocols in nucleic acid chemistry,” Beaucage, S. L. et al. (Eds.), John Wiley & Sons, Inc., New York, N.Y., USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. In some embodiments, the bases of the modified nucleic acid molecule include 5-methylcytidine and/or pseudouridine. In some embodiments, base modifications may modulate expression, immune response, stability, subcellular localization, to name a few functional effects, of the nucleic acid molecule. In some embodiments, the modification includes a bi-orthogonal nucleotides, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more nucleotides of the nucleic acid molecule may, as well as backbone modifications, include modification or replacement of the phosphodiester linkages. Specific examples of nucleic acid molecule include, but are not limited to nucleic acid molecule including modified backbones or no natural internucleoside linkages such as internucleoside modifications, including modification or replacement of the phosphodiester linkages. Nucleic acid molecules having modified backbones include, among others, those that do not have a phosphorus atom in the backbone. In particular embodiments, the nucleic acid molecule will include ribonucleotides with a phosphorus atom in its internucleoside backbone.

In some embodiments, modified nucleic acid molecule backbones include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the nucleic acid molecule may be negatively or positively charged.

The modified nucleotides, which may be incorporated into the nucleic acid molecule, can be modified on the internucleoside linkage (e.g., phosphate backbone). Herein, in the context of the polynucleotide backbone, the phrases “phosphate” and “phosphodiester” are used interchangeably. In some embodiments, backbone phosphate groups are modified by replacing one or more of the oxygen atoms with a different substituent. In some embodiments, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. In some embodiments, the phosphate linker can also be modified by the replacement of a linking oxygen with nitrogen (bridged phosphoramidates), sulfur (bridged phosphorothioates), and carbon (bridged methylene-phosphonates).

In some embodiments, a thio substituted phosphate moiety is provided to confer stability to RNA and DNA polymers through the unnatural phosphorothioate backbone linkages. In some embodiments, phosphorothioate DNA and RNA have increased nuclease resistance and subsequently a longer half-life in a cellular environment. In some embodiments, phosphorothioate linked to the nucleic acid molecule is expected to reduce the innate immune response through weaker binding/activation of cellular innate immune molecules.

In some embodiments, a modified nucleoside includes an alpha-thio-nucleoside (e.g., 5′-0-(1-thiophosphate)-adenosine, 5′-0-(1-thiophosphate)-cytidine (a-thio-cytidine), 5′-0-(1-thiophosphate)-guanosine, 5′-0-(1-thiophosphate)-uridine, or 5′-0-(1-thiophosphate)-pseudouridine).

In some embodiments, the nucleic acid molecule may include one or more cytotoxic nucleosides. For example, cytotoxic nucleosides may be incorporated into nucleic acid molecule, such as bifunctional modification. In some embodiments, cytotoxic nucleosides include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4 (1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the nucleic acid molecule may or may not be uniformly modified along the entire length of the molecule. In some embodiments, one or more or all types of nucleotides (e.g., naturally occurring nucleotides, purine or pyrimidine, or any one or more or all of A, G, U, C, I, pU) are, or are not, uniformly modified in the nucleic acid molecule, or in a given predetermined sequence region thereof. In some embodiments, the nucleic acid molecule includes a pseudouridine. In some embodiments, the nucleic acid molecule includes an inosine, which may aid in the immune system characterizing the nucleic acid molecule as endogenous versus viral RNAs. The incorporation of inosine may also mediate improved RNA stability/reduced degradation. See for example, Yu, Z. et al. (2015) RNA editing by ADARI marks dsRNA as “self”. Cell Res. 25, 1283-1284, which is incorporated by reference in its entirety.

In some embodiments, a modification is in a non-coding region of the nucleic acid molecule provided herein. In some embodiments, the nucleic acid molecule includes from about 1% to about 100% modified nucleotides (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, or from 95% to 100%).

### Constructs

In certain aspects, the disclosure provides a nucleic acid construct comprising any of the herein described nucleic acid molecules. In some embodiments, the construct is a linear primary construct that is circularized or undergoes concatemerization through methods such as, but not limited to, chemical, enzymatic, splint ligation, or ribozyme catalyzed methods. In some embodiments, the construct is a circular primary construct.

In some embodiments, provided herein are DNA plasmids and viral replicating vectors comprising nucleic acid molecules as described above and herein. In some embodiments, the entire size of the DNA plasmids designed are from about 2000 bp to about 15,000 bp (e.g., about 5,000 bp, about 6,000 bp, about 7,000 bp, about 8,000 bp, about 9,000 bp, about 10,000 bp, about 12,000 bp, about 14,000 bp, about 15,000 bp, about 16,000 bp, about 17,000 bp, about 18,000 bp, about 19,000 bp, or about 20,000 bp). Generally, the plasmid backbone comprises an origin of replication and an expression cassette for expressing a sequence of interest and/or a selection gene. In some embodiments, the expression cassette for expressing a selection gene is in the antisense orientation from the central ribozyme. The selection gene can be any marker known in the art for selection of a host cell that has been transformed with a desired plasmid. In some embodiments, the selection marker comprises a polynucleotide encoding a gene or protein conferring antibiotic resistance, heat tolerance, fluorescence, or luminescence.

In some embodiments, viral replicating vectors can be used to express the DNA or RNA constructs as described. In planta, gemini viruses are a representative DNA virus that can be used as an expression system (reviewed in, e.g., Hefferon, Vaccines (2014) 2:642-53). In animal cells, there are more choices. In some embodiments, plasmid expression constructs containing viral origins of replication, while not truly viral replicating systems, are stably maintained in cells. In some embodiments, truly replicating viral systems of use include, without limitation, adenovirus, adeno-associated virus, baculovirus, and Vaccinia virus vectors, which are known in the art.

In some aspects, the one or more DNA constructs, as described above and herein, are first transcribed in vitro into RNA and then the RNA transcript is transfected into a host cell. The step of transcribing the one or more DNA constructs into RNA in vitro can be performed using any methodologies known in the art. In vitro transcription of one or more (e.g., a population of) DNA constructs comprising a library of inserts containing a nucleic acid sequence of interest can be achieved using purified RNA polymerases, e.g., T7 RNA polymerase. Such methodologies are described in, e.g., Green and Sambrook, Molecular Cloning, A Laboratory Manual, 4th Ed., Cold Spring Harbor Press, (2012).

### Methods of Generating and Delivering Nucleic Acid Molecules

In certain aspects, the disclosure provides methods of generating the nucleic acid molecules herein described. In some embodiments, the methods include expressing the nucleic acid molecules (e.g., circular and/or linear) or the constructs acids as herein described.

In some embodiments, the circular nucleic acid molecule is produced using recombinant technology (methods described in detail below; e.g., derived in vitro using a DNA plasmid) or chemical synthesis.

The nucleic acid molecules may be prepared according to any available technique including, but not limited to chemical synthesis and enzymatic synthesis. In some embodiments, a linear primary construct or linear mRNA may be cyclized, or concatemerized to create a nucleic acid molecule described herein as previously described. The mechanism of cyclization or concatemerization may occur through methods such as, but not limited to, chemical, enzymatic, splint ligation), or ribozyme catalyzed methods. The newly formed 5′-/3′-linkage may be an intramolecular linkage or an intermolecular linkage.

In some embodiments, a ribozyme ligase reaction takes from about 1 hour to 24 hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours). In some embodiments, a ribozyme ligase reaction is performed at temperatures between about 0° C. and about 37° C. (e.g., about 0° C., about 1° C., about 1.5° C., about 2° C., about 2.5° C., about 3° C., about 3.5° C., about 4° C., about 4.5° C., about 5° C., about 5.5° C., about 6° C., about 6.5° C., about 7° C., about 7.5° C., about 8° C., about 8.5° C., about 9° C., about 9.5° C., about 10° C., about 10.5° C., about 11° C., about 11.5° C., about 12° C., about 12.5° C., about 13° C., about 13.5° C., about 14° C., about 14.5° C., about 15° C., about 15.5° C., about 16° C., about 16.5° C., about 17° C., about 17.5° C., about 18° C., about 18.5° C., about 19° C., about 19.5° C., about 20° C., about 20.5° C., about 21° C., about 21.5° C., about 22° C., about 22.5° C., about 23° C., about 23.5° C., about 24° C., about 24.5° C., about 25° C., about 25.5° C., about 26° C., about 26.5° C., about 27° C., about 27.5° C., about 28° C., about 28.5° C., about 29° C., about 29.5° C., about 30° C., about 30.5° C., about 31° C., about 31.5° C., about 32° C., about 32.5° C., about 33° C., about 33.5° C., about 34° C., about 34.5° C., about 35° C., about 35.5° C., about 36° C., about 36.5° C., or about 37° C.).

Methods of making the nucleic acid described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012). Various methods of synthesizing circular polyribonucleotides are also described in the art (see, e.g., U.S. Pat. Nos. 6,210,931, 5,773,244, 5,766,903, 5,712,128, 5,426,180, US Publication No. US20100137407, International Publication No. WO1992001813 and International Publication No. WO2010084371; the contents of each of which are herein incorporated by reference in their entireties).

In certain embodiments, the step of isolating the nucleic acid molecules is performed using any appropriate methodology known in the art. Examples of such methodologies are described in, e.g., Green and Sambrook, Molecular Cloning, A Laboratory Manual, 4th Ed., Cold Spring Harbor Press, (2012). For example, provided herein are methods of purifying nucleic acid molecules, comprising running the nucleic acid molecule through a size-exclusion column in tris-EDTA or citrate buffer in a high-performance liquid chromatography (HPLC) system. In another embodiment, the nucleic acid molecule is run through the size-exclusion column in tris-EDTA or citrate buffer at pH in the range of about 4-7 at a flow rate of about 0.01-5 mL/minute. In one embodiment, the HPLC removes one or more of: intron fragments, nicked linear RNA, linear and circular concatenations, and impurities resulting from in vitro transcription and splicing reactions.

In certain aspects, provided herein are methods of making circular RNA, said method comprising using a nucleic acid molecule provided herein. In some embodiments, the method comprises a.) synthesizing RNA by in vitro transcription of a nucleic acid molecule, and b.) incubating the RNA in the presence of magnesium ions and guanosine nucleotide or nucleoside at a temperature at which RNA circularization occurs (e.g., between 20° C. and 60° C.).

In some embodiments, provided herein is a method of expressing protein in a cell, said method comprising transfecting the nucleic acid molecule into the cell. In some embodiments, the method includes transfecting using lipofection or electroporation. In some embodiments, the nucleic acid molecule is transfected into a cell using a nanocarrier. In some embodiments, the nanocarrier is a lipid, polymer or a lipo-polymeric hybrid.

In some embodiments, the DNA construct or in vitro transcribed RNA construct is transfected into a suitable host cell of closed circular DNA plasmid using any method known in the art, e.g., by electroporation of protoplasts, fusion of liposomes to cell membranes, cell transfection methods using calcium ions or PEG, use of gold or tungsten microparticles coated with plasmid with the gene gun. Such methodologies are described in, e.g., Green and Sambrook, Molecular Cloning, A Laboratory Manual, 4th Ed., Cold Spring Harbor Press, (2012). In some embodiments, cells of eukaryotic organisms (plants, animals, fungi, etc.) can be used. In some aspects, the host cell is a prokaryotic cell, e.g., a bacterial cell, an archaeal cell, or an archaebacterial cell.

In certain embodiments, for in vivo transcription of a full-length nucleic acid molecule, the nucleic acid molecule comprises a binding site that is active and induces transcription in a host cell that comprises the nucleic acid molecule. For example, if a DNA construct is introduced into a eukaryotic cell, a selected 5′ or upstream binding site is biologically active for generating RNA in the eukaryotic cell. As appropriate, the 5′ or upstream binding site can be a mammalian promoter that actively promotes transcription in a mammalian host cell. In some embodiments, the 5′ or upstream binding site can be a plant binding site that actively promotes transcription in a plant host cell.

In embodiments of the present disclosure, the nucleic acid molecule products described herein and/or produced using the nucleic acid molecules and/or methods described herein, may be provided in compositions, e.g., pharmaceutical compositions.

In some embodiments, provided herein are compositions, e.g., compositions comprising a nucleic acid molecule and a pharmaceutically acceptable carrier. In one aspect, the present disclosure provides pharmaceutical compositions comprising an effective amount of a nucleic acid molecule described herein and a pharmaceutically acceptable excipient. Pharmaceutical compositions of the present disclosure may comprise a RNA and or DNA molecule as described herein, in combination with one or more pharmaceutically or physiologically acceptable carriers, excipients or diluents. In some embodiments, pharmaceutical compositions of the present disclosure may comprise a nucleic acid molecule expressing cell, e.g., a plurality of r nucleic acid molecule-expressing cells, as described herein, in combination with one or more pharmaceutically or physiologically acceptable carriers, excipients or diluents.

In some embodiments, a pharmaceutically acceptable carrier can be an ingredient in a pharmaceutical composition, other than an active ingredient, which is nontoxic to the subject. A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, saccharides, antioxidants, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

In some embodiments, such compositions may comprise buffers such as acetic acid, citric acid, histidine, boric acid, formic acid, succinic acid, phosphoric acid, carbonic acid, malic acid, aspartic acid, Tris buffers, HEPPSO, HEPES, neutral buffered saline, phosphate buffered saline and the like; carbohydrates such as glucose, sucrose, mannose, or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

In certain embodiments, compositions of the present disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which may be buffered to a desirable pH.

### Examples

The following examples are provided to further illustrate some embodiments of the present disclosure, but are not intended to limit its scope; it will be understood by their exemplary nature that other procedures, methodologies, or techniques known to those skilled in the art may alternatively be used.

A platform for custom design, optimization, and manufacturing of circRNAs was developed. The platform relies on sequence mining and construct design engineering to improve the generation of circRNAs and their ability to support protein expression over an extended period of time. Additionally, diverse mechanisms of circularization for more efficient production of circular RNAs have been generated. These designs are modifiable to express a payload in various cell lines, and in some cases, in a cell-type specific manner.

Using the platform developed to engineer and/or screen diverse classes of circular RNAs, several synthetic IRESs were designed and identified, and several non-synthetic IRES's were identified, as good translator-drivers of reporter proteins like Green Fluorescent Protein (GFP). A skilled artisan will recognize that these IRESs may also be used as translator-drivers of other proteins or polypeptides.

As shown herein, Applicant also developed intron sequences for circularization of nucleic acid molecules. Novel Group I introns were used to self-splice linear RNAs into circular RNAs. Group I introns are selfish genetic elements found in the transcripts of many domains of life, including bacteria, archaea, fungi and viruses. These introns possess ribozymatic activity which allows them to self-splice from their host transcripts. RNAs which contain split group I introns, permuted so that the donor site is 3′ of the acceptor site, are self-circularizing, and such constructs can be used to produce protein encoding, self-circularizing RNAs.

### Example 1—DNA Template Generation

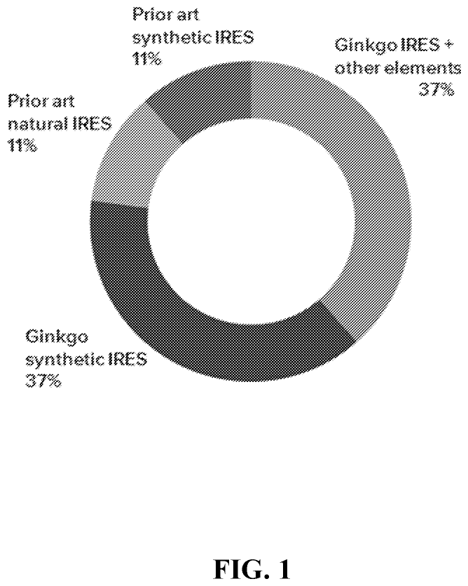

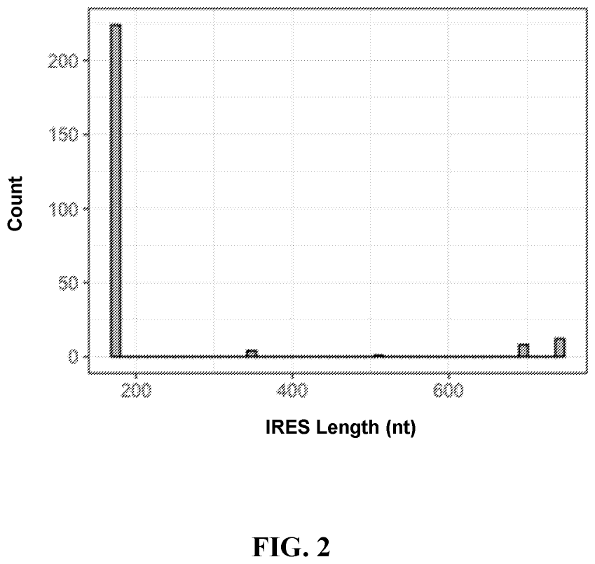

Plasmid DNA libraries containing a diverse group of IRESs were designed (FIG. 1). The library was composed of IRES sequences from various sources (FIG. 1) and of various size ranges (FIG. 2) with most constructs at 174 nt in length.

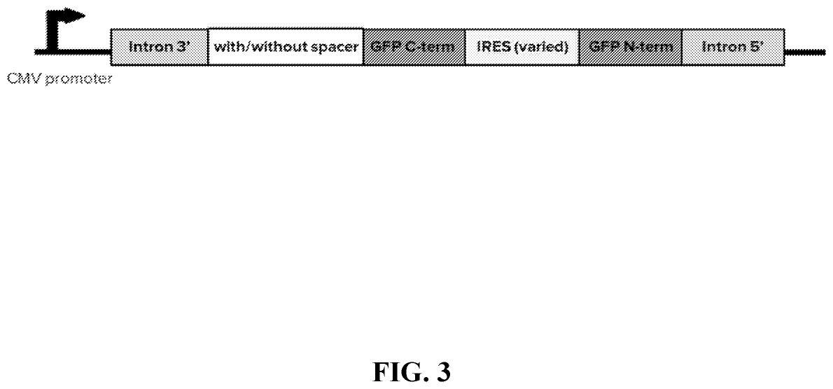

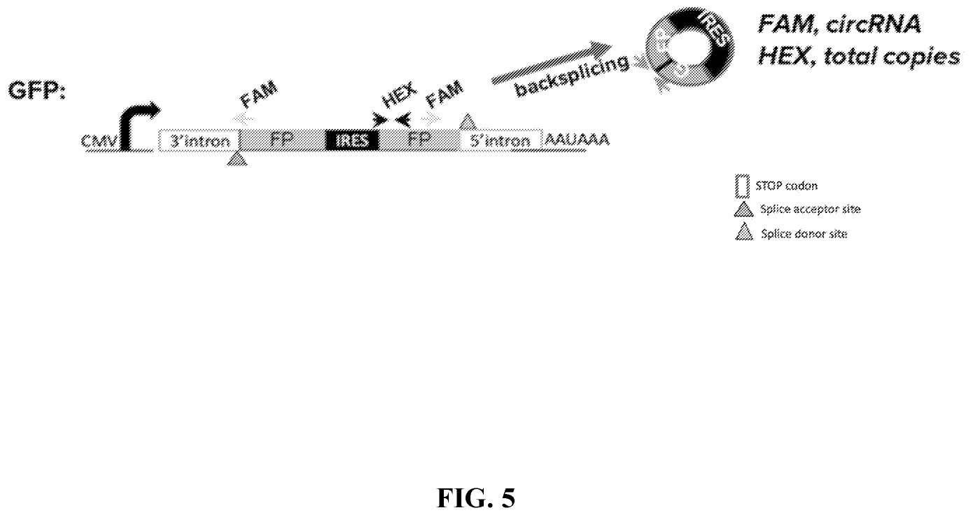

For in-cell circularization, a mammalian expression vector, pTwist_CMV (see FIG. 3), was modified to include a payload consisting of an mRuby fluorescent reporter (to be used as a control for transfection), with UTR sequences enabling standard cap-dependent translation. Downstream of the mRuby stop codon, a split reporter payload consisting of an IRES, a split GFP ORF, and a permuted HIPK3 intron was inserted, with GFP's c terminus first, so that only backspliced circRNA would contain a viable GFP ORF (FIG. 5). Constructs were constructed as a pool varying only by their IRES. Sequences were inserted into the introns or exons using PCR and restriction cloning.

After transcription, the pre-mRNA of the construct will undergo spliceosome mediated back-splicing and reconstitute full-length eGFP on the circRNA. (See FIG. 5) Because full-length eGFP is only reconstituted upon back-splicing, the eGFP fluorescence signal can only come from the circRNA through cap-independent translation. eGFP fluorescence thus serves as a measure of circularization of the construct.

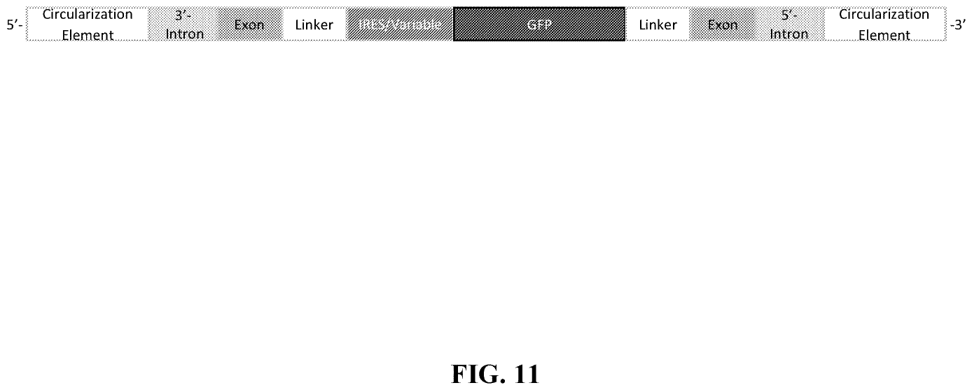

For in vitro RNA generation, constructs were designed with split, permuted type I intron halves (T4 or Anabaena) on either side of an IRES and a GFP reporter (FIG. 11), embedded in a cloning vector, downstream of a T7 promoter element, and upstream of an EcoRV site, to enable in vitro transcription with T7 polymerase. Sequences were cloned into a multiple cloning site of a high copy number plasmid. An EcoRV restriction site was included at the end of the transcript. DNA template for transcription was either PCR-amplified using Q5 DNA polymerase (New England Biolabs) or generated from plasmids digested with EcoRV (New England Biolabs) following manufacturer's instructions.

### Example 2—In Vitro Transcription (IVT)

In vitro transcription reactions were performed at 40° C. for 2 hours, with the following standard reaction composition: 1x RNA Polymerase Reaction Buffer, 10 mM ATP, 10 mM CTP, 10 mM UTP, 10 mM GTP, 5 mM DTT, 5 U/μL T7 RNA Polymerase (NEB, Ipswich, MA), 1 U/μL RNasin® Plus (Promega) and 250 ng double strand DNA template containing the T7 promoter. Transcribed RNA was DNase treated with 1U of DNase I at 37° C. for 15 minutes. RNA transcripts were purified using RNAClean XP (Beckman Coulter). Except where otherwise stated, RNA samples were visualized by size separation on a TapeStation system (Agilent) following manufacturer's instructions. RNA concentration was determined by quantification on a NanoDrop Spectrophotometer (ThermoFisher Scientific, Carlsbad CA).

### Example 3—Circular RNA Generation for Screening in Cells

Unmodified circular RNA was generated in vitro. Unmodified linear RNA was in vitro transcribed. In vitro transcription reactions contained 1 μg of template DNA T7 RNA polymerase promoter, 10× AmpliScribe T7 Flash Reaction Buffer, 7.5 mM ATP, 7.5 mM CTP, 7.5 mM GTP, 7.5 mM UTP, 10 mM DTT, 40U RiboGuard RNase Inhibitor, 2 μl AmpliScribe T7 Enzyme Solution in a 20 μl Total reaction volume. Transcription was carried out at 37° C. for 2 hours. Transcribed RNA was DNase treated with 1U of DNase I at 37° C. for 15 minutes. Re-folding of the RNA after DNase treatment consisted of a 15 minute incubation at 55° C. To favor circularization by self-splicing, additional GTP was added to a final concentration of 2 mM, incubated at 55° C. for 15 minutes. RNA was then column purified and visualized in a 2% E-gel.

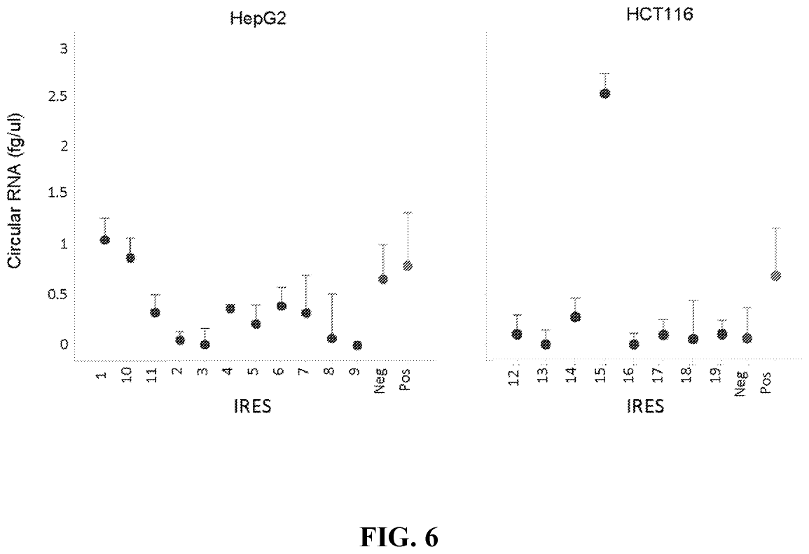

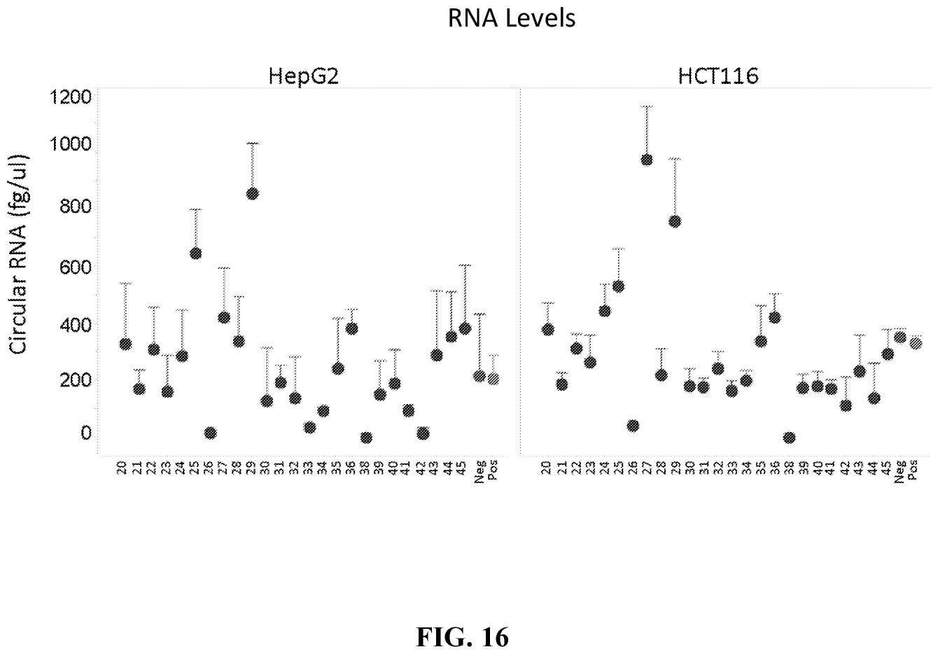

RT-qPCR using Taqman probes labeled with FAM, HEX or Cy5 was employed to measure RNA generated in a cell (FIG. 5). The creation of each new copy increased the intensity of the fluorescent signal that was proportional to the amount of the amplicon in the sample. Samples were quantified by determining the number of cycles it took for the fluorescence to reach a particular threshold. The number (Cq) was inversely proportional to the amount of the target. Target sequences corresponding to GFP (total RNA), circRNA as well as housekeeping gene (GAPDH) were measured. circRNA was measured in absolute concentration (fg/ul). The results of qPCR showed the effect of each IRES on total circular RNA generation (FIG. 6 (DNA constructs) and FIG. 16 (RNA constructs)).

A similar level of circularization was observed across the library, despite IRES sequence heterogeneity for both cell lines (FIG. 6). Thus, observed differences in translation correspond to the IRES sequence rather than to varying levels of circularization of the constructs.

### Example 4—Cell Expression of Circular RNA Encoded Protein

A circular RNA was engineered to express a biologically active protein in cells. To monitor expression of protein from RNA in cells, 5×10³ cells were successfully reverse transfected with a lipid-based transfection reagent (Invitrogen) and 2 nM of linear or circular RNA. GFP was used as the reporter protein to measure translation efficiency of each IRES tested. A CVB3 IRES was uses to drive translation of GFP (positive control, or “pos”) and Gaussia Luciferase (negative control or “neg”).

HepG2 (HB-8065) and HCT 116 (CCL-247) cells from the American Type Culture Collection were maintained with Eagle's Minimum Essential Media (ATCC) and McCoy′s5A (Gibco) and supplemented with 10% FBS (Gibco). For routine subculture, 0.25% TrypLE (Thermo Fisher Scientific) was used for cell dissociation.

HCT116 cells were seeded at 25k/well and HepG2 cells at 32.5k/well.

For in-cell circularization of DNA construct, 200 ng/well (DNA-3:1) DNA plasmid was delivered with TransIT®-2020 Transfection Reagent (Mirus Bio).

For in vitro generated circular RNA using RNA constructs, RNA was delivered with Lipofectamine™ MessengerMax (Invitrogen). The molar amount of mRNA or circRNA delivered was the same for all samples. 0.3 μl of MessengerMax reagent (Invitrogen) was used per 250 ng of RNA and delivered following manufacturer's instructions.

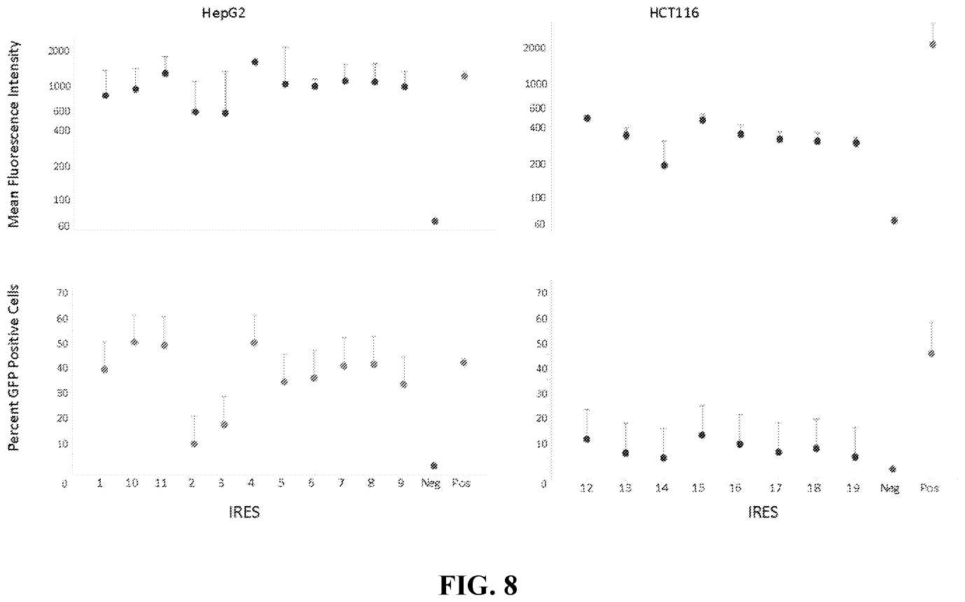

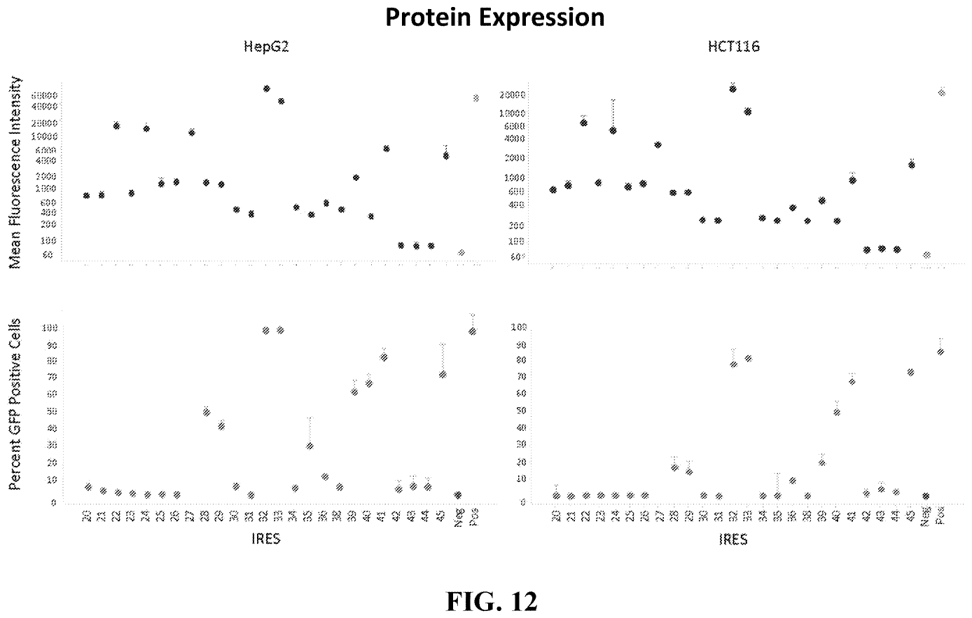

Protein expression was assessed at 48 hours post transfection by flow cytometry (FIG. 8 (DNA constructs) and FIG. 11 (RNA constructs), also showing positive and negative controls) and Circular RNA was quantified by qPCR. RNA molecules were transcribed from DNA templates using New England Biolabs' Hiscribe T7 Quick High Yield RNA synthesis kit. Briefly, 200 ng of DNA template was mixed with nucleotides and T7 polymerase in a 20 μl reaction. The reaction was incubated at 40° C. for 2-3 hours. DNase I (2 μl) was added to quench the remaining DNA template in the reaction by incubation at 37° C. for 20 min. Prior to addition of GTP for facilitating self-splicing of Group I introns in the mRNA synthesized, the mixture is incubated at 50° C. for 15 minutes. Immediately, temperature was increased to 55° C. and GTP was added to the RNA mixture (final concentration ≥2 mM) for 15 minutes. This final reaction was purified by RNA Clean XP magnetic beads using manufacturer's protocol. Transcription products were analyzed by Agilent Tapestation to determine RNA quality and electropherogram profile. A GFP reporter was used to assess translation and additional motifs that can potentially influence translation were also included. The constructs (5 nM/well) were transfected using MessengerMax in an arrayed format into HepG2 (liver, 32k/well) and HCT116 (colon, 25k/well). GFP fluorescence (translational output, FIG. 12) was measured 48 hours later by flow cytometry while RNA levels were assessed by qPCR (FIG. 16).

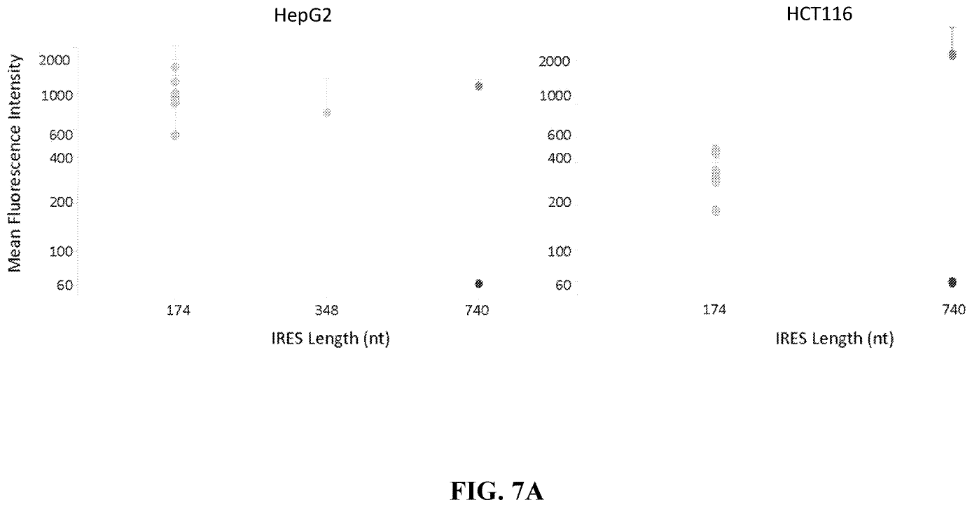

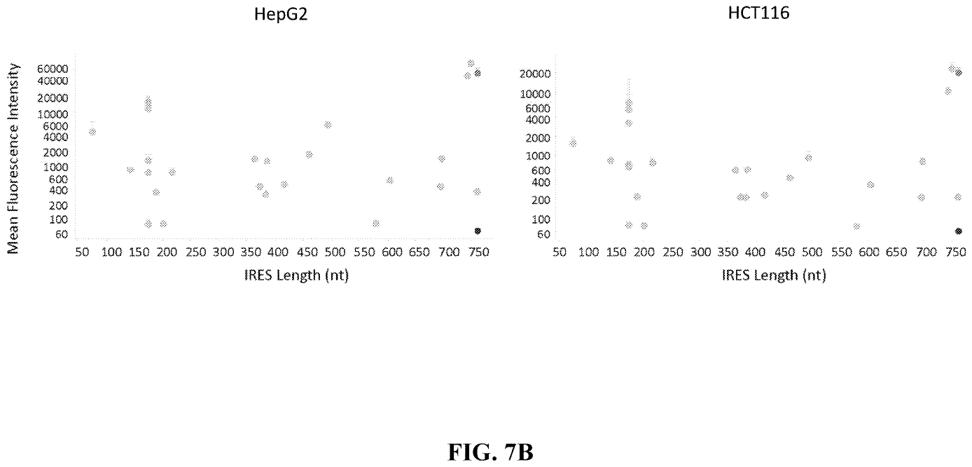

FIG. 7A (DNA constructs) and FIG. 7B (RNA constructs) show the distribution of GFP expression vs IRES length of IRES's in Table 1. Similar expression level is observed from shorter IRESs compared to a longer ~740 nt IRES (CVB3, the positive control) in both cell lines.

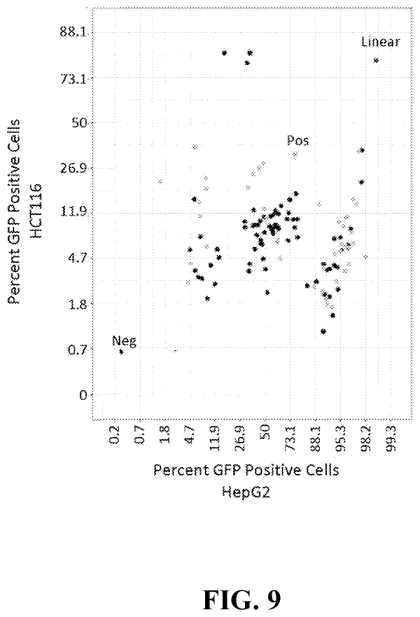

In FIG. 8, the Y-axis represents either percent of cells expressing GFP or Mean Fluorescence Intensity and the x-axis identifies the IRES construct tested. The IRES number in the x-axis corresponds to the matching SEQ. ID in Table 1. Furthermore, in RNA constructs some of the IRESs exhibited transcription activity in the two cell lines tested (FIG. 12), indicating transcription activity across different cell types. However, some IRESs drove translation and protein expression better in one or the other cell line, showing tissue-specificity for those sequences, given the different anatomical origin of the two human cell lines (FIG. 8 and FIG. 9). Thus, sequences that modulate translation can afford additional levels of control over the transcription and expression level of the payload. Sequence-level control, combined with differential expression in tissues, can be leveraged to generate circRNAs tailored to specific therapeutic needs.

### Example 5—Self-Splicing for Intron Screening

Unmodified circular RNA was generated in vitro. Unmodified linear RNA was in vitro transcribed. In vitro transcription reactions contained 1 μg of template DNA T7 RNA polymerase promoter, 10X AmpliScribe T7 Flash Reaction Buffer, 7.5 mM ATP, 7.5 mM CTP, 7.5 mM GTP, 7.5 mM UTP, 10 mM DTT, 40U RiboGuard RNase Inhibitor, 2 μl AmpliScribe T7 Enzyme Solution in a 20 μl Total reaction volume. Transcription was carried out at 37° C. for 4 hours. Transcribed RNA was DNase treated with 1U of DNase I at 37° C. for 15 minutes. To favor circularization by self-splicing, RNA was pre-incubated for 15 minutes at 50° C. Then, additional GTP was added to a final concentration of 2 mM, incubated at 55° C. for 15 minutes. RNA was then column purified and visualized by UREA-PAGE.

### Example 6—Intron Screen with Circular RNA Generated In Vitro

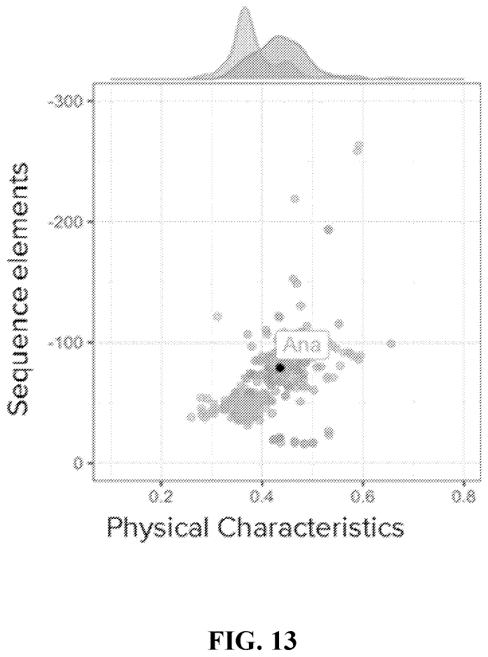

To generate a library of type I introns, several sources and methods were used including databases of functional RNAs, Rfam, the RNA Families Database and mined from different sources across the tree of life. More than 20 thousand potential intron sequences were obtained from the in silico platform. These were filtered using sequence composition and biophysical properties (FIG. 13). These sequences were clustered by sequence similarity, prioritized based on a custom scoring function, and then chimeric sequences were generated in silico (FIG. 13). The introns for testing were also split into two parts, designated A (5′ half) and B (3′ half). These sequences were included in an arrayed screen for type I introns.

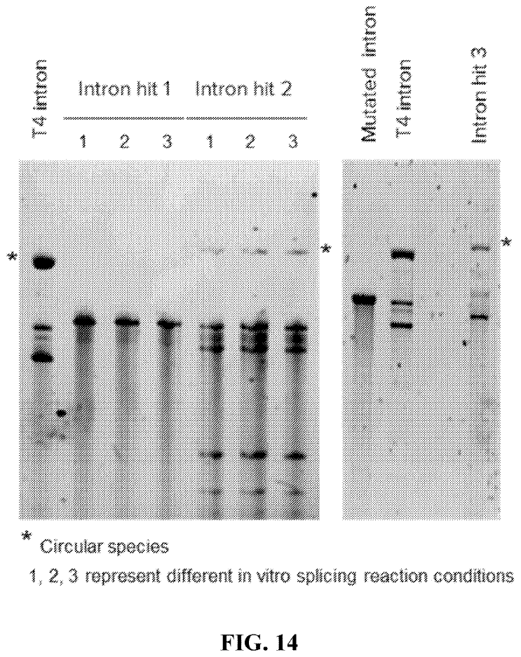

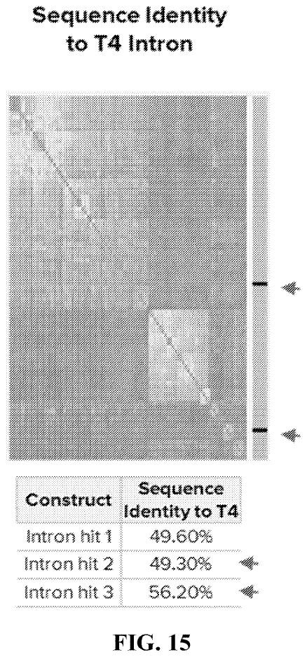

Selected intron sequences were tested for in vitro splicing (FIG. 14). Polyacrylamide gel electrophoresis was used to detect circular forms (marked with an asterisk) (FIG. 14). T4 and a T4 intron mutated to remove its function were used as positive and negative controls. Intron 1 was a negative hit, Intron 2 and 3 resulted in circularization in vitro as indicated by the slower running bands in the gel marked by the asterisk. These introns had low sequence similarity to a previously reported T4 intron (FIG. 15), including both positive and negative hits. The sequence identity of these introns to the T4 intron is less than 60%.

### Example 7—Cell Expression of a Protein Encoded in a Partially Modified Circular RNA

Non-naturally occurring circular RNAs were engineered to express a biologically active intracellular protein in cells.

The circular RNA used for these experiments was generated as previously described in Examples 2 and 4 except that a 0.25:1 ratio for N1-methyl PseudoUTP to UTP was used. This amount of N1-methyl PseudoUTP results in 25% incorporation of the modified UTP per RNA. Briefly, RNA molecules were transcribed from DNA templates using Hiscribe T7 Quick High Yield RNA synthesis kit (New England Biolabs). 400 ng of DNA template was mixed with 10 mM ATP, 10 mM CTP, 10 mM GTP, 7.5 mM UTP, 2.5 mM N1-methyl PseudoUTP (Apexbio Technology, cat. #NC1948878), and 5 U/μL T7 RNA Polymerase in a 20 μl reaction. The reaction was incubated at 40° C. for 2 hours. Transcribed RNA was DNase treated with 1U of DNase I at 37° C. for 15 minutes to remove the double stranded DNA template. Prior to addition of GTP for facilitating self-splicing of Group I introns in the synthesized RNA, the mixture was incubated at 50° C. for 15 minutes. Immediately, temperature was increased to 55° C. and GTP was added to the RNA mixture (final concentration ≥2 mM) for 15 minutes. This final reaction was purified by RNA Clean XP magnetic beads using manufacturer's protocol. RNA concentration was determined by quantification on a NanoDrop Spectrophotometer (ThermoFisher Scientific, Carlsbad CA) and it is normalized to the desired transfection concentration.

Flow cytometry was performed to assess protein expression in HepG2 and HEK293T cells 48 hours post reverse transfection of 3×10⁴ cells with 0.3 μl of Lipofectamine™ MessengerMAX™ (Invitrogen LMRNA001), a lipid-based transfection reagent and 6 nM of linear or circular RNA following manufacturer's instructions.

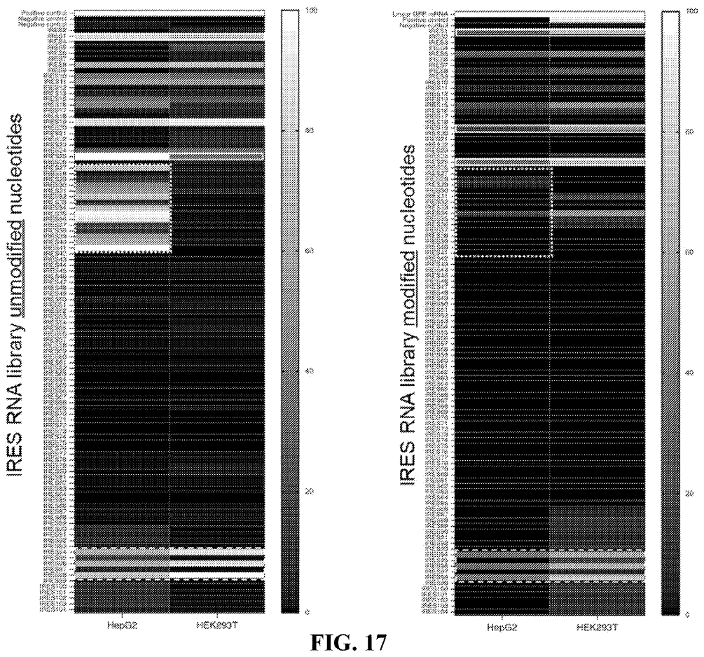

FIG. 17 shows the distribution of GFP expression in Hek293T and HepG2 cells transfected with unmodified and modified IRES RNA libraries. It is expected that RNA modifications interfere with RNA structure and hence with IRES function. Here, RNA modification resulted in significantly decreased GFP expression driven by several IRESs in HepG2 cells (white square-short dashed line). And a slight decrease in expression was observed for a subset of IRES in both Hek293T and HepG2 cells (white square-continuous line). Notably, several IRES elements kept their translation driver function when 25% of the uracils in the circular RNA were modified with N1-methyl PseudoUTP (white square-long dashed line) (FIG. 17 and Table 5).

TABLE 5
Translation functional active IRES in the context of
modified circular RNAs transfected in HepG2 cells

| IRES # | Name | Size |
|---|---|---|
| IRES1 | CVB | 741 |
| IRES5 | RF00210_35 | 462 |
| IRES19 | AEV | 494 |
| IRES25 | PV_type3_Leon | 742 |
| IRES94 | CVB3 | 750 |
| IRES96 | EMCV | 696 |
| IRES98 | EV71_1-748 | 748 |

The ability of IRESs to drive translation in the context of a modified RNA, like nucleoside methylation, open the doors to the next generation of more stable, immune-silent and long-lived protein encoding RNAS for therapeutic use.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

What is claimed is:

1. A nucleic acid molecule comprising an internal ribosomal entry site (IRES) sequence comprising any one of the nucleic acid sequences listed in Table 1.

2. The nucleic acid molecule of claim 1, wherein the IRES sequence is operably linked to a protein coding sequence.

3. The nucleic acid molecule of claim 2, wherein the protein coding sequence encodes a therapeutic protein or polypeptide.

4. The nucleic acid molecule of any one of claims 1-3, further comprising an optional spacer sequence situated 3′ to the IRES sequence or between the IRES sequence and the protein coding sequence.

5. The nucleic acid molecule of any one of claims 1-4, further comprising a translation regulation motif.

6. The nucleic acid molecule of any one of claims 1-5, further comprising a reporter sequence.

7. The nucleic acid molecule of any one of claims 1-6, wherein the nucleic acid molecule is an RNA molecule.

8. The nucleic acid molecule of any one of claims 1-6, wherein the nucleic acid molecule is a DNA molecule.

9. The nucleic acid molecule of any one of claims 1-8, further comprising a 5′ intron sequence and a 3′ intron sequence.

10. The nucleic acid molecule of claim 9, wherein the 5′ intron sequence is an initial intron sequence or a listed in Table 3.

11. The nucleic acid molecule of claim 10, wherein the 3′ intron sequence is a corresponding intron sequence listed in Table 3.

12. The nucleic acid molecule of claim 9, wherein the 5′ intron sequence is a corresponding intron sequence or a listed in Table 3.

13. The nucleic acid molecule of claim 10, wherein the 3′ intron sequence is an initial intron sequence listed in Table 3.

14. The nucleic acid molecule of any one of claims 9-13, wherein the 5′ intron and the 3′ intron are self-splicing elements that together are capable of mediating splicing that produces a circular nucleic acid molecule from a linear nucleic acid.

15. The nucleic acid molecule of any one of claims 1-12, wherein the nucleic acid molecule is a circular nucleic acid molecule.

16. The nucleic acid molecule of any one of claims 1-15, wherein the nucleic acid molecule is a linear nucleic acid molecule.

17. A nucleic acid molecule comprising an initial intron sequence listed in Table 3 and a corresponding intron sequence listed in Table 3.

18. The nucleic acid molecule of claim 17, further comprising an IRES sequence.

19. The nucleic acid molecule of claim 18, wherein the IRES sequence comprises any one of the nucleic acid sequences listed in Table 1.

20. The nucleic acid molecule of claim 18 or 19, wherein the IRES sequence is operably linked to a protein coding sequence.

21. The nucleic acid molecule of claim 20, wherein the protein coding sequence encodes a therapeutic protein or polypeptide.

22. The nucleic acid molecule of any one of claims 17-21, further comprising a spacer sequence.

23. The nucleic acid molecule of any one of claims 17-22, further comprising a translation regulation motif.

24. The nucleic acid molecule of any one of claims 17-23, further comprising a reporter sequence.

25. The nucleic acid molecule of any one of claims 17-24, wherein the nucleic acid molecule is an RNA molecule.

26. The nucleic acid molecule of any one of claims 17-24, wherein the nucleic acid molecule is a DNA molecule.

27. The nucleic acid molecule of any one of claims 17-26, wherein the initial intron and the corresponding intron are self-splicing elements that together are capable of mediating splicing that produces a circular nucleic acid molecule from a linear nucleic acid.

28. The nucleic acid molecule of any one of claims 17-27, wherein the nucleic acid molecule is a circular nucleic acid molecule.

29. A nucleic acid molecule comprising, in 5′ to 3′ order:
(i) a 5′ intron sequence,
(ii) an IRES sequence comprising any one of the nucleic acid sequences listed in Table 1, and
(iii) a 3′ intron sequence.

30. The nucleic acid molecule of claim 29, wherein:
(a) the 5′ intron sequence comprises any one of the initial intron sequences listed in Table 3 and the 3′ intron sequence comprises any one of the corresponding intron sequences listed in Table 3; or
(b) the 5′ intron sequence comprises any one of the corresponding intron sequences listed in Table 3 and the 3′ intron sequence comprises one of the initial intron sequences listed in Table 3.

31. A nucleic acid molecule comprising, in 5′ to 3′ order:
(i) a 5′ intron sequence comprising any one of the initial intron sequences listed in Table 3,
(ii) an IRES sequence, and
(iii) a 3′ intron sequence comprising any corresponding intron sequence listed in Table 3.

32. A nucleic acid molecule comprising, in 5′ to 3′ order:
(i) a 5′ intron sequence comprising any one of the corresponding intron sequences listed in Table 3,
(ii) an IRES sequence, and
(iii) a 3′ intron sequence comprising any one of the initial intron sequence listed in Table 3.

33. The nucleic acid molecule of claim 31 or claim 32, further comprising a protein coding sequence between (ii) and (iii).

34. The nucleic acid molecule of claim 331, wherein the IRES sequence is operably linked to the protein coding sequence.

35. The nucleic acid molecule of claim 33 or 34, wherein the protein coding sequence encodes a therapeutic protein or polypeptide.

36. The nucleic acid molecule of any one of claims 33-35, further comprising one or more spacer sequences between (i) and (iii); between (i) and (ii), between (ii) and (iii); between (ii) and the protein coding sequence; and/or between the protein coding sequence and (iii).

37. The nucleic acid molecule of any one of claims 31-36, further comprising a reporter sequence between (i) and (iii).

38. The nucleic acid molecule of any one of claims 31-37, wherein the nucleic acid molecule is an RNA molecule.

39. The nucleic acid molecule of any one of claims 31-37, wherein the nucleic acid molecule is a DNA molecule.

40. The nucleic acid molecule of any one of claims 31-39, wherein the 5′ intron and the 3′ intron are self-splicing elements that together are capable of mediating splicing that produces a circular nucleic acid molecule from a linear nucleic acid.

41. The nucleic acid molecule of any one of claims 31-40, wherein the nucleic acid molecule is a circular nucleic acid molecule.

42. The nucleic acid molecule of any one of claims 31-39, wherein the nucleic acid molecule is a linear nucleic acid molecule.

43. A construct comprising the nucleic acid molecule of any one of claims 1-42.

44. A circular nucleic acid molecule produced by any one of the nucleic acid molecules according to any one of claims 1-41.

45. A cell comprising the nucleic acid molecule of any one of claims 1-42, the construct of claim 43, or the circular nucleic acid molecule of claim 44.

46. A method of generating circular nucleic acid molecules, the method comprising expressing the nucleic acid molecule of any one of claims 1-42, the construct of claim 43, or the circular nucleic acid molecule of claim 44 in a cell.

47. A method of expressing a protein in a cell comprising contacting the cell with the nucleic acid molecule of any one of claims 1-42, the construct of claim 43, or the circular nucleic acid molecule of claim 44.


SEQUENCE LISTING (54 sequences)

SEQ ID NO: 1 - DNA, 348 bp, synthetic construct
  misc_feature 1..348: note = Synthetic
  source 1..348: mol_type = other DNA
taacacccgtagctttgaaatacggctcgctctttgaatatcctagaaacctttgaagctctatatcacctttgaacgaaactctcctctttgaatccccgccagctctttgaataatcatgactgctttgaaagtctgcttcctctttgaattaaatatttttctttgaattacctattcggctttgaatacttctttctactttgaacgttattttgctctttgaatcggcctcttttctttgaatgaaaccatactctttgaatgactttttaatctttgaaatcaaacctttactttgaattgaatatatctctttgaaatttccgactcggaccgatggtgag

SEQ ID NO: 2 - DNA, 174 bp, synthetic construct
  misc_feature 1..174: note = Synthetic
  source 1..174: mol_type = other DNA
cctttgaaacatgtcctttaacacgttgaactcgtatataattgttttctttgaagtcaaagcatagtatgccttctttttatatctttcaagcgctttgaactcagtgcagactcgtttcttatcatatgccgtctattaactttgaaatttccgactcggaccgatggtgag

SEQ ID NO: 3 - DNA, 174 bp, synthetic construct
  misc_feature 1..174: note = Synthetic
  source 1..174: mol_type = other DNA
agagtctcgcatcgttagctagagtctcgttttatacctaagagtctcgttacctgacttagagtctcgttctattttctagagtctcgccacttattttagagtctcgaagcttatgttagagtctcgattttgtggtaagagtctcgatttccgactcggaccgatggtgag

SEQ ID NO: 4 - DNA, 174 bp, synthetic construct
  misc_feature 1..174: note = Synthetic
  source 1..174: mol_type = other DNA
gacaaggtgttacaaacatacgcggattccatagcagaatttatgacacccagtgcccagaagaaggagctgtacctccagaaccactatgatgctgttgaggctggacttaattcaatagctgcagcatggaaagacttggccaacaaatgctatgcttactcccgacccgaa

SEQ ID NO: 5 - DNA, 174 bp, synthetic construct
  misc_feature 1..174: note = Synthetic
  source 1..174: mol_type = other DNA
tgctttcgttcgttttttgttatttaatgattcttcgtcttgtaaaacctttcgttctgtatatagacttcccccctttctctgcttattgttcctttcgttccacgttatttttcttcctgtgtactgcagtttacttcctttcgttcatttccgactcggaccgatggtgag

SEQ ID NO: 6 - DNA, 174 bp, synthetic construct
  misc_feature 1..174: note = Synthetic
  source 1..174: mol_type = other DNA
tttatgatttcaacatggatacatatttcagttctttctttttctcactatctgaaaatattagagtctcgactaaatcttgtgatttagagtctcgtgtctgtgggatgcgatagagtctcggagcccttaatgtgcttagagtctcgatttccgactcggaccgatggtgag

SEQ ID NO: 7 - DNA, 174 bp, synthetic construct
  misc_feature 1..174: note = Synthetic
  source 1..174: mol_type = other DNA
tttatgatttcaacatggatacatatttcagttctttctttttctcactatctgaaaatattagagtctcggtctctcgccttatataagagtctcgacaagccccttactttaagagtctcgtttcgttgtaggattttagagtctcgatttccgactcggaccgatggtgag

SEQ ID NO: 8 - DNA, 174 bp, synthetic construct
  misc_feature 1..174: note = Synthetic
  source 1..174: mol_type = other DNA
agagtctcgaaataaaagtgagagtctcgatattatctttagagtctcgcaattttttcaagagtctcgttgttttttcaagagtctcgctctttactttagagtctcgaaactatgctaagagtctcgatcacatgcatagagtctcgatttccgactcggaccgatggtgag

SEQ ID NO: 9 - DNA, 174 bp, synthetic construct
  misc_feature 1..174: note = Synthetic
  source 1..174: mol_type = other DNA
tttatgatttcaacatggatacatatttcagttctttctttttctcactatctgaaaataccggccgggtacaccattataattacgcctatactcgagaccggccgggtttgttcttcagacttttttatctaccctcaccggccgggatttccgactcggaccgatggtgag

SEQ ID NO: 10 - DNA, 174 bp, synthetic construct
  misc_feature 1..174: note = Synthetic
  source 1..174: mol_type = other DNA
ctctttcgttcttcgtatagatgggggttttatctctcttatgttcccctttcgttccatttcttaatcgctagtccactaaattattgctctactttcgttctatttatgtaaatgattcatttcctagactgattacactttcgttcatttccgactcggaccgatggtgag

SEQ ID NO: 11 - DNA, 174 bp, synthetic construct
  misc_feature 1..174: note = Synthetic
  source 1..174: mol_type = other DNA
tgacacaattttgcaatgacatgaagttgttgaatggaattagggctggcaattatacatttattcctactccgaaaattatatgtgcatatggtactcctgaaatatgtttgagagcccctgagatacgatcatttggtagatatttaccaattgacaaggatgaatactctg

SEQ ID NO: 12 - DNA, 174 bp, synthetic construct; identical to SEQ ID NO: 3
  misc_feature 1..174: note = Synthetic
  source 1..174: mol_type = other DNA

SEQ ID NO: 13 - DNA, 174 bp, synthetic construct
  misc_feature 1..174: note = Synthetic
  source 1..174: mol_type = other DNA
atgctgctactatctagttctataagcttatataaagacagaaatgaagcaagactgatttcttcacagaataatgccggacttgaagattttgaaaggaaaaaaacccttggaacaggttcatttggaagagtcatgttggtaaaacacaaagccactgaacagtattatgcc

SEQ ID NO: 14 - DNA, 174 bp, synthetic construct
  misc_feature 1..174: note = Synthetic
  source 1..174: mol_type = other DNA
gcggcgcaatacgaatgccccatacatctgatgcggcgcaatacgaatgcccctcttacaaaacgcggcgcaatacgaatgcccctgttccttcaagcggcgcaatacgaatgcccccttgcctaggtgcggcgcaatacgaatgccccatttccgactcggaccgatggtgag

SEQ ID NO: 15 - DNA, 174 bp, synthetic construct
  misc_feature 1..174: note = Synthetic
  source 1..174: mol_type = other DNA
tttatgatttcaacatggatacatatttcagttctttctttttctcactatctgaaaataagagtctcggggtatcaattatcactgttcacgatttataagagtctcggttttttttatgatttccacctgcatgcttaagagtctcgatttccgactcggaccgatggtgag

SEQ ID NO: 16 - DNA, 174 bp, synthetic construct
  misc_feature 1..174: note = Synthetic
  source 1..174: mol_type = other DNA
caataaagtatttaaagctgggtataaaggagggatttgggcttttgttgtgagcatcaccaacacgtacatctacgcgtacctgacatggccgcgcccaggggcagacccaaaaaggaccttacgatggaagacctaaccgcaaagataagccaattgactgtggagaatcgg

SEQ ID NO: 17 - DNA, 174 bp, synthetic construct; identical to SEQ ID NO: 5
  misc_feature 1..174: note = Synthetic
  source 1..174: mol_type = other DNA

SEQ ID NO: 18 - DNA, 174 bp, synthetic construct; identical to SEQ ID NO: 8
  misc_feature 1..174: note = Synthetic
  source 1..174: mol_type = other DNA

SEQ ID NO: 19 - DNA, 174 bp, synthetic construct
  misc_feature 1..174: note = Synthetic
  source 1..174: mol_type = other DNA
gcggcgcaatacgaatgccccagtccttctctatctcgcctactaaatgttttatttatttttttaaattccacctgtcctatacgattcttgcatcctagtttacctccgtaattgttgaaatgtacgcggcgcaatacgaatgccccatttccgactcggaccgatggtgag

SEQ ID NO: 20 - DNA, 174 bp, synthetic construct
  misc_feature 1..174: note = Synthetic
  source 1..174: mol_type = other DNA
gtactccctttctgattcttttagctaactatgcgtaagctattcgattactcccatctttacaattactagtttcatctggcccctgcataatatactcccagtaaccaatctgcaaacacaccaaataagttaaaatccctactcccatttccgactcggaccgatggtgag

SEQ ID NO: 21 - DNA, 217 bp, synthetic construct
  misc_feature 1..217: note = Synthetic
  source 1..217: mol_type = other DNA
tgagctgatcgttgttgttattcttcaatttctgcaggtaacttaggaagcgtttcttgagcttccctgggatgagcgtttgggagagctgattctgcagcccagagagtagtctcagggcatcctctggagcctgacctgtgaccgtcgcatcatagaccgccagtagacctgggagcagattcaccgccgcggccgtctcctttaaggtgctgtg

SEQ ID NO: 22 - DNA, 174 bp, synthetic construct
  misc_feature 1..174: note = Synthetic
  source 1..174: mol_type = other DNA
tttatgatttcaacatggatacatatttcagttctttctttttctcactatctgaaaatagtccggccgggttctctcaaactcgttaccggccggggagttttctacctatatccggccgggatattcttggaagctttccggccgggatttccgactcggaccgatggtgag

SEQ ID NO: 23 - DNA, 142 bp, synthetic construct
  misc_feature 1..142: note = Synthetic
  source 1..142: mol_type = other DNA
gtttccactgctgctctgagggcacaagaggcaggatttggaattcctggagaactgtcttcgtgagaagctggaaatatttctttcaattccatctcttagttttccattttgttgtgtttcagaggaacatcaagaaacc

SEQ ID NO: 24 - DNA, 174 bp, synthetic construct
  misc_feature 1..174: note = Synthetic
  source 1..174: mol_type = other DNA
ccggcgggtattcttatattccggcgggtttatacatcgcccggcgggtaagtgtttcttccggcgggtttgcttatgttccggcgggtcatgactttccccggcgggtgacatcctgttccggcgggtttttcatatatccggcgggtatttccgactcggaccgatggtgag

SEQ ID NO: 25 - DNA, 174 bp, synthetic construct
  misc_feature 1..174: note = Synthetic
  source 1..174: mol_type = other DNA
tttatgatttcaacatggatacatatttcagttctttctttttctcactatctgaaaatagaagctgcccggcgggtcatggcgccattcagttaattccctcctgttctcgcgttttattactctttgaagctgcccggcgggtcatgatttccgactcggaccgatggtgag

SEQ ID NO: 26 - DNA, 696 bp, synthetic construct
  misc_feature 1..696: note = Synthetic
  source 1..696: mol_type = other DNA
ttttctttgaatcttttggtttctctttgaaccctcttgtctttctttgaattctttataatgtctttgaaaaagcttctttgtctttgaattcatttcgctgtctttgaagtcattaaagccgctttgaattttaatccgaacctttgaatttctaccaatcgctttgaaatcttatttactgctttgaattttctttttcctctttgaattctgtgttttatctttgaagtgggcattatgcctttgaacagactctagcaactttgaattctgccaatggtctttgaatgcgcatattttcctttgaattctatttatttactttgaatcccttaacagagctttgaaagcataccccaatctttgaatttatatttttttctttgaacacagtatcttttctttgaaatatcaaaggcccctttgaattccccctttatactttgaacgtcttttacaacctttgaaactcacatcttatctttgaaccaacgctcttaactttgaatcttctatcagatctttgaatttgtccttacccctttgaaacgatccaccaatctttgaatttgtctgccgatctttgaatttcacgatcggtctttgaagagtagtcacttgctttgaatatatgagctcctctttgaacatagatcattttctttgaaatttccgactcggaccgatggtgag

SEQ ID NO: 27 - DNA, 174 bp, synthetic construct
  misc_feature 1..174: note = Synthetic
  source 1..174: mol_type = other DNA
gggactatgttttgaaactgacttccagagctccactgcctccctgcaccatgccctacaacttctgcctgcccagtaatgtgcctgcacccagctgctcctcccggccctgtgtgccccccagctgccacggctacaccctgcccggggcctgcaacatccctgccaatgtga

SEQ ID NO: 28 - DNA, 363 bp, synthetic construct
  misc_feature 1..363: note = Synthetic
  source 1..363: mol_type = other DNA
atacgtattgggcaattaaaaataataattaggcctagggaacaaatccctctcagcgaaggccgaaaagaggctagccatgcccttagtaggactagcataatgaggggggtagcaacagtggtgagttcgttggatggcttaagccctgagtacagggtagtcgtcagtggttcgacgccttggaataaaggtctcgagatgccacgtggacgagggcatgcccaaagcacatcttaacctgagcgggggtcgcccaggtaaaagcagttttaaccgactgttacgaatacagcctgatagggtgctgcagaggcccactgtattgctactaaaaatctctgctgtacatggcacatggag

SEQ ID NO: 29 - DNA, 385 bp, synthetic construct
  misc_feature 1..385: note = Synthetic
  source 1..385: mol_type = other DNA
gtatacgagaattagaaaaggcactcgtatacgtattgggcaattaaaaataataattaggcctagggaacaaatccctctcagcgaaggccgaaaagaggctagccatgcccttagtaggactagcataatgaggggggtagcaacagtggtgagttcgttggatggcttaagccctgagtacagggtagtcgtcagtggttcgacgccttggaataaaggtctcgagatgccacgtggacgagggcatgcccaaagcacatcttaacctgagcgggggtcgcccaggtaaaagcagttttaaccgactgttacgaatacagcctgatagggtgctgcagaggcccactgtattgctactaaaaatctctgctgtacatggcac

SEQ ID NO: 30 - DNA, 373 bp, synthetic construct
  misc_feature 1..373: note = Synthetic
  source 1..373: mol_type = other DNA
gtatacgaggttagttcattctcgtatacacgattggacaaatcaaaattataatttggttcagggcctccctccagcgacggccgaactgggctagccatgcccatagtaggactagcaaaacggagggactagccatagtggcgagctccctgggtggtctaagtcctgagtacaggacagtcgtcagtagttcgacgtgagcagaagcccacctcgagatgctacgtggacgagggcatgccaagacacaccttaaccctagcgggggtcgctagggtgaaatcacaccacgtgatgggagtacgacctgatagggcgctgcagaggcccactattaggctagtataaaaatctctgctgtacatggcac

SEQ ID NO: 31 - DNA, 759 bp, synthetic construct
  misc_feature 1..759: note = Synthetic
  source 1..759: mol_type = other DNA
attgcttttcttccaactaaacccctattgatgtgttggtcgtttgccaatcggagggcgacaggcacaggtcgctccgagttctagtagtgtgggaacttgttactactgatgaaacgaggtagtgacactcagtacctgcgaacgaggtcggggccctcccttcttccttcacccaactttcacttttcgttccactttagcaggggtcttctttctatccccctggcggcattggaactagccgtcgcgtcttaacgcgcagccctgaaggccccacaccttgtggatcttgccgtgggtatgtttctggcatgtgtttctcaagcctgcaaccgaagccgaacagccacatgaacagtttgagcgtggtagcgctgtgtgagttggcggtggatccccctcgtggtaacacgagcccccgtggccaaaagcccagtgtttacagcacctctcacatccaggacgaccccatcctggcgctcactcttagtagtatggcttagtacgcattaggtggtaagccgagctctccctcggccttgttctgaatgcacacatgtctaggggctaaggatgtcctacaggtacccgcacgtaaccttcagagagtgcggatctgagtaggagaccgtggtgcactgctttacagatgcagcccggtttaaaaagcgtctatgcccctacagggtagcggtgggccgcgccctttccttttaaaactacttgttctatggtgacaatggcaggaaacatgat

SEQ ID NO: 32 - DNA, 748 bp, synthetic construct
  misc_feature 1..748: note = Synthetic
  source 1..748: mol_type = other DNA
ttaaaacagcctgtgggttgcacccacccacagggcccactgggcgctagcactctggtactgaggtacctttgtgcgcctgtttttactccccttcccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagagcctattgagctagctggtagtcctccggcccctgaatgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaattgtttacctatttattggttttgtaccattatcactgaagtctgtgatcactctcaaattcattttgaccctcaacacaatcaaacatg

SEQ ID NO: 33 - DNA, 742 bp, synthetic construct
  misc_feature 1..742: note = Synthetic
  source 1..742: mol_type = other DNA
ttaaaacagctctggggttgttcccaccccagaggcccacgtggcggctagtacactggtatcacggtacctttgtacgcctgttttatactccctcccccgcaacttagaagcatacaattcaagctcaataggagggggtgcaagccagcgcctccgtgggcaagcactactgtttccccggtgaggccgcatagactgttcccacggttgaaagtggccgatccgttatccgctcatgtacttcgagaagcctagtatcgctctggaatcttcgacgcgttgcgctcagcactcaaccccggagtgtagcttgggccgatgagtctggacagtccccactggcgacagtggtccaggctgcgctggcggcccacctgtggcccaaagccacgggacgctagttgtgaacagggtgtgaagagcctattgagctacatgagagtcctccggcccctgaatgcggctaatcctaaccatggagcaggcagctgcaacccagcagccagcctgtcgtaacgcgcaagtccgtggcggaaccgactactttgggtgtccgtgtttccttttattcttgaatggctgcttatggtgacaatcatagattgttatcataaagcgagttggattggccatccagtgtgaatcagattaattactcccttgtttgttggatccactcccgaaacgttttactccttaacttattgaaattgtttgaagacaggatttcagtgtcaca

SEQ ID NO: 34 - DNA, 416 bp, synthetic construct
  misc_feature 1..416: note = Synthetic
  source 1..416: mol_type = other DNA
gctaggaatgctcctggagcaccccccctagcagggcgtgggggatttcccctgcccgtctgcagaagggtggagccaaccaccttagtatgtaggcggcgggactcatgacgctcgcgtgatgacaagcgccaagcttgacttggatggccctgatgggcgttcatgggttcggtggtggtggcgctttaggcagcctccacgcccaccacctcccagatagagcggcggcactgtagggaagaccggggaccggtcactaccaaggacgcagacctctttttgagtatcacgcctccggaagtagttgggcaagcccacctatatgtgttgggatggttggggttagccatccataccgtactgcctgatagggtccttgcgaggggatctgggagtctcgtagaccgtagcac

SEQ ID NO: 35 - DNA, 188 bp, synthetic construct
  misc_feature 1..188: note = Synthetic
  source 1..188: mol_type = other DNA
gactaagtgtgaacttgcctctctcaacaaaaagccaccgacattaagagagagagtattgctgcttagttaactgcaggccctatttagggttaccgcccaggatctgcaacagcattcctgtatcatccagggcaccggtggacttggtgaggattgagttgacctcatcattagagaccagactc

SEQ ID NO: 36 - DNA, 604 bp, synthetic construct
  misc_feature 1..604: note = Synthetic
  source 1..604: mol_type = other DNA
atccaggttgttcccacctggatttcccacagggagtggtactctgttattacggtaactttgtacgccagttttatctcccttcccccatgtaacttagaagtttttcacaaagaccaatagccggtaatcagccagattactgaaggtcaagcacttctgtttccccggtcaatgttgatatgctccaacagggcaaaaacaactgcgatcgttaaccgcaaagcgcctacgcaaagcttagtagcatctttgaaatcgtttggctggtcgatccgccatttcccctggtagacctggcagatgaggctagaaataccccactggcgacagtgttctagcctgcgtggctgcctgcacaccctatgggtgtgaagccaaacaatggacaaggtgtgaagagccccgtgtgctcgctttgagtcctccggcccctgaatgtggctaaccttaaccctgcagctagagcacgtaacccaatgtgtatctagtcgtaatgagcaattgcgggatgggaccaactactttgggtgtccgtgtttcactttttcctttatatttgcttatggtgacaatatatacaatatatatattggcaccatgg

SEQ ID NO: 37 - DNA, 694 bp, synthetic construct
  misc_feature 1..694: note = Synthetic
  source 1..694: mol_type = other DNA
caaaattgcgtgcgagaaagcacgcaaatcaaagtctagtgcgtaattcactcactaccggcgtaatttgtggttatgctattgcgttgagagtgttgtgggcgtagtacgggggcttttggtttgtgtgtgattaatatgcattcccagttttgtttagttttagattactgatttatttttcgaactacccgaatttatttaggctcttcgagaaataatgatgaactgtcctcaacaagtatgaaaagcaattatttgtgaagttacttgttgttgtaagattattgacctcttagatttttctaagttgtaatgctttgttttctgattgactagattatgaatccaattaaaaggagtagtggtctaatatagtctgtgtgacctgcaggcattttgtgaaaagggtaaagtatgaaagctactctcagaaaagtacttatgtattgatgagagccttaaaatgactttatattcacaaaactgctggaagacaatgatctggggtatcacattccctcttaggttaagtttcgcactactaggaatttttgcgaaaattcttaattccattcttatggtgtagtgtttgttcttttcaattggggcgtatctctctttcgcagagaccctggcccaaccctgaaatgaataaaattttacaaaaattcatcgaaaatcgaccttctag

SEQ ID NO: 38 - DNA, 461 bp, synthetic construct
  misc_feature 1..461: note = Synthetic
  source 1..461: mol_type = other DNA
agcaggtttccccaactgacacaaaacgtgcaacttgaaactccgcctggtctttccaggtctagaggggtaacactttgtactgtgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaatgaccct

SEQ ID NO: 39 - DNA, 383 bp, synthetic construct
  misc_feature 1..383: note = Synthetic
  source 1..383: mol_type = other DNA
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaac

SEQ ID NO: 40 - DNA, 494 bp, synthetic construct
  misc_feature 1..494: note = Synthetic
  source 1..494: mol_type = other DNA
tttgaaagaggcctccggagtgtccggaggctctctttcgacccaacccatactggggggtgtgtgggaccgtacctggagtgcacggtatatatgcattcccgcatggcaagggcgtgctaccttgccccttgacgcatggtatgcgtcatcatttgccttggttaagccccatagaaacgaggcgtcacgtgccgaaaatccctttgcgtttcacagaaccatcctaaccatgggtgtagtatgggaatcgtgtatggggatgattaggatctctcgtagagggataggtgtgccattcaaatccagggagtactctggctctgacattgggacatttgatgtaaccggacctggttcagtatccgggttgtcctgtattgttacggtgtatccgtcttggcacactgaaagggtatttttgggtaatcctttcctactgcctgatagggtggcgtgcccggccacgagagattaagggtagcaatttaaac

SEQ ID NO: 41 - DNA, 579 bp, synthetic construct
  misc_feature 1..579: note = Synthetic
  source 1..579: mol_type = other DNA
gataaaagaacctataatcccttcgcacaccgcgtcacaccgcgctatatgctgctcattaggaattacggctccttttttgtggatacaatctcttgtatacgatatacttattgttaatttcattgacctttacgcaatcctgcgtaaatgctggtatagggtgtacttcggatttccgagcctatattggttttgaaaggacctttaagtccctactatactacattgtactagcgtaggccacgtaggcccgtaagatattataactattttattatattttattcaccccccacattaatcccagttaaagctttataactataagtaagccgtgccgaaacgttaatcggtcgctagttgcgtaacaactgttagtttaattttccaaaatttatttttcacaatttttagttaagattttagcttgccttaagcagtctttatatcttctgtatattattttaaagtttataggagcaaagttcgctttactcgcaatagctattttatttattttaggaatattatcacctcgtaattatttaattataacattagctttatctatttata

SEQ ID NO: 42 - DNA, 174 bp, synthetic construct
  misc_feature 1..174: note = Synthetic
  source 1..174: mol_type = other DNA
tcgttatatttagaattagaatgtttcttagcggtcgtgtagttatttttatgtcataagtggataatttgttagctcctataacaaaagtctgttgcttgtgtttcacattttggatttcctaatataatgttctctttttagaaaaggtggacaagtcctattttcaagaga

SEQ ID NO: 43 - DNA, 201 bp, synthetic construct
  misc_feature 1..201: note = Synthetic
  source 1..201: mol_type = other DNA
gcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctacatttcaa

SEQ ID NO: 44 - DNA, 75 bp, synthetic construct
  misc_feature 1..75: note = Synthetic
  source 1..75: mol_type = other DNA
ttcgtttgctttttgtagtataattaaatatttgtcagataagagattgtttagagatttgttctttgtttgata

SEQ ID NO: 45 - DNA, 161 bp, synthetic construct
  misc_feature 1..161: note = Synthetic
  source 1..161: mol_type = other DNA
aacgttcaacgactaatgagtgagttatacctcaacacgatacccccaaaatccactctatgagtgggtttaagatatagtctaaatatacccttaagggtgtaaagtataagttaaatattatacgtaatatattgtgcatttaggaggttttagtgtaa

SEQ ID NO: 46 - DNA, 145 bp, synthetic construct
  misc_feature 1..145: note = Synthetic
  source 1..145: mol_type = other DNA
aatttgaattggacaagatattgttgagtgctccttaaaataggtagggttattattatattttagaggcaattggggaaaattacaaggacatttaataaattacctccgttttattagattatttgtagcgaaacttaattcg

SEQ ID NO: 47 - DNA, 100 bp, synthetic construct
  misc_feature 1..100: note = Synthetic
  source 1..100: mol_type = other DNA
cgacatgcattccaagcttatgagttaacgtaagtcaagctcatgtaaaatctgcctaaaacgggaaactctcactgagacaatccgttgctaaatcagc

SEQ ID NO: 48 - DNA, 187 bp, synthetic construct
  misc_feature 1..187: note = Synthetic
  source 1..187: mol_type = other DNA
taaatgcccaacgactatccctgatgaatgtaagggagtagggtcaagcgacccgaaacggcagacaactctaagagttgaagatatagtctgaactgcatggtgacatgcagctgtttatataaacgggtagagaagtagcgaactctactgaacacattgatgaagtgaacacgttattcagttc

SEQ ID NO: 49 - DNA, 306 bp, synthetic construct
  misc_feature 1..306: note = Synthetic
  source 1..306: mol_type = other DNA
aacgttcaacgactaatgagtgagttatacctcaacacgatacccccaaaatccactctatgagtgggtttaagatatagtctaaatatacccttaagggtgtaaagtataagttaaatattatacgtaatatattgtgcatttaggaggttttagtgtaaaatttgaattggacaagatattgttgagtgctccttaaaataggtagggttattattatattttagaggcaattggggaaaattacaaggacatttaataaattacctccgttttattagattatttgtagcgaaacttaattcg

SEQ ID NO: 50 - DNA, 287 bp, synthetic construct
  misc_feature 1..287: note = Synthetic
  source 1..287: mol_type = other DNA
cgacatgcattccaagcttatgagttaacgtaagtcaagctcatgtaaaatctgcctaaaacgggaaactctcactgagacaatccgttgctaaatcagctaaatgcccaacgactatccctgatgaatgtaagggagtagggtcaagcgacccgaaacggcagacaactctaagagttgaagatatagtctgaactgcatggtgacatgcagctgtttatataaacgggtagagaagtagcgaactctactgaacacattgatgaagtgaacacgttattcagttc

SEQ ID NO: 51 - DNA, 741 bp, synthetic construct
  misc_feature 1..741: note = Synthetic
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 52 - DNA, 462 bp, synthetic construct
  misc_feature 1..462: note = Synthetic
  source 1..462: mol_type = other DNA
aagcaggtttccccaactgacacaaaccgtgcaacttgaaactccgcctggtctttccaggtctagaggggtgacactttgtactgtgcttgactccacgctcggtccactagcgggtgttagtaacagcactgttgtttcgtagcggagcatgatggccgtgggaactccgccttggtaacaaggacccacggggccgaaagccacgtcctaacggacccatcatgtgtgcaaccccagcacaacaacttttctgtgaaacctaccttaaggtgacgctgatactggtactcagacactggtgacaggctaaggatgcccttcaggtaccccgaggtaacacgcgacactcgggatctgagaaggggactagggcttctgtaaaagtgcctagtttaaaaagcttctatgcctgaataggtgaccggaggccggcacctttccctttacaactactgacta

SEQ ID NO: 53 - DNA, 750 bp, synthetic construct
  misc_feature 1..750: note = Synthetic
  source 1..750: mol_type = other DNA
ttaaaacagcctgtgggttgatcccacccacagggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagattgttaccatatagctattggattggccatccggtgaccaatagagctattatatatctctttgttgggtttataccacttagcttgaaagaggttaaaacattacaattcattgttaagttgaatacagcaaaatgggagc

SEQ ID NO: 54 - DNA, 696 bp, synthetic construct
  misc_feature 1..696: note = Synthetic
  source 1..696: mol_type = other DNA
ttgccagtctgctcgatatcgcaggctgggtccgtgactacccactccccctttcaacgtgaaggctacgatagtgccagggcgggtactgccgtaagtgccaccccaaacaacaacaacaaaacaaactccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat